(19) United States
(12) Patent Application Publication
(10) Pub. No.: US 2026/0305941 A1
(43) Pub. Date: Oct. 8, 2026

(54) METHODS FOR TISSUE IMAGING AND USES THEREOF

(71) Applicant: The University of Melbourne, Victoria (AU)

(72) Inventors: Laura Elizabeth Downie, Victoria (AU); Holly Rose Chinnery, Victoria (AU); Scott Norman Mueller, Victoria (AU); Mengliang Wu, Victoria (AU); Phillip Bedggood, Victoria (AU); Andrew Bernard Metha, Victoria (AU)

(73) Assignee: The University of Melbourne, Victoria (AU)

(21) Appl. No.: 19/476,554

(22) PCT Filed: Apr. 18, 2024
(86) PCT No.: PCT/AU2024/050373

§ 371 Date: Oct. 17, 2025

(30) Foreign Application Priority Data
Apr. 18, 2023 (AU) 2023901150

(51) Int. Cl. A61B 3/14; A61B 3/10; A61B 5/00

(52) U.S. Cl. CPC A61B 3/145; A61B 3/1005; A61B 3/1025; A61B 5/411; A61B 5/7275

(57) ABSTRACT

The present disclosure relates to methods for imaging biological tissue over time, and uses of the imaging methods. It relates particularly but not exclusively to imaging of live biological tissue, such as corneal or retinal tissue of a subject and allows for the dynamic (temporal) tracking of immune cells in the biological tissue.

### TECHNICAL FIELD

The present disclosure relates to methods for imaging biological tissue over time, and uses of the imaging methods. It relates particularly but not exclusively to imaging of live biological tissue, such as corneal or retinal tissue of a subject.

### BACKGROUND OF THE DISCLOSURE

The cornea is an avascular, highly innervated sensory tissue. The transparency and accessibility of the cornea affords detailed imaging and direct visualisation of its immune cells and other non-immune tissue cells.

The most commonly used in vivo confocal microscopy (IVCM) systems today are Confoscan Series (Nidek Co. Ltd, Gamagori, Japan) and the Heidelberg Retina Tomograph 3 (HRT-3) with Rostock Cornea Module (Heidelberg Engineering GmbH, Heidelberg Germany). Laser-scanning IVCM with the HRT-3 system has a lateral resolution of ~1 μm and axial resolution of ~4 μm, and delivers around 800-fold tissue magnification¹. Unlike conventional microscopes that involve diffuse illumination of a tissue sample, with IVCM, light is focused to a specific tissue depth, improving image resolution and contrast. Since the first description of confocal imaging to study the human eye ~30 years ago², IVCM has evolved as one of the major tools for examining the corneal micro-structure in clinical and research settings.

Static IVCM image analysis is the current mainstay for studying human corneal immune cells³. This approach involves analysing cell density and morphology and has been largely limited to the epithelium. Most published reports have interpreted IVCM findings with a view that only dendritic cells (DCs) exist in the human corneal epithelium, as described in studies on mice in the steady state⁴. Based on this assumption, two main morphological subtypes of “presumed DCs” have been defined: “mature DCs” (>25 μm with long dendrites) and “immature DCs” (≤25 μm without obvious dendrites)⁵. However, key limitations of static IVCM are the inability to analyse cell behaviours, or to reliably disambiguate immune cells in the corneal stroma from the cell nuclei of fixed stromal keratocytes.

It would therefore be desirable to provide a method for imaging biological tissue over time using microscopy that enables analysis of cell behaviour and/or differentiation of cell types and/or cell interactions.

### SUMMARY OF THE DISCLOSURE

The present inventors have developed a new IVCM technique that enables dynamic (temporal) tracking of immune cells in a biological tissue. The present inventors used this method to track immune cells over the corneal tissue depth in living human eyes. High-resolution volume and/or sequence scans of the cornea were acquired and en face images were registered to fixed anatomical features to generate time-lapsed videos. Advantageously, unlike static IVCM, this new IVCM technique provides dimensionality to human in vivo immune cell analysis, with label-free longitudinal evaluation of cell density, morphology and dynamics through the tissue depth. Images can advantageously be acquired at different tissue eccentricities to assess for regional effects.

The present inventors have demonstrated the use of this new IVCM technique to define the dynamic behaviours of distinct morphological immune cell subsets in the healthy human cornea. They have provided the first in vivo descriptions of putative corneal epithelial dendritic cell (DC) and T cell surveillance behaviours, stromal immune cell dynamics, as well as immune cell-to-cell and neuro-immune interactions in a physiologically intact human tissue. Moreover, the inventors have shown this new IVCM technique to be a unique and powerful tool for evaluating behavioural changes in corneal immune cells under non-homeostatic conditions, modelled, for example, by chronic immune activation due to seasonal allergy⁶ and an acute pro-inflammatory stimulus induced by contact lens (CL) wear⁷.

In one aspect, the present disclosure provides a method for imaging a biological tissue over time (e.g., including imaging of a biological tissue of a living subject), the method comprising the steps of: providing microscopy images of the biological tissue acquired over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identifying at least one microscopy image acquired at the first time point and the second time point; and producing a sequence of the identified microscopy images, thereby imaging the biological tissue over the time period.

In some embodiments, the at least one microscopy image is identified based on a desired focal depth (z) for imaging of the biological tissue. The desired focal depth (z) may be a depth of the biological tissue within a tissue layer. For example, where the biological tissue is corneal tissue, the desired focal depth (z) may be a depth in the epithelium or the stroma. In some embodiments, the at least one microscopy image is identified based on the same focal depth (z) at the first time point and the second time point. The focal depth (z) may be in a range of about 50 μm to about 100 μm (for example, about 60 μm to about 80 μm, or about 80 μm to about 100 μm) for imaging of the stroma of corneal tissue. A skilled person would recognise that different corneas may have different epithelial thicknesses. In some embodiments, the focal depth (z) (from the most apical aspect of the corneal epithelium), may be positioned below the anterior limiting lamina (Bowman's membrane) at between about 50 to 100 μm but typically in the range of 60 to 80 μm when imaging the stroma.

In some embodiments, a plurality of microscopy images is acquired at the first time point and the second time point, and wherein the plurality of microscopy images is acquired at the first location within the focal plane (x, y) of the biological tissue and across a predetermined range of focal depths (z) of the biological tissue. The plurality of microscopy images acquired at the first time point and the second time point may provide for volume imaging across a depth of the biological tissue while at a fixed location in the focal plane (x, y). The fixed location may refer to the microscopy images being acquired at or about the same position in the focal plane (x, y) although at various depths within the z-plane. The predetermined range of focal depths (z) may be in a range of about 50 μm to about 100 μm (for example, about 60 μm to about 80 μm, or about 80 μm to about 100 μm). The plurality of microscopy images acquired may comprise at least two images. The plurality of microscopy images may comprise about 2 images to about 40 images. Preferably, each microscopy image is acquired at a different focal depth (z) to produce a volume scan at the first location.

Additionally/alternatively, a plurality of microscopy images may be acquired at the first time point and the second time point, and wherein the plurality of microscopy images is a series of repeated images acquired at the first location within the focal plane (x, y) at the focal depth (z) of the biological tissue. The plurality of microscopy images acquired at the first time point and the second time point may provide for sequential imaging of the biological tissue at the first location through the repeated images acquired at or about the same position in the focal plane (x, y). The repeated images may be acquired at the first location at or about the same position within the focal plane (x, y). Alternatively, the repeated images may be acquired at the first location at different positions within the focal plane (x, y). The series of repeated images may comprise at least two images. The series of repeated images may comprise about 2 images to about 100 images. Preferably, each microscopy image is acquired at a different position in the focal plane (x, y) to produce a sequential scan at or about the first location.

In some embodiments, providing the microscopy images further comprises providing one or both of: one or more additional microscopy images acquired at the first time point over the time period, wherein the one or more additional microscopy images are acquired at a second location within the focal plane (x, y) of the biological tissue and at the focal depth (z) of the biological tissue; and one or more additional microscopy images acquired at the second time point over the time period, wherein the one or more additional microscopy images are acquired at the second location within the focal plane (x, y) of the biological tissue and at the same or a different focal depth (z) of the biological tissue. In some embodiments, a plurality of microscopy images may be acquired at the first time point and the second time point at the second location.

In some embodiments, identifying the at least one microscopy image comprises: identifying at least one microscopy image acquired at the first time point and the second time point for each of the first location and the second location; and producing a first sequence of the identified microscopy images at the first location and a second sequence of the identified microscopy images at the second location. Thus, two sequences of microscopy images may be produced, one for each location.

The sequence of identified microscopy images, which may comprise a first sequence and/or a second sequence, may consist of an ordered sequence of the identified microscopy images. The ordering may be based on time of acquisition of the microscopy images. The ordering may or may not be sequential. Additionally/alternatively, the ordering may be based on at least one feature of the biological tissue in the microscopy images e.g., an object or cell to be tracked through the images, or optionally, one or more anatomical features of the biological tissue.

In some embodiments, providing the microscopy images further comprises providing: one or more microscopy images acquired at one or more subsequent time points over the time period, wherein the one or more microscopy images are acquired at the first location within the focal plane (x, y) of the biological tissue and at the same or a different focal depth (z) of the biological tissue to the first and second time points, and wherein identifying the least one microscopy image further comprises identifying at least one microscopy image acquired at the one or more subsequent time points. The one or more subsequent time points may be a third time point, a fourth time point, a fifth time point, etc.

The method may further comprise determining the presence of at least one object in one or more of the identified microscopy images of the biological tissue over the time period.

The at least one object may be static over the time period, that is, the object may not move or be motile. The presence of the at least one object which is static may be determined based on one or more characteristics selected from at least one of a group comprising: location within the focal plane (x, y) of the biological tissue, focal depth (z) of the biological tissue, reflectivity, and one or more morphological parameters. The one or more morphological parameters may be selected from at least one of a group comprising: object field area, object area, object perimeter, object circularity, object roundness, object solidity, and dendritic complexity. The at least one object which is static may be a non-immune cell. The non-immune cell may be a fibroblast. In particular, the non-immune cell may be a corneal keratocyte where the biological tissue is corneal tissue.

The at least one object may be dynamic over the time period, that is, the object may be moving or motile. The presence of the at least one object which is dynamic may be determined based on one or more characteristics selected from at least one of a group comprising: location within the focal plane (x, y) of the biological tissue, focal depth (z) of the biological tissue, reflectivity, one or more morphological parameters, and one or more dynamic parameters. The one or more morphological parameters may be selected from at least one of a group comprising: object field area, object area, object perimeter, object circularity, object roundness, object solidity, and dendritic complexity. The one or more morphological parameters may comprise object field area, object area and object solidity, particularly where the dynamic object is a cell.

In some embodiments, the method further comprises monitoring dynamic behaviour of the at least one object in one or more of the identified microscopy images of the biological tissue over the time period. The step of monitoring the dynamic behaviour of the at least one object may comprise monitoring one or more dynamic parameters of the at least one object over the time period.

The one or more dynamic parameters may be selected from at least one of a group comprising: instantaneous speed, lower arrest coefficient, object displacement speed, meandering index, displacement ratio, linearity of forward progression, mean directional change, dendrite surveillance extension and retraction cycling habitude (dSEARCH), probing speed, outreach ratio, and dancing index.

In some embodiments, the method further comprises the step of tracking movement of the at least one object across a range of focal depths (z) of the biological tissue over the time period.

In some embodiments, the at least one object is a cell, and the method further comprises the step of: determining the cell type based on the monitored dynamic behaviour of the at least one cell in the one or more identified microscopy images of the biological tissue over the time period. The at least one cell may be an immune cell. The cell type may be selected from one of a group comprising: a putative T cell, a putative dendritic cell, and a putative macrophage.

In some embodiments, the method further comprises determining the presence of at least two objects in one or more of the identified microscopy images of the biological tissue over the time period; and monitoring dynamic behaviour of the at least two objects in one or more of the identified microscopy images of the biological tissue over the time period. The method may further comprise the step of tracking movement of the at least two objects across a range of focal depths (z) of the biological tissue over the time period.

In some embodiments, the method further comprises the step of: assessing an object-to-object interaction of the at least two objects in one or more of the identified microscopy images over the time period. The step of assessing the object-to-object interaction may comprise one or both of the following steps: determining a change in dynamic behaviour of one or more of the at least two objects in one or more of the identified microscopy images before and after the object-to-object interaction; and determining the number of times that the at least two objects interact over the time period.

The at least two objects may be cells. The at least two cells may be immune cells. The at least two cells may be different cell types. The at least two cells may be the same cell type. The cell type may be selected from one of a group comprising: a putative T cell, a putative dendritic cell, or a putative macrophage.

In some embodiments, the method further comprises the steps of determining the presence of at least one nerve in one or more of the identified microscopy images, and monitoring dynamic behaviour of the at least one nerve in one or more of the identified microscopy images over the time period.

The method may further comprise the step of assessing an object-to-nerve interaction of the at least one object and the at least one nerve in one or more of the identified microscopy images over the time period. Assessing the object-to-nerve interaction may comprise one or both of the following steps: determining a change in dynamic behaviour of the least one object and the at least one nerve before and after the object-to-nerve interaction; and determining the number of times that the at least one object and the at least one nerve interact over the time period.

In some embodiments, the method further comprises the steps of: providing microscopy images of the biological tissue over the time period acquired in a subsequent acquisition at a specified interval from an initial acquisition of the microscopy images; identifying at least one microscopy image from the subsequent acquisition acquired at the first time point and the second time point; and producing a sequence of the identified microscopy images from the subsequent acquisition, thereby imaging the biological tissue over the time period. The specified interval between the initial acquisition and the subsequent acquisition may be in a range of about 30 minutes to about 24 hours, and more preferably in a range of about 15 minutes to about 8 hours.

In some embodiments, the method further comprises the steps of determining the presence of at least one object in one or more of the identified microscopy images in the subsequent acquisition based on one or more characteristics; and monitoring dynamic behaviour of the at least one object in one or more of the identified microscopy images in the subsequent acquisition over the time period.

The method may further comprise the step of comparing the monitored dynamic behaviour of the at least one object in the subsequent acquisition from the initial acquisition.

The method may further comprise the step of determining a pattern of change in the monitored dynamic behaviour for the at least one object. The pattern of change may be associated with a change in immune status of the biological tissue.

In some embodiments, the method further comprises the step of identifying a condition or disease state based on the change in immune status of the biological tissue. The condition or disease state may be selected from at least one of a group comprising: acute or chronic inflammation of the tissue; an ocular disease or condition; systemic disease or condition; and allergic conditions.

Ocular diseases or conditions include those that affect eye health, for example, but not limited to, corneal grafts, post-refractive surgery, lagophthalmos, wound healing, corneal infections (e.g., herpetic keratitis), uveitis, sterile inflammation, dry eye disease, contact lens wear, keratoconus, corneal dystrophies and degenerations, graft versus host disease (GVHD), limbal stem cell deficiency, corneal erosions, stem cell deficiency, epitheliopathy, injury, trauma, fibrosis or conditions that primarily affect the posterior eye (e.g., glaucoma, retinal disease, etc.).

Systemic diseases or conditions include those that affect the immune system, for example, but not limited to, infections (e.g., Human Immunodeficiency Virus (HIV), COVID-19), auto-immune diseases (e.g., Systemic Lupus Erythematosus (SLE), Rheumatoid Arthritis (RA), Sjogren's disease), genetic forms of immune dysfunction, menopause, hormonal changes, etc.; and systemic diseases or conditions that affect the nervous system (for example, but not limited to, diabetes, post-stroke, neurodegenerative diseases (e.g., Multiple Sclerosis, dementia, Alzheimer's disease, Parkinson's disease, small fibre neuropathy, etc.), cancers. In some embodiments, the method identifies changes in corneal immune cell behaviours associated with HIV infection.

In some embodiments, the method further comprises the step of identifying a change in immune status of the biological tissue, unrelated to a condition or disease state. For example, the method may be used to identify a change in immune status associated with environmental factors (e.g., seasonal-driven changes in one or more of, for example, temperature, humidity, rainfall, particulate matter concentration, and grass pollen concentration), or temporal (diurnal) factors.

In some embodiments, the method further comprises the steps of: providing an agent to the biological tissue (e.g., topical administration to the cornea) or to a subject having the biological tissue (e.g., by inhalation, or intravenous administration) before the subsequent acquisition; and assessing an effect of the agent on the biological tissue after the subsequent acquisition based on the comparison of the dynamic behaviour of the at least one object in the subsequent acquisition from the initial acquisition.

The agent may be selected from at least one of a group comprising: an environmental agent; an exogenous agent; a neurostimulatory agent; a dietary supplement (e.g., fish oil), and a therapeutic agent.

The therapeutic agent may be a vaccine, an eye drop, or an immunomodulator (e.g., an anti-inflammatory drug). The agent may be a nerve stimulatory agent, such as a menthol-containing product, or capsicum spray. The environmental agent may include light, smoke or pollutant exposure. The exogenous agent may include eye rubbing, caffeine intake, or exercise. The agent may be a hyperosmotic or a hypoosmotic stimulus.

In some embodiments, the method further comprises the steps of applying a medical device or medical therapy to the biological tissue or to a subject having the biological tissue before the subsequent acquisition; and assessing an effect of the medical device or medical therapy on the biological tissue after the subsequent acquisition based on the comparison of the dynamic behaviour of the at least one object in the subsequent acquisition from the initial acquisition. The medical device may be a contact lens.

The at least one object may be an intrinsic or extrinsic feature of the biological tissue. The intrinsic feature may comprise one or more of a nerve or part thereof (e.g., an axon), a pattern of nerves, a cell or part thereof, a pattern of cells and at least one feature of connective tissue. The extrinsic feature may comprise one or more of a cell, a pattern of cells, and an organism (either living or non-living), or a foreign-body entity, to name a few. The organism may comprise one of an amoeba, a bacteria or a fungi. In particular, the organism may comprise Acanthamoeba cysts, bacteria or fungal filaments, which are found e.g., in corneal infection.

In some embodiments, the at least one microscopy image is identified based on one or more features of the biological tissue. The one or more features of the biological tissue may comprise one or more of a nerve, a pattern of nerves, a cell, a pattern of cells, and at least one feature of connective tissue. The at least one microscopy image may be identified based on the presence of at least one cell.

In some embodiments, the method further comprises the steps of: analysing the identified microscopy images to locate one or more anatomical landmarks; and registering the identified microscopy images to the one or more anatomical landmarks. The one or more anatomical landmarks may comprise one or more of a nerve, a pattern of nerves, a cell, a pattern of cells, and at least one feature of connective tissue. The method may further comprise assigning a location identifier to the identified microscopy images based on the one or more anatomical landmarks. The location identifier may be assigned based on the same anatomical landmark. The location identifier may be e.g., a numerical or alphabetical code assigned to the image, indicative of an anatomical landmark. The location identifier may be used in the method for ordering of the images into a sequence and/or display of the images, and/or determining the presence of at least one object of the biological tissue such as for tracking of the object (e.g., a cell).

In some embodiments, the method further comprises the step of producing a time-lapsed video from the sequence of the identified microscopy images at a desired frame rate. The time-lapsed video may be in colour. The desired frame rate may be in range of about 1 to about 10 frames/second, preferably about 1 to about 5 frames/second, and more preferably about 3 frames/second.

In some embodiments, the method further comprises the step of producing a combined image from the sequence of the identified microscopy images by overlaying the images to form a two-dimensional (2D) representation. The combined image may comprise one or more colours representing changes to one or more features of the biological tissue over the time period.

In some embodiments, the method further comprises the step of: producing a first time-lapsed video from the first sequence of the identified microscopy images at the first location and a second time-lapsed video from the second sequence of the identified microscopy images at the second location. The method may further comprise the step of: producing a combined image for each of the first sequence of the identified microscopy images and the second sequence of the identified microscopy images by overlaying the respective images to form a two-dimensional (2D) representation. Each of the combined images may comprise one or more colours representing changes to one or more features of the biological tissue over the time period.

In some embodiments, the microscopy images are acquired at a plurality of time points at a predetermined frequency over the time period. The predetermined frequency may be in a range of about 2 minutes to about 9 minutes and in particular about 4 to about 5 minutes. Each time point may comprise a time range of more than 0 minutes to about 2 minutes. The time period may be no more than about 60 minutes. The time period may be between about 20 minutes and about 60 minutes.

The step of providing the microscopy images may comprise acquiring the microscopy images of the biological tissue over the time period. The step of acquiring the microscopy images may comprise the step of operating an imaging system configured to acquire en face microscopy images of the biological tissue over the time period. The imaging system may comprise one of a confocal microscopy system or an optical coherence microscopy (OCM) system. The confocal microscopy imaging system may be in vivo confocal microscopy (IVCM) system. The method may comprise providing in vivo microscopy images of the biological tissue over the time period.

In some embodiments, the biological tissue is a living tissue. The biological tissue may be a connective tissue. The connective tissue may comprise an epithelium and a stroma.

The microscopy images acquired at the first time point and/or the second time point may be at a focal depth (z) within the epithelium of the biological tissue. In other embodiments, the microscopy images are acquired at the first time point and/or the second time point are at a focal depth (z) within the stroma of the biological tissue.

In further embodiments, the microscopy images acquired at the first time point are at a focal depth (z) within the epithelium or stroma of the biological tissue, and the microscopy images acquired at the second time point are at a focal depth (z) within the other of the epithelium or stroma of the biological tissue.

In further embodiments, the microscopy images acquired at the one or more subsequent time points are at a focal depth (z) within the epithelium or the stroma of the biological tissue.

The biological tissue may be tissue from an eye of a subject. The biological tissue may be ocular surface tissue. The biological tissue may be corneal tissue.

The first location in the focal plane (x, y) of the biological tissue may be one of the following: a corneal tissue region of the subject's eye comprising one of a central region, a paracentral region, a peripheral region, or an inferior whorl region, a conjunctival region of the subject's eye, or a limbal region of the subject's eye.

The second location in the focal plane (x, y) of the biological tissue may be one of the following: a corneal tissue region of the subject's eye comprising one of a central region, a paracentral region, a peripheral region, or an inferior whorl region, a conjunctival region of the subject's eye, or a limbal region of the subject's eye.

The first location may be the inferior whorl region and the second location may be the peripheral region.

In another aspect, the present disclosure provides a method for imaging a biological tissue over time, the method comprising the steps of: providing microscopy images of the biological tissue acquired over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue, identifying at least one microscopy image acquired at the first time point and the second time point; and producing a sequence of the identified microscopy images, thereby imaging the biological tissue over the time period.

The repeated images may be acquired at the first location and the second location at or about the same position within the focal plane (x, y). In other embodiments, the repeated images are acquired at the first location and the second location at different positions within the focal plane (x, y). The series of repeated images comprises about 2 images to about 100 images.

In some embodiments, the method comprises identifying at least one microscopy image acquired at the first time point and the second time point for each of the first location and the second location, and producing a first sequence of the identified microscopy images at the first location and a second sequence of the identified microscopy images at the second location.

The sequence of identified microscopy images, which may comprise a first sequence and/or a second sequence, may consist of an ordered sequence of the identified microscopy images. The ordering may be based on time of acquisition of the microscopy images. The ordering may or may not be sequential. Additionally/alternatively, the ordering may be based on at least one feature of the biological tissue in the microscopy images e.g., an object or cell to be tracked through the images, or optionally, one or more anatomical features of the biological tissue.

The at least one microscopy image may be identified based on a desired position within the focal plane (x, y) for imaging of the biological tissue. The at least one microscopy image may be identified based on the same or substantially the same position within the focal plane (x, y) at the first time point and the second time point.

The method may further comprise determining the presence of at least one object in one or more of the identified microscopy images of the biological tissue over the time period.

The at least one object may be static over the time period, that is, the object may not move or be motile. The presence of the at least one object which is static may be determined based on one or more characteristics selected from at least one of a group comprising: location within the focal plane (x, y) of the biological tissue, focal depth (z) of the biological tissue, reflectivity, and one or more morphological parameters. The one or more morphological parameters may be selected from at least one of a group comprising: object field area, object area, object perimeter, object circularity, object roundness, object solidity, and dendritic complexity. The at least one object which is static may be a non-immune cell. The non-immune cell may be a fibroblast. In particular, the non-immune cell may be a corneal keratocyte where the biological tissue is corneal tissue.

The at least one object may be dynamic over the time period, that is, the object may be moving or motile. The presence of the at least one object which is dynamic may be determined based on one or more characteristics selected from at least one of a group comprising: location within the focal plane (x, y) of the biological tissue, focal depth (z) of the biological tissue, reflectivity, one or more morphological parameters, and one or more dynamic parameters. The one or more morphological parameters may be selected from at least one of a group comprising: object field area, object area, object perimeter, object circularity, object roundness, object solidity, and dendritic complexity. The one or more morphological parameters may comprise object field area, object area and object solidity, particularly where the dynamic object is a cell.

In some embodiments, the method further comprises monitoring dynamic behaviour of the at least one object in one or more of the identified microscopy images of the biological tissue over the time period. The step of monitoring the dynamic behaviour of the at least one object may comprise monitoring one or more dynamic parameters of the at least one object over the time period.

The one or more dynamic parameters may be selected from at least one of a group comprising: instantaneous speed, lower arrest coefficient, object displacement speed, meandering index, displacement ratio, linearity of forward progression, mean directional change, dendrite surveillance extension and retraction cycling habitude (dSEARCH), probing speed, outreach ratio, and dancing index.

In some embodiments, the method further comprises the step of tracking movement of the at least one object across a range of focal depths (z) of the biological tissue over the time period.

In some embodiments, the at least one object is a cell, and the method further comprises the step of: determining the cell type based on the monitored dynamic behaviour of the at least one cell in the one or more identified microscopy images of the biological tissue over the time period. The at least one cell may be an immune cell. The cell type may be selected from one of a group comprising: a putative T cell, a putative dendritic cell, or a putative macrophage.

In some embodiments, the method further comprises determining the presence of at least two objects in one or more of the identified microscopy images of the biological tissue over the time period; and monitoring dynamic behaviour of the at least two objects in one or more of the identified microscopy images of the biological tissue over the time period. The method may further comprise the step of tracking movement of the at least two objects across a range of focal depths (z) of the biological tissue over the time period.

In some embodiments, the method further comprises the step of: assessing an object-to-object interaction of the at least two objects in one or more of the identified microscopy images over the time period. The step of assessing the object-to-object interaction may comprise one or both of the following steps: determining a change in dynamic behaviour of one or more of the at least two objects in one or more of the identified microscopy images before and after the object-to-object interaction; and determining the number of times that the at least two objects interact over the time period.

The at least two objects may be cells. The at least two cells may be immune cells. The at least two cells may be different cell types. The at least two cells may be the same cell type. The cell type may be selected from one of a group comprising: a putative T cell, a putative dendritic cell, and a putative macrophage.

In some embodiments, the method further comprises the steps of determining the presence of at least one nerve in one or more of the identified microscopy images, and monitoring dynamic behaviour of the at least one nerve in one or more of the identified microscopy images over the time period.

The method may further comprise the step of assessing an object-to-nerve interaction of the at least one object and the at least one nerve in one or more of the identified microscopy images over the time period. Assessing the object-to-nerve interaction may comprise one or both of the following steps: determining a change in dynamic behaviour of the least one object and the at least one nerve before and after the object-to-nerve interaction; and determining the number of times that the at least one object and the at least one nerve interact over the time period.

In some embodiments, the method further comprises the steps of: providing microscopy images of the biological tissue over the time period acquired in a subsequent acquisition at a specified interval from an initial acquisition of the microscopy images; identifying at least one microscopy image from the subsequent acquisition acquired at the first time point and the second time point; and producing a sequence of the identified microscopy images from the subsequent acquisition, thereby imaging the biological tissue over the time period. The specified interval between the initial acquisition and the subsequent acquisition may be in a range of about 30 minutes to about 24 hours, and more preferably in a range of about 15 minutes to about 8 hours.

In some embodiments, the method further comprises the steps of determining the presence of at least one object in one or more of the identified microscopy images in the subsequent acquisition based on one or more characteristics; and monitoring dynamic behaviour of the at least one object in one or more of the identified microscopy images in the subsequent acquisition over the time period.

The method may further comprise the step of comparing the monitored dynamic behaviour of the at least one object in the subsequent acquisition from the initial acquisition.

The method may further comprise the step of determining a pattern of change in the monitored dynamic behaviour for the at least one object. The pattern of change may be associated with a change in immune status of the biological tissue.

In some embodiments, the method further comprises the step of identifying a condition or disease state based on the change in immune status of the biological tissue. The condition or disease state may be selected from at least one of a group comprising: acute or chronic inflammation of the tissue; an ocular disease or condition; a systemic disease or condition; and allergic conditions.

In some embodiments, the method further comprises the steps of: providing an agent to the biological tissue or to a subject having the biological tissue before the subsequent acquisition; and assessing an effect of the agent on the biological tissue after the subsequent acquisition based on the comparison of the dynamic behaviour of the at least one object in the subsequent acquisition from the initial acquisition. The agent may be selected from at least one of a group comprising: an environmental agent; an exogenous agent; a neurostimulatory agent; a dietary supplement (e.g., fish oil), and a therapeutic agent. The therapeutic agent may be an immunomodulator.

In some embodiments, the method further comprises the steps of applying a medical device or medical therapy to the biological tissue or to a subject having the biological tissue before the subsequent acquisition; and assessing the effect of the medical device or medical therapy on the biological tissue after the subsequent acquisition based on the comparison of the dynamic behaviour of the at least one object in the subsequent acquisition from the initial acquisition. The medical device may be a contact lens.

The at least one object may be an intrinsic or extrinsic feature of the biological tissue. The intrinsic feature may comprise one or more of a nerve or part thereof (e.g., an axon), a pattern of nerves, a cell or part thereof, a pattern of cells and at least one feature of connective tissue of the biological tissue. The extrinsic feature may comprise one or more of a cell, a pattern of cells, and an organism (either living or non-living), or a foreign-body entity, to name a few. The organism may comprise one of an amoeba, a bacteria or a fungi. In particular, the organism may comprise Acanthamoeba cysts, bacteria or fungal filaments, which are found e.g., in corneal infection.

In some embodiments, the at least one microscopy image is identified based on one or more features of the biological tissue. The one or more features of the biological tissue may comprise one or more of a nerve, a pattern of nerves, a cell, a pattern of cells, and at least one feature of connective tissue. The at least one microscopy image may be identified based on the presence of at least one cell.

In some embodiments, the method further comprises the steps of: analysing the identified microscopy images to locate one or more anatomical landmarks; and registering the identified microscopy images to the one or more anatomical landmarks. The one or more anatomical landmarks may comprise one or more of a nerve, a pattern of nerves, a cell, a pattern of cells, and at least one feature of connective tissue. The method may further comprise assigning a location identifier to the identified microscopy images based on the one or more anatomical landmarks. The location identifier may be assigned based on the same anatomical landmark. The location identifier may be e.g., a numerical or alphabetical code assigned to the image, indicative of an anatomical landmark. The location identifier may be used in the method for ordering of the images into a sequence and/or display of the images, and/or determining the presence of at least one object of the biological tissue such as for tracking of the object (e.g., a cell).

In some embodiments, the method further comprises the step of producing a time-lapsed video from the sequence of the identified microscopy images at a desired frame rate. The time-lapsed video may be in colour. The desired frame rate may be in range of about 1 to about 10 frames/second, preferably about 1 to about 5 frames/second, and more preferably about 3 frames/second.

In some embodiments, the method further comprises the step of producing a combined image from the sequence of the identified microscopy images by overlaying the images to form a two-dimensional (2D) representation. The combined image may comprise one or more colours representing changes to one or more features of the biological tissue over the time period.

In some embodiments, the method further comprises the step of: producing a first time-lapsed video from the first sequence of the identified microscopy images at the first location and a second time-lapsed video from the second sequence of the identified microscopy images at the second location. The method may further comprise the step of: producing a combined image for each of the first sequence of the identified microscopy images and the second sequence of the identified microscopy images by overlaying the respective images to form a two-dimensional (2D) representation. Each of the combined images may comprise one or more colours representing changes to one or more features of the biological tissue over the time period.

In some embodiments, the microscopy images are acquired at a plurality of time points at a predetermined frequency over the time period. The predetermined frequency may be in a range of about 2 minutes to about 9 minutes and in particular about 4 to about 5 minutes. Each time point may comprise a time range of more than 0 minutes to about 2 minutes. The time period may be no more than about 60 minutes. The time period may be between about 20 minutes and about 60 minutes.

The step of providing the microscopy images may comprise acquiring the microscopy images of the biological tissue over the time period. The step of acquiring the microscopy images may comprise the step of operating an imaging system configured to acquire en face microscopy images of the biological tissue over the time period. The imaging system may comprise one of a confocal microscopy system and an optical coherence microscopy (OCM) system. The confocal microscopy imaging system may be in vivo confocal microscopy (IVCM) system. The method may comprise providing in vivo microscopy images of the biological tissue over the time period.

In some embodiments, the biological tissue is a living tissue. The biological tissue may be a connective tissue. The connective tissue may comprise an epithelium and a stroma.

The microscopy images acquired at the first time point and/or the second time point may be at a focal depth (z) within the epithelium of the biological tissue. In other embodiments, the microscopy images are acquired at the first time point and/or the second time point are at a focal depth (z) within the stroma of the biological tissue.

In further embodiments, the microscopy images acquired at the first time point are at a focal depth (z) within the epithelium or stroma of the biological tissue, and the microscopy images acquired at the second time point are at a focal depth (z) within the other of the epithelium or stroma of the biological tissue.

In further embodiments, the microscopy images acquired at the one or more subsequent time points are at a focal depth (z) within the epithelium or the stroma of the biological tissue.

The biological tissue may be tissue from an eye of a subject. The biological tissue may be ocular surface tissue. The biological tissue may be corneal tissue.

The first location in the focal plane (x, y) of the biological tissue may be one of the following: a corneal tissue region of the subject's eye comprising one of a central region, a paracentral region, a peripheral region, or an inferior whorl region, a conjunctival region of the subject's eye, or a limbal region of the subject's eye.

The second location in the focal plane (x, y) of the biological tissue is one of the following: a corneal tissue region of the subject's eye comprising one of a central region, a paracentral region, a peripheral region, or an inferior whorl region, a conjunctival region of the subject's eye, or a limbal region of the subject's eye.

The first location may be the inferior whorl region and the second location may be the peripheral region.

In another aspect, the present disclosure provides a method for imaging a biological tissue over time, the method comprising the steps of: acquiring microscopy images of the biological tissue over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identifying at least one microscopy image acquired at the first time point and the second time point; and producing a sequence of the identified microscopy images, thereby imaging the biological tissue over the time period.

In another aspect, the present disclosure provides a method for imaging a biological tissue over time, the method comprising the steps of: acquiring microscopy images of the biological tissue over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue; identifying at least one microscopy image acquired at the first time point and the second time point; and producing a sequence of the identified microscopy images, thereby imaging the biological tissue over the time period.

In another aspect, the present disclosure provides a computer-implemented method for imaging a biological tissue over time, the computer-implemented method comprising the following steps each being performed by a processor: receiving microscopy images of the biological tissue acquired over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identifying at least one microscopy image acquired at the first time point and the second time point; and producing a sequence of the identified microscopy images, thereby imaging the biological tissue over the time period.

In another aspect, the present disclosure provides a system for imaging a biological tissue over time, the system comprising: a communication interface configured to receive microscopy images of the biological tissue acquired over a time period; a processor; and a computer-readable medium communicatively coupled to the processor and storing computer-executable instructions that, when executed by the processor, cause the processor to: receive, via the communication interface: one or more microscopy images acquired at a first time point over the time period, wherein the one or more microscopy images are acquired at the first location within the focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identify at least one microscopy image acquired at the first time point and the second time point; and produce a sequence of the identified microscopy images, thereby imaging the biological tissue over the time period.

In another aspect, the present disclosure provides a computer-implemented method for imaging a biological tissue over time, the computer-implemented method comprising the following steps each being performed by a processor: receiving microscopy images of the biological tissue over a time period, comprising a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue; identifying at least one microscopy image acquired at the first time point and the second time point; and producing a sequence of the identified microscopy images, thereby imaging the biological tissue over the time period.

In another aspect, the present disclosure provides a system for imaging a biological tissue over time, the system comprising: a communication interface configured to receive microscopy images of the biological tissue acquired over a time period; a processor; and a computer-readable medium communicatively coupled to the processor and storing computer-executable instructions that, when executed by the processor, cause the processor to: receive, via the communication interface: a plurality of microscopy images acquired at a first time point over the time period, wherein the plurality of microscopy images is a series of repeated images acquired at a first location and at a second location within the focal plane (x, y) of the biological tissue; and a plurality of microscopy images acquired at a second time point over the time period, wherein the plurality of microscopy images are a series of repeated images acquired at the first location and at the second location within the focal plane (x, y) of the biological tissue; identify at least one microscopy image acquired at the first time point and the second time point; and produce a sequence of the identified microscopy images, thereby imaging the biological tissue over the time period.

In another aspect, the present disclosure provides a method for evaluating cellular changes or dynamic behaviour of at least one immune cell in corneal tissue of a subject over time, the method comprising the steps of: providing microscopy images of the corneal tissue acquired over a time period, comprising: one or more microscopy images acquired at a first time point over the time period, wherein the one or more microscopy images are acquired at a first location within a focal plane (x, y) of the corneal tissue and at a focal depth (z) of the corneal tissue; and one or more microscopy images acquired at a second time point over the time period, wherein the one or more microscopy images are acquired at the first location within the focal plane (x, y) of the corneal tissue and at the same or a different focal depth (z) of the corneal tissue; identifying at least one microscopy image acquired at the first time point and the second time point; producing a sequence of the identified microscopy images; determining the presence of at least one immune cell in one or more of the identified microscopy images based on one or more characteristics (e.g., the morphology, or number, or density, or dynamic behaviour of the at least one immune cell); and evaluating cellular changes or dynamic behaviour of the at least one immune cell in one or more of the identified microscopy images over the time period.

In another aspect, the present disclosure provides a method for evaluating cellular changes or dynamic behaviour of at least one immune cell in corneal tissue of a subject over time, the method comprising the steps of: providing microscopy images of the corneal tissue acquired over a time period, comprising: a plurality of microscopy images acquired at a first time point over the time period, wherein the plurality of microscopy images is a series of repeated images are acquired at a first location and at a second location within a focal plane (x, y) of the corneal tissue; and a plurality of microscopy images acquired at a second time point over the time period, wherein the plurality of microscopy images is a series of repeated images acquired at the first location and at the second location within the focal plane (x, y) of the corneal tissue; identifying at least one microscopy image acquired at the first time point and the second time point; producing a sequence of the identified microscopy images; determining the presence of at least one immune cell in one or more of the identified microscopy images based on one or more characteristics (e.g., the morphology, or number, or density, or dynamic behaviour of the at least one immune cell); and evaluating cellular changes or dynamic behaviour of the at least one immune cell in one or more of the identified microscopy images over the time period.

In another aspect, the present disclosure provides a method of identifying a condition or disease state in a subject, the method comprising the steps of: a) determining the morphology, or number, or density, or dynamic behaviour of one or more immune cells at one or more locations within a focal plane (x, y) and/or at one or more focal depths (z) in corneal tissue of the subject over time; and b) comparing the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject.

Advantageously, this method allows for increased accuracy with, for example, regard to cell density counts and/or the capacity to distinguish between moving objects such as immune cells and connective tissue cells, particularly in the stroma (where, for example, macrophages may not be distinguished from keratocytes), and/or the capacity to distinguish between immune cells, for example in the epithelium (where T cells move around and sometimes can be seen to “kiss” (i.e., physical interaction, typically a transient interaction, between 2 objects) a dendritic cell or a nerve in a static image preventing its accurate identification as a T cell or where globular (stationary) cells in the whorl region may not be distinguished from dynamic T cells). In some embodiments, the reference level is the morphology, number, or average density, or dynamic behaviour of the one or more immune cells at the one or more locations within the focal plane (x, y) and/or at the one or more focal depths (z) in the corneal tissue of a healthy subject or a subject treated with or exposed to an agent or a baseline level of the subject (e.g., prior to treatment with or exposure to an environmental agent, an exogenous agent, a neurostimulatory agent, or a therapeutic agent).

In some embodiments, one or more immune cells are “matched”, meaning that the morphology, or number, or density, or dynamic behaviour of the same cell(s) is determined over time, for example, at a first and second time point.

In other embodiments, a group of cells belonging to the same subset (e.g., all putative dendritic cells, or all putative T cells or all putative macrophages etc) are compared on a per cell basis over time, for example, at a first and second time point. Here, the comparison relates to the change in the cell population(s), but due to the nature of the cell movements and/or imaging location, it is not always possible to capture the same exact cells and as such, such cells are referred to herein as being of “corresponding cell type”.

In some embodiments, the condition or disease state is chronic inflammation in the eye(s), for example, caused by an allergic condition.

In some embodiments, the method further comprises the steps of: providing an agent to the subject before determining the morphology, or number, or density, or dynamic behaviour of one or more immune cells at one or more locations within a focal plane (x, y) and/or at one or more focal depths (z) in corneal tissue of the subject over time. The agent may be selected from at least one of a group comprising: an environmental agent; an exogenous agent; a neurostimulatory agent; a dietary supplement (e.g., fish oil), and a therapeutic agent. The therapeutic agent may be an immunomodulator.

In other embodiments, the method further comprises the steps of: providing a device (e.g., a medical device such as a contact lens) to the subject (e.g., to the eye of the subject) before determining the morphology, or number, or density, or dynamic behaviour of one or more immune cells at one or more locations within a focal plane (x, y) and/or at one or more focal depths (z) in corneal tissue of the subject over time.

In some embodiments, the method comprises determining the dynamic behaviour of one or more putative T cells in the epithelium of the corneal tissue, wherein a higher motility of the putative T cells at about the corneal whorl region compared to the reference level is indicative of chronic inflammation in the eye(s) caused by, for example, an allergic condition. The putative T cells may have one or more of the following motility parameters: higher instantaneous speed and lower arrest co-efficient compared to the reference level.

For example, individual putative corneal epithelial T cells at about the corneal whorl region of the eye(s) with chronic inflammation caused by, for example, an allergic condition may have on average about a 0.1 to about 0.5 μm/min higher mean instantaneous speed than a corresponding cell type of healthy control eye(s). In another or further example, in a subject, putative corneal epithelial T cells at about the corneal whorl region of the eye(s) with chronic inflammation caused by, for example, an allergic condition have, on average, a mean instantaneous cell speed of more than about 1.3 μm/min.

In other or additional examples, individual putative corneal epithelial T cells at about the corneal whorl region of the eye(s) with chronic inflammation caused by, for example, an allergic condition may have on average about a 0.05 to about 0.3 lower arrest coefficient than a corresponding cell type of healthy control eye(s). In another or further example, in a subject, putative corneal epithelial T cells at about the corneal whorl region of the eye(s) with chronic inflammation caused by, for example, an allergic condition have, on average, an arrest coefficient of lower than about 0.85.

In some embodiments, the method comprises determining the density of one or more putative dendritic cells in the epithelium of the corneal tissue, wherein a lower density of the putative dendritic cells at about the corneal whorl region compared to the reference level is indicative of chronic inflammation in the eye(s) caused by, for example, an allergic condition.

For example, individual putative corneal epithelial dendritic cells at about the corneal whorl region of the eye(s) with chronic inflammation caused by, for example, an allergic condition may have on average about a 1 to about 10 cells/mm² lower mean density than a corresponding cell type of healthy control eye(s). In another or further example, in a subject, putative corneal epithelial dendritic cells at about the corneal whorl region of the eye(s) with chronic inflammation caused by, for example, an allergic condition have a mean density of less than about 2 cells/mm².

In some embodiments, the method comprises determining the dynamic behaviour of one or more putative dendritic cells in the epithelium of the corneal tissue, wherein a lower dynamic activity of the putative dendritic cells at about the corneal whorl region compared to the dynamic activity of a corresponding cell type of healthy control eye(s) is indicative of chronic inflammation in the eye(s) caused by, for example, an allergic condition. The putative dendritic cells may have a lower dSEARCH index per minute (normalized dSEARCH index).

For example, individual putative corneal epithelial dendritic cells at about the corneal whorl region of the eye(s) with chronic inflammation caused by, for example, an allergic condition may have on average about a 0.5 to about 2 lower dSEARCH index per minute than a corresponding cell type of healthy control eye(s). In another or further example, in a subject, putative corneal epithelial dendritic cells at about the corneal whorl region of the eye(s) with chronic inflammation caused by, for example, an allergic condition have, on average, a dSEARCH index per minute of less than about 1.1.

In some embodiments, the method comprises determining the density of one or more putative macrophage cells in the stroma of the corneal tissue, wherein a lower density of the putative macrophage cells at about the corneal whorl region compared to the reference level is indicative of chronic inflammation in the eye(s) caused by, for example, an allergic condition.

For example, individual putative corneal stromal macrophage cells at about the corneal whorl region of the eye(s) with chronic inflammation caused by, for example, an allergic condition may on average have about a 5 to about 35 cells/mm² lower mean density than a corresponding cell type of healthy control eye(s). In another or further example, in a subject, putative corneal stromal macrophage cells at about the corneal whorl region of the eye(s) with chronic inflammation caused by, for example, an allergic condition have a mean density of less than about 70 cells/mm².

In some embodiments, the method comprises determining the morphology of one or more putative macrophage cells in the stroma of the corneal tissue, wherein putative macrophage cells at about the corneal whorl region having one or more of the following parameters: a deviation in cell area or cell perimeter compared to the reference level, are indicative of chronic inflammation in the eye(s) caused by, for example, an allergic condition. For example, a deviation of about 25% in cell area, or a deviation of about 15% in cell perimeter of putative corneal stromal macrophage cells at about the corneal whorl region compared to a corresponding cell type of healthy control eye(s), is indicative of chronic inflammation in the eye(s) caused by, for example, an allergic condition.

Putative corneal stromal macrophage cells at about the corneal whorl region of the eye(s) with chronic inflammation caused by, for example, an allergic condition may have one or more of the following parameters: a smaller cell area or a smaller cell perimeter compared to the reference level. For example, individual putative corneal stromal macrophage cells at about the corneal whorl region of the eye(s) with chronic inflammation caused by, for example, an allergic condition may have on average about a 50 to about 100 μm² smaller cell area than a corresponding cell type of healthy control eye(s). In another or further example, in a subject, putative corneal stromal macrophage cells at about the corneal whorl region of the eye(s) with chronic inflammation caused by, for example, an allergic condition have, on average, a cell area of less than about 250 μm².

In other or additional examples, individual putative corneal stromal macrophage cells at about the corneal whorl region of the eye(s) with chronic inflammation caused by, for example, an allergic condition may on average have about a 15 to about 25 μm smaller cell perimeter than a corresponding cell type of healthy control eye(s). In another or further example, in a subject, putative corneal stromal macrophage cells at about the corneal whorl region of the eye(s) with chronic inflammation caused by, for example, an allergic condition have, on average, a cell perimeter of less than about 100 μm.

In other embodiments, the condition or disease state is acute inflammation in the eye(s) caused by contact lens wear.

In some embodiments, the method comprises determining the density of one or more putative T cells in the epithelium of the corneal tissue, wherein a lower density of the putative T cells at about the corneal whorl region compared to the reference level is indicative of acute inflammation in the eye(s) caused by contact lens wear. For example, putative corneal epithelial T cells at about the corneal whorl region of the eye(s) with acute inflammation caused by contact lens wear may have on average about an 8 to about 16 cells/mm² lower mean density than a corresponding cell type of the eye(s) without acute inflammation (e.g., baseline level, prior to contact lens wear). In another or further example, in a subject, putative corneal epithelial T cells at about the corneal whorl region of the eye(s) with acute inflammation caused by contact lens wear have an about 50% less mean density than a corresponding cell type of the eye(s) without acute inflammation.

In some embodiments, the method comprises determining the dynamic behaviour of one or more putative T cells in the epithelium of the corneal tissue, wherein a higher motility of the putative T cells at about the corneal whorl region compared to the reference level is indicative of acute inflammation in the eye(s) caused by contact lens wear. The putative T cells at about the corneal whorl region may have one or more of the following motility parameters: a higher mean instantaneous speed, a lower arrest co-efficient, higher displacement speed, higher meandering index, and higher linearity of forward progression compared to the reference level.

For example, individual putative corneal epithelial T cells at about the corneal whorl region of eye(s) with acute inflammation caused by contact lens wear may have on average about a 0.3 to about 0.7 μm/min higher mean instantaneous speed than a corresponding cell type of the eye(s) without acute inflammation (e.g., baseline level, prior to contact lens wear). In another or further example, in a subject, putative corneal epithelial T cells at about the corneal whorl region of the eye(s) with acute inflammation caused by contact lens wear have, on average, a mean instantaneous speed of more than about 2.3 μm/min.

In other or additional examples, individual putative corneal epithelial T cells at about the corneal whorl region of the eye(s) with acute inflammation caused by contact lens wear may have on average about a 0.15 to about 0.25 lower arrest coefficient than cells of the eye(s) without acute inflammation (e.g., baseline level, prior to contact lens wear). In another or further example, in a subject, putative corneal epithelial T cells of the eye(s) with acute inflammation caused by contact lens wear have, on average, an arrest coefficient of lower than about 0.55.

In other or additional examples, individual putative corneal epithelial T cells at about the corneal whorl region of the eye(s) with acute inflammation caused by contact lens wear may have on average about a 0.7 to about 0.9 μm/min higher mean displacement speed than a corresponding cell type of the eye(s) without acute inflammation (e.g., baseline level, prior to contact lens wear). In another or further example, in a subject, putative corneal epithelial T cells at about the corneal whorl region of the eye(s) with acute inflammation caused by contact lens wear have, on average, an increase in the mean displacement speed of at least about 25% than a corresponding cell type of the eye(s) without acute inflammation (e.g., baseline level, prior to contact lens wear), and/or, on average, a mean displacement speed of more than about 1.3 μm/min.

In other or additional examples, individual putative corneal epithelial T cells at about the corneal whorl region of the eye(s) with acute inflammation caused by contact lens wear may have on average about a 0.1 to about 0.3 higher meandering index than cells of the eye(s) without acute inflammation (e.g., prior to contact lens wear). In another or further example, in a subject, putative corneal epithelial T cells at about the corneal whorl region of the eye(s) with acute inflammation caused by contact lens wear have an average increase in the meandering index of at least about 30% than a corresponding cell type of the eye(s) without acute inflammation (e.g., baseline level, prior to contact lens wear), and, on average, a meandering index of more than about 0.55.

In other or additional examples, individual putative corneal epithelial T cells at about the corneal whorl region of the eye(s) with acute inflammation caused by contact lens wear may have on average about a 0.15 to about 0.35 higher linearity of forward progression than a corresponding cell type of the eye(s) without acute inflammation (e.g., baseline level, prior to contact lens wear). In another or further example, in a subject, putative corneal epithelial T cells at about the corneal whorl region of the eye(s) with acute inflammation caused by contact lens wear have, on average, an increase in the linearity of forward progression of at least about 8% than a corresponding cell type of the eye(s) without acute inflammation (e.g., baseline level, prior to contact lens wear), and/or an average linearity of forward progression of more than about 0.5.

In some embodiments, the method comprises determining the dynamic behaviour of one or more putative dendritic cells in the epithelium of the corneal tissue, wherein lower dynamic activity of the putative dendritic cells at about the corneal whorl region compared to the reference level is indicative of acute inflammation in the eye(s) caused by contact lens wear. The putative dendritic cells may have a lower dSEARCH index per minute (normalized dSEARCH index) compared to the reference level. For example, individual putative corneal epithelial dendritic cells at about the whorl region of the eye(s) with acute inflammation caused by contact lens wear may have on average about a 0.15 to about 1.5 lower dSEARCH index per minute than a corresponding cell type (e.g., a matched cell) of the eye(s) without acute inflammation (e.g., baseline level, prior to contact lens wear). In another or further example, in a subject, putative corneal epithelial dendritic cells at about the whorl region of the eye(s) with acute inflammation caused by contact lens wear have, on average, a decrease in dSEARCH index per minute of at least about 10%, for example, to about 35%, than a corresponding cell type of the eye(s) without acute inflammation (e.g., baseline level, prior to contact lens wear), and/or, on average, a dSEARCH index per minute of less than about 2.5.

In some embodiments, the method comprises determining the morphology of putative macrophage cells in the stroma of the corneal tissue, wherein putative macrophage cells at about the corneal whorl region having one or more of the following parameters: a larger cell area or cell perimeter, lower solidity, or a reduced circularity index compared to the reference level, may be indicative of acute inflammation in the eye(s) caused by contact lens wear.

For example, individual putative corneal stromal macrophage cells at about the whorl region of the eye(s) with acute inflammation caused by contact lens wear may have on average about a 35 to about 65 μm² larger cell area than a corresponding cell type (e.g., a matched cell) of the eye(s) without acute inflammation (e.g., baseline level, prior to contact lens wear). In another or further example, in a subject, putative corneal stromal macrophage cells at about the whorl region of the eye(s) with acute inflammation caused by contact lens wear have, on average, a cell area of more than about 270 μm².

In other or additional examples, individual putative corneal stromal macrophage cells at about the whorl region of the eye(s) with acute inflammation caused by contact lens wear may have about a 14 to about 26 μm larger cell perimeter than a corresponding cell type (e.g., a matched cell) of the eye(s) without acute inflammation (e.g., prior to contact lens wear). In another or further example, in a subject, putative corneal stromal macrophage cells at about the whorl region of the eye(s) with acute inflammation caused by contact lens wear have, on average, a cell perimeter of greater than at least about 10%, for example up to about 45%, than a corresponding cell type of the eye(s) without acute inflammation (e.g., baseline level, prior to contact lens wear), or, on average, a cell perimeter of more than about 85 μm.

In other or additional examples, individual putative corneal stromal macrophage cells at about the whorl region of the eyes with acute inflammation caused by contact lens wear may have on average about a 0.05 to about 0.2 lower solidity index compared to a corresponding cell type (e.g., a matched cell) of the eye(s) without acute inflammation (e.g., prior to contact lens wear). In another or further example, in a subject, putative corneal stromal macrophage cells at about the whorl region of the eye(s) with acute inflammation caused by contact lens wear have, on average, a solidity index of at least about 8% lower, for example up to at least about 25% lower, than a corresponding cell type of the eye(s) without acute inflammation (e.g., baseline level, prior to contact lens wear), or, on average, a solidity index of less than about 0.75.

In other or additional examples, individual putative corneal stromal macrophage cells at about the whorl region of the eye(s) with acute inflammation caused by contact lens wear may have on average about a 0.05 to about 0.20 lower average circularity compared to a corresponding cell type (e.g., a matched cell) of the eye(s) without acute inflammation (e.g., baseline level, prior to contact lens wear). In another or further example, in a subject, putative corneal stromal macrophage cells at about the whorl region of the eyes with acute inflammation caused by contact lens wear have, on average, a circularity of at least about 15% lower, for example up to at least about 40% lower, than a corresponding cell type of the eye(s) without acute inflammation (e.g., baseline level, prior to contact lens wear), or on average, a circularity of less than about 0.5.

In other embodiments, the method is used to detect changes in cell dynamic behaviours associated with changes in immune status of a subject infected with HIV. For example, the method may be used to determine the dynamic behaviour of one or more putative T cells in the epithelium of the corneal tissue, wherein a higher motility of the putative T cells at about the corneal peripheral region compared to the reference level is associated with HIV infection. In a further example, the putative T cells at about the corneal peripheral region may have one or more of the following motility parameters: a higher mean trajectory speed, and a higher percentage of mobile T cells.

In the above examples, where the data relating to a putative immune cell is referred to as being “individual”, the values given are based on a per cell level analysis where each cell, captured from different participants, is considered as an independent sample. These comparative quantitative data are for populations of cells from the eyes of multiple subjects, combined together.

In the above examples, where the data relating to a putative immune cell is referred to as being “in a subject”, the values given are based on a per participant level analysis of the same data (where each subject is represented by a single data point, calculated as the average value from multiple cells from that person (e.g., from 1 to 15 cells)). These comparative quantitative data are for people, derived from averaging data from multiple individual cells within an individual subject's eye.

In the above examples, where the putative immune cell is referred to as a “matched cell”, the values given are based on a per cell level analysis, where each cell is represented by a single data point across two observation periods, before and after exposure to an agent, for example, before and after contact lens wear. This comparative quantitative paired data is for cells from multiple eyes, combined together.

In some embodiments, the method further comprises selecting a treatment or modifying a treatment, based on the identification of a condition or disease state in a subject.

In some embodiments, the treatment is for preventing, inhibiting, or reducing chronic inflammation in the eye and is selected from, for example, from administration of a therapeutic agent for neuropathy or ocular pain or discomfort, an anti-inflammatory agent, a wetting agent, an immunomodulatory agent, a tear composition modifying agent, a viscoelastic agent, a different contact lens, a different contact lens disinfection system, a tear retention agent (e.g., punctal plugs), a tear stimulatory agent (e.g., neurostimulation or secretagogue), a dry eye treatment, an ocular anti-allergy agent, an environmental modification (e.g., use of a humidifier, avoidance of air drafts, etc.) and combinations thereof.

In another aspect, the present disclosure provides a method of diagnosing a condition or disease state in a subject, or predicting the likelihood of a subject developing a condition or disease state, the method comprising the steps of: a) determining the morphology, or number, or density, or dynamic behaviour of one or more immune cells at one or more locations within the focal plane (x, y) and/or at one or more focal depths (z) in corneal tissue of the subject over time; b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a subject with the condition or disease state, or a reference level indicative of a subject likely to develop the condition or disease state; and c) optionally, administering a treatment or preventative therapy to the subject when the subject is diagnosed with the condition or disease state, or deemed likely to develop the condition or disease state.

In another aspect, the present disclosure provides a method of treating a condition or disease state in a subject, the method comprising the steps of: a) determining the morphology, or number, or density, or dynamic behaviour of one or more immune cells at one or more locations within a focal plane (x, y) and/or at one or more focal depths (z) in corneal tissue of the subject over time; b) comparing the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject; and c) selecting a treatment or modifying a treatment, based on the identification of a condition or disease state in a subject.

In another aspect, the present disclosure provides a method of monitoring treatment efficacy of a condition or disease state in a subject, the method comprising the steps of: a) determining the morphology, or number, or density, or dynamic behaviour of one or more immune cells at one or more locations within the focal plane (x, y) and/or at one or more focal depths (z) in corneal tissue of the subject over time; b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a responder or non-responder to treatment or to a baseline level before initiation of treatment; and c) optionally, continuing treatment when the subject is determined to be a responder; or switching the treatment when the subject is determined to be a non-responder, wherein switching the treatment comprises increasing a dose of the treatment administered, adding one or more of agents to the treatment regimen, discontinuing the treatment and initiating a different treatment regimen.

In another aspect, the present disclosure provides a system for imaging a biological tissue over time, the system including: a communication interface configured to provide microscopy images of the biological tissue acquired over a time period; a processor; and a computer-readable medium communicatively coupled to the processor and storing computer-executable instructions that, when executed by the processor, cause the processor to: provide microscopy images of the biological tissue acquired over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identify at least one microscopy image acquired at the first time point and the second time point; and produce a sequence of the identified microscopy images, thereby imaging the biological tissue over the time period.

In another aspect, the present disclosure provides a system for imaging a biological tissue over time, the system including: a communication interface configured to provide microscopy images of the biological tissue acquired over a time period; a processor; and a computer-readable medium communicatively coupled to the processor and storing computer-executable instructions that, when executed by the processor, cause the processor to: provide microscopy images of the biological tissue acquired over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue, identify at least one microscopy image acquired at the first time point and the second time point; and produce a sequence of the identified microscopy images, thereby imaging the biological tissue over the time period.

In another aspect, the present disclosure provides a system for imaging a biological tissue over time, the system including: a communication interface configured to provide microscopy images of the biological tissue acquired over a time period; a processor; and a computer-readable medium communicatively coupled to the processor and storing computer-executable instructions that, when executed by the processor, cause the processor to: acquire microscopy images of the biological tissue over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identify at least one microscopy image acquired at the first time point and the second time point; and produce a sequence of the identified microscopy images, thereby imaging the biological tissue over the time period.

In another aspect, the present disclosure provides a system for imaging a biological tissue over time, the system including: a communication interface configured to provide microscopy images of the biological tissue acquired over a time period; a processor; and a computer-readable medium communicatively coupled to the processor and storing computer-executable instructions that, when executed by the processor, cause the processor to: acquire microscopy images of the biological tissue over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue; identify at least one microscopy image acquired at the first time point and the second time point; and produce a sequence of the identified microscopy images, thereby imaging the biological tissue over the time period.

In another aspect, the present disclosure provides a system for identifying a condition or disease state in a subject, the system including: a communication interface; a processor; and a computer-readable medium communicatively coupled to the processor and storing computer-executable instructions that, when executed by the processor, cause the processor to: a) determine the morphology, or number, or density, or dynamic behaviour of one or more immune cells at one or more locations within a focal plane (x, y) and/or at one or more focal depths (z) in corneal tissue of the subject over time; and b) compare the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject.

In another aspect, the present disclosure provides a system for selecting or modifying a treatment for a condition or disease state in a subject, the system including: a communication interface; a processor; and a computer-readable medium communicatively coupled to the processor and storing computer-executable instructions that, when executed by the processor, cause the processor to: a) determine the morphology, or number, or density, or dynamic behaviour of one or more immune cells at one or more locations within a focal plane (x, y) and/or at one or more focal depths (z) in corneal tissue of the subject over time; b) compare the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject; and c) select a treatment or modifying a treatment, based on the identification of a condition or disease state in a subject.

In another aspect, the present disclosure provides a system for monitoring treatment efficacy of a condition or disease state in a subject, the system including: a communication interface; a processor; and a computer-readable medium communicatively coupled to the processor and storing computer-executable instructions that, when executed by the processor, cause the processor to: a) determine the morphology, or number, or density, or dynamic behaviour of one or more immune cells at one or more locations within the focal plane (x, y) and/or at one or more focal depths (z) in corneal tissue of the subject over time; b) correlate the morphology, or number, or density, or dynamic behaviour of the one or more immune cells at the one or more locations within the focal plane (x, y) and/or at the one or more focal depths (z) in the corneal tissue of the subject over time to a reference level indicative of a responder or non-responder to treatment or to a baseline level before initiation of treatment; and c) optionally, continue treatment when the subject is determined to be a responder; or switch the treatment when the subject is determined to be a non-responder, wherein switching the treatment comprises increasing a dose of the treatment administered, adding one or more of agents to the treatment regimen, discontinuing the treatment and initiating a different treatment regimen.

In another aspect, the present disclosure provides a system for diagnosing a condition or disease state in a subject, or predicting the likelihood of a subject developing a condition or disease state, the system including: a communication interface; a processor; and a computer-readable medium communicatively coupled to the processor and storing computer-executable instructions that, when executed by the processor, cause the processor to: a) determine the morphology, or number, or density, or dynamic behaviour of one or more immune cells at one or more locations within the focal plane (x, y) and/or at one or more focal depths (z) in corneal tissue of the subject over time; b) correlate the morphology, or number, or density, or dynamic behaviour of the one or more immune cells at the one or more locations within the focal plane (x, y) and/or at the one or more focal depths (z) in the corneal tissue of the subject over time to a reference level indicative of a subject with the condition or disease state, or a reference level indicative of a subject likely to develop the condition or disease state; and c) optionally, administer a treatment or preventative therapy to the subject when the subject is diagnosed with the condition or disease state, or deemed likely to develop the condition or disease state.

In another aspect, the present disclosure provides a non-transitory computer-readable medium for imaging a biological tissue over time, including instructions stored thereon, that when executed on a processor, perform the steps of: providing microscopy images of the biological tissue acquired over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identifying at least one microscopy image acquired at the first time point and the second time point; and producing a sequence of the identified microscopy images, thereby imaging the biological tissue over the time period.

In another aspect, the present disclosure provides a non-transitory computer-readable medium for imaging a biological tissue over time, including instructions stored thereon, that when executed on a processor, perform the steps of: providing microscopy images of the biological tissue acquired over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue, identifying at least one microscopy image acquired at the first time point and the second time point; and producing a sequence of the identified microscopy images, thereby imaging the biological tissue over the time period.

In another aspect, the present disclosure provides a non-transitory computer-readable medium for imaging a biological tissue over time, including instructions stored thereon, that when executed on a processor, perform the steps of: acquiring microscopy images of the biological tissue over a time period, comprising: one or more microscopy images acquired at a first time point over the time period, wherein the one or more microscopy images are acquired at a first location within a focal plane (x, y) of the biological tissue and at a focal depth (z) of the biological tissue; and one or more microscopy images acquired at a second time point over the time period, wherein the one or more microscopy images are acquired at the first location within the focal plane (x, y) of the biological tissue and at the same or a different focal depth (z) of the biological tissue; identifying at least one microscopy image acquired at the first time point and the second time point; and producing a sequence of the identified microscopy images, thereby imaging the biological tissue over the time period.

In another aspect, the present disclosure provides a non-transitory computer-readable medium for imaging a biological tissue over time, including instructions stored thereon, that when executed on a processor, perform the steps of: acquiring microscopy images of the biological tissue over a time period, comprising: a plurality of microscopy images acquired at a first time point over the time period, wherein the plurality of microscopy images is a series of repeated images acquired at a first location and at a second location within a focal plane (x, y) of the biological tissue; and a plurality of microscopy images acquired at a second time point over the time period, wherein the plurality of microscopy images is a series of repeated images acquired at the first location and at the second location within the focal plane (x, y) of the biological tissue; identifying at least one microscopy image acquired at the first time point and the second time point; and producing a sequence of the identified microscopy images, thereby imaging the biological tissue over the time period.

In another aspect, the present disclosure provides a non-transitory computer-readable medium for identifying a condition or disease state in a subject, including instructions stored thereon, that when executed on a processor, perform the steps of: a) determining the morphology, or number, or density, or dynamic behaviour of one or more immune cells at one or more locations within a focal plane (x, y) and/or at one or more focal depths (z) in corneal tissue of the subject over time; and b) comparing the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject.

In another aspect, the present disclosure provides a non-transitory computer-readable medium for selecting a treatment for a condition or disease state in a subject, including instructions stored thereon, that when executed on a processor, perform the steps of: a) determining the morphology, or number, or density, or dynamic behaviour of one or more immune cells at one or more locations within a focal plane (x, y) and/or at one or more focal depths (z) in corneal tissue of the subject over time; b) comparing the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject; and c) selecting a treatment or modifying a treatment, based on the identification of a condition or disease state in a subject.

In another aspect, the present disclosure provides a non-transitory computer-readable medium for monitoring treatment efficacy of a condition or disease state in a subject, including instructions stored thereon, that when executed on a processor, perform the steps of: a) determining the morphology, or number, or density, or dynamic behaviour of one or more immune cells at one or more locations within the focal plane (x, y) and/or at one or more focal depths (z) in corneal tissue of the subject over time; b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a responder or non-responder to treatment or to a baseline level before initiation of treatment; and c) optionally, continuing treatment when the subject is determined to be a responder; or switching the treatment when the subject is determined to be a non-responder, wherein switching the treatment comprises increasing a dose of the treatment administered, adding one or more of agents to the treatment regimen, discontinuing the treatment and initiating a different treatment regimen.

In another aspect, the present disclosure provides a non-transitory computer-readable medium for diagnosing a condition or disease state in a subject, or predicting the likelihood of a subject developing a condition or disease state, including instructions stored thereon, that when executed on a processor, perform the steps of: a) determining the morphology, or number, or density, or dynamic behaviour of one or more immune cells at one or more locations within the focal plane (x, y) and/or at one or more focal depths (z) in corneal tissue of the subject over time; b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a subject with the condition or disease state, or a reference level indicative of a subject likely to develop the condition or disease state; and c) optionally, administering a treatment or preventative therapy to the subject when the subject is diagnosed with the condition or disease state, or deemed likely to develop the condition or disease state.

In another aspect, the present disclosure provides an image processing method for a scanning instrument for scanning biological tissue the method including: receiving two or more images of biological tissue; selecting a first reference image from the two or more images; translating the two or more images to align with the first reference image; applying a first strip-based image registration on the two or more images to thereby determine a degree of intra-image warp between the two or more images; based on the degree of intra-image warp, automatically selecting a second reference image; and averaging and further registering the two or more images.

A second strip-based image registration may be applied to align the two or more image with the second reference image. In an embodiment, a third strip-based image registration may be applied.

The scanning instrument may include one or more of an in vivo confocal microscope, a scanning laser ophthalmoscope, an optical coherence tomography instrument, or other scan-based instruments applied to a moving tissue (for example, the eye). Preferably, the second reference image has reduced distortion compared to remaining images.

Preferably, the averaging and further registering the two or more images includes applying a full field translation on the two or more images; applying a further registration to the two or more images; and de-warping the two or more images. The registration may include, for example affine registration (or any other suitable registration approach) as would be appreciated by the skilled person.

In another aspect, the present disclosure provides an image processing system for a scanning instrument for scanning biological tissue the system including a communication interface configured to provide microscopy images of the biological tissue; a processor; and a computer-readable medium communicatively coupled to the processor and storing computer-executable instructions that, when executed by the processor, cause the processor to: receive two or more images of biological tissue; select a first reference image from the two or more images; translate the two or more images to align with the first reference image; apply a first strip-based image registration on the two or more images to thereby determine a degree of intra-image warp between the two or more images; automatically select a second reference image based on the degree of intra-image warp; and average and further register the two or more images.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

The disclosure will now be described in greater detail with reference to the accompanying drawings in which like features are represented by like numerals. It is to be understood that the embodiments shown are examples only and are not to be taken as limiting the scope of the disclosure as defined in the provisional claims appended hereto.

FIG. 1 illustrates a laser-scanning, in vivo confocal microscopy (IVCM) setup with the Heidelberg HRT-3 with Rostock Corneal Module, to acquire images of the human cornea, according to some embodiments of the disclosure.

FIG. 2 is a schematic diagram illustrating the laser-scanning IVCM setup of FIG. 1 shown diagrammatically in histological cross-section (not to scale), showing a volume scan across a depth of the cornea, according to some embodiments of the disclosure.

FIG. 3 is a diagrammatic view of the corneal tissue of a subject along the broken line in FIG. 2 representing the central axis of a subject's eye, illustrating 2D en face regions in a focal plane (x, y) showing the nerve pattern representing the whorl region and a location for imaging the whorl in the paracentral cornea, according to some embodiments of the disclosure.

FIG. 4 is a schematic diagram showing regions of the cornea comprising the central cornea, the paracentral cornea and the peripheral cornea, where the whorl region is located in the inferior paracentral corneal region, according to some embodiments of the disclosure.

FIGS. 5 and 6 illustrate a single en face microscopy image identified from the volume scan of FIG. 2 at the depth d₁ of the corneal epithelium (FIG. 5) and at the depth d₂ of the corneal stroma (FIG. 6), according to some embodiments of the disclosure.

FIG. 7 is a schematic diagram of a cornea of the subject showing one location for volume imaging, according to some embodiments of the disclosure.

FIG. 8 is a schematic diagram illustrating volume imaging of the cornea of the subject at two depths, namely at the level of the epithelium and the stroma, and at the same location in a focal plane (x, y) of tissue, and creation of time-lapsed videos and colour time-lapsed merges of the images, according to some embodiments of the disclosure.

FIG. 9 is a flow chart of a method for imaging a biological tissue over time, relating to providing microscopy images at a first location and either at the same or a different focal depth (z) of the biological tissue, according to some embodiments of the disclosure.

FIG. 10 is a flow chart showing additional steps in the method of FIG. 9 relating to providing additional microscopy images at a second location and either at the same or a different focal depth (z) of the biological tissue, according to some embodiments of the disclosure.

FIG. 11 is a flow chart showing additional steps in the method of FIG. 9 relating to providing microscopy images acquired at one or more subsequent time point(s) at the first location, according to some embodiments of the disclosure.

FIG. 12 is a flow chart showing additional steps in the method of FIG. 9 relating to analysing the identified microscopy images to locate one or more anatomical landmarks and registering the images to the landmarks, according to some embodiments of the disclosure.

FIG. 13 is a flow chart showing additional steps in the method of FIG. 9 relating to producing a time-lapsed video and a combined image from the sequence of identified microscopy images, according to some embodiments of the disclosure.

FIG. 14 is a flow chart showing additional steps in the method of FIG. 9 relating to determining the presence of at least one object, e.g. a cell, in one or more of the identified microscopy images, monitoring dynamic behaviour of the object and then optionally tracking movement of the object across a range of focal depths (z) and/or when the object is a cell, determining cell type, according to some embodiments of the disclosure.

FIG. 15 is a schematic diagram of a cornea of the subject showing a first location and a second location for volume imaging, according to some embodiments of the disclosure.

FIG. 16 is an example of a plurality of microscopy images acquired across a predetermined range of focal depths (z) of the cornea at a first location at one time point, according to some embodiments of the disclosure.

FIG. 17 is a series of microscopy images acquired across a depth of the cornea at a first location (top row), namely the paracentral whorl, and a second location (bottom row), namely the peripheral cornea, at five time points, and identifying at least two microscopy images at each time point to produce a sequence of images, according to some embodiments of the disclosure.

FIG. 18 is another method for imaging a biological tissue over time, relating to providing microscopy images comprising a plurality of microscopy images acquired at a first time point and a second time point and at each of a first and second location within a focal plane (x, y) of the tissue, according to some embodiments of the disclosure.

FIG. 19 is a flow chart showing additional steps in the method of FIG. 18 relating to identifying at least one microscopy image at each of the first and second locations, producing first and second sequences of the images, and producing a time-lapsed video and/or and a colour time-lapsed merge being a 2D representation of the sequence of images, according to some embodiments of the disclosure.

FIG. 20 is a schematic diagram of a cornea of the subject showing a first location and a second location for sequential imaging, according to some embodiments of the disclosure.

FIGS. 21 and 22 illustrate a series of sequential microscopy images acquired at two locations, namely the paracentral cornea (FIG. 21) and the peripheral cornea (FIG. 22), identifying one image for analysis, and determining the presence of at least one cell in the image, being a motile “T cell” (FIG. 21) and a sessile “dendritic cell” (FIG. 22), according to some embodiments of the disclosure. Three cells are identified by circles in FIG. 21 and four cells are identified by circles in FIG. 22.

FIG. 23 is a series of microscopy images acquired sequentially at each of two locations, the paracentral whorl epithelium and the peripheral corneal epithelium, at five time points, and identifying at least one microscopy image for each time point to produce the image sequence, according to some embodiments of the disclosure.

FIG. 24 is a flow chart showing additional steps in the method of FIG. 9 or FIG. 18 relating to determining the presence of at least two objects and assessing an object-to-object interaction, according to some embodiments of the disclosure.

FIG. 25 is a flow chart showing additional steps in the method of FIG. 9 or FIG. 18 relating to determining the presence of at least one nerve and assessing an object-to-nerve interaction, according to some embodiments of the disclosure.

FIG. 26 is a flow chart showing additional steps in the method of FIG. 9 or FIG. 18 relating to acquiring additional microscopy images in a subsequent acquisition at a specified interval from the first acquisition, according to some embodiments of the disclosure.

FIG. 27 is a flow chart showing additional steps in the method of FIG. 26 relating to identifying a condition or disease state based on a change in immune status of the biological tissue, according to some embodiments of the disclosure.

FIG. 28 is a flow chart showing additional steps in the method of FIGS. 26 and 27 relating to assessing an effect of an agent on the biological tissue, according to some embodiments of the disclosure.

FIG. 29 is a flow chart showing additional steps in the method of FIGS. 26 and 27 relating to assessing an effect of a medical device or medical therapy on the biological tissue, according to some embodiments of the disclosure.

FIG. 30 is a flow chart illustrating another method for imaging a biological tissue over time, which is similar to the method of FIG. 9 except the first step involves acquiring the microscopy images, according to some embodiments of the disclosure.

FIG. 31 is a flow chart illustrating another method for imaging a biological tissue over time, which is similar to the method of FIG. 18 except the first step involves acquiring the microscopy images, according to some embodiments of the disclosure.

FIG. 32 is a flow chart illustrating another method for imaging a biological tissue over time where the steps are performed by a processor and the microscopy images are acquired at the same or a different focal depth (z) of the biological tissue, according to some embodiments of the disclosure.

FIG. 33 is a flow chart illustrating another method for imaging a biological tissue over time where the steps are performed by a processor and the microscopy images are acquired at two or more locations, according to some embodiments of the disclosure.

FIG. 34 is a schematic diagram illustrating a system for imaging a biological tissue configured to perform the methods of FIG. 32 or FIG. 33 by a processor, according to some embodiments of the disclosure.

FIGS. 35-38 relate to Example 1A where immune cell subsets defined using the new IVCM imaging technique in healthy human corneas are comparable to phenotyped mouse corneal immune cell subtypes.

FIG. 35 illustrates sequential time-lapsed images acquired over 35 minutes, and a colour time-lapsed merge, captured using the new IVCM technique to reveal the dynamic behaviours of putative [i]T cells (orange arrow tracks a single cell) that are motile in the human corneal epithelium; [ii] Dendritic cells (DCs) showing dSEARCH behaviours (yellow triangle is stationary, and highlights active dendrite retraction); and [iii] Macrophages in the corneal stroma that crawl between keratocyte nuclei (pink arrow); the green arrow highlights the alterations to macrophage cell position and morphology. Scale bar applies to all images.

FIG. 36 illustrates sequential time-lapsed images, and colour time-lapsed merges for each of: [i] GFP+ T cells in the corneal epithelium of a mouse after ocular herpes simplex virus infection, imaged in vivo using intravital 2-photon microscopy; [ii] CD11c-eYFP+ mouse corneal epithelial DCs, imaged ex vivo; [iii] CX3CR1-GFP+ mouse corneal macrophages, imaged ex vivo. Scale bar applies to all images. Cell parameters were quantified from mouse in vivo immunofluorescent images and show cell morphological characteristics that parallel the human corneal cell subsets imaged using the new IVCM imaging technique.

FIG. 37 illustrates a 3D scatterplot showing the cell field area (μm)×cell area (μm)×circularity parameters for the three immune cell subsets in healthy human corneas, quantified from the new IVCM images.

FIG. 38 illustrates an equivalent 3D scatterplot to FIG. 37, for phenotyped mouse corneal immune cells.

FIGS. 39-51 relate to Example 1B where putative T cells are shown to dynamically patrol the corneal epithelium and are responsive to inflammatory stimuli.

FIG. 39 illustrates sequential time-lapsed images acquired over 40 minutes, with four individual cells noted (triangles labelled 1 to 4). Individual cell tracks and a colour time-lapsed merge are also shown. Scale bar applies to all images.

FIG. 40 is a chart illustrating that putative T cell densities were similar in the central and paracentral (whorl) corneal regions, and in control (n=16 people), untreated allergy (n=10 people) and treated allergy (n=4 people) eyes (p>0.05 for all comparisons).

FIG. 41 is a chart illustrating that putative T cell density in the central and paracentral (whorl) corneal regions were moderately correlated in individual subjects (R²=0.43, p<0.0001).

FIG. 42 is a chart illustrating that the mean instantaneous cell speed of putative T cells in the corneal epithelium was significantly higher in individuals with untreated allergy (n=41 cells), relative to speeds evident in both healthy control (n=65 cells) and treated allergy (n=24 cells) eyes. **p<0.01.

FIG. 43 is a chart illustrating that the mean arrest coefficient of putative T cells in the corneal epithelium was significantly lower in individuals with untreated allergy, relative to both healthy control and treated allergy eyes. **p<0.01.

FIGS. 44 to 49 are charts illustrating various morphological and/or dynamic parameters of putative T cells in the corneal epithelium after short-term contact lens (CL) wear. There were significantly fewer cells in the paracentral (whorl) region relative to pre-CL levels (FIG. 44). Post-CL wear cells showed faster mean instantaneous speed (FIG. 45), lower arrest coefficient (FIG. 46), higher mean displacement speed (FIG. 47), higher meandering index (FIG. 48) and higher linearity of forward progression (FIG. 49). *p<0.05 and **p<0.01.

FIGS. 50 and 51 are charts illustrating individual putative T cell tracks prior to CL wear (pre-CL) (FIG. 50) and after 3 hours of CL wear (post-CL) (FIG. 51), after normalization of starting positions to the origin with *p<0.05 and **p<0.01. These plots show greater cell mobility in the post-CL condition relative to the pre-CL condition.

FIGS. 52-58 relate to Example 1C where putative dendritic cells (DCs) use their dendrites to dynamically survey the human corneal epithelium and show muted responses when exposed to inflammatory stimuli.

FIG. 52 is a chart illustrating that DC densities were similar across participant groups (healthy, n=16; untreated allergy, n=10; treated allergy, n=4) in the central cornea (p>0.05). In individuals with untreated allergy, there were fewer DCs in the paracentral (whorl) corneal region relative to healthy controls (*p<0.05).

FIG. 53 is a chart illustrating that DCs with more dendritic complexity (tips/cell) generally had a higher mean dSEARCH index per minute (R²=0.41, p=0.0022).

FIG. 54 is a chart illustrating that the mean normalized dSEARCH was lower in cells in individuals with untreated allergy (n=2 cells), relative to healthy controls (n=18 cells).

FIG. 55 is a chart illustrating that cell densities were unchanged after three hours of CL wear.

FIG. 56 illustrates sequential time-lapsed images acquired over 30 minutes, with two individual cells noted (labelled 1 and 2), at the pre-CL (upper row) and post-CL (lower row) timepoints. Cell (1) undergoes a shift in its dendritic orientation post-CL wear, whereas the overall dendritic conformation of Cell (2) is unchanged. Scale bar applies to all images.

FIG. 57 illustrates colour time-lapsed merges of the upper and lower panels shown in FIG. 56, respectively. The Pre-CL merge shows more obvious dSEARCH activity (Cell 1>2), which is reduced in the post-CL merge. Scale bar applies to both images.

FIG. 58 is a chart illustrating data for matched cells (n=32), analysed pre-CL vs post-CL wear, showing that the normalized average dSEARCH index was lower post-CL wear with *p<0.05.

FIGS. 59-65 relate to Example 1D where the new IVCM technique captures dynamic immune cell-to-cell interactions and neuro-immune interactions in the human cornea.

FIG. 59 illustrates sequential time-lapsed images of the corneal epithelium, acquired over 35 minutes, showing examples of a putative T cell crossing a sensory nerve axon (green triangle) and a separate putative T cell ‘kissing’ a nerve before rebounding back to its original location (blue arrow). A colour time-lapsed merge highlights the cell motility dynamics. Scale bar applies to all images.

FIG. 60 is a chart illustrating for matched cells seen to ‘kiss’ nerves (n=5), mean instantaneous speed is higher after cells interacted with a nerve (‘After’) relative to approaching the nerve (‘Before’).

FIG. 61 illustrates sequential time-lapsed images, and a corresponding colour time-lapsed merge in FIG. 62, showing dynamic interactions between a putative T cell, putative DC and nerve axon.

FIG. 63 illustrates sequential time-lapsed images showing a putative DC dendrite dynamically moving across a nerve axon (green arrow). Scale bar applies to all images.

FIG. 64 illustrates a magnified inset (orange box) of the final image in panel of FIG. 63, to highlight an interaction between a putative DC and a putative T cell in the human cornea using IVCM.

FIG. 65 illustrates wholemount immunofluorescent images from a mouse corneal epithelium, stained for cell nuclei (Hoechst), DCs (CD11c⁺) and T cells (CD3⁺); the DC-T cell interaction mirrors the appearance of the in vivo human corneal imaging shown in FIG. 64.

FIG. 66-78 relate to Example 1E where putative stromal macrophages are morphologically and behaviorally distinct from resident tissue keratocytes and are affected by allergy and acute stimulation elicited by CL wear.

FIG. 66 illustrates sequential time-lapsed images of the anterior stroma in a healthy control cornea over a 20-minute period. Stationary keratocytes (pink arrows) and motile cells (orange arrowheads) demonstrate differential behaviours. A colour-coded hyper stack shows cytoplasmic displacement in macrophages, and cumulative cell masks show small shaped keratocytes (magenta) compared to ameboid shaped macrophages (cyan).

FIG. 67 is a chart illustrating that keratocytes represent the major cell type in the anterior corneal stroma, followed by putative macrophages and T cells.

FIG. 68 is a chart illustrating that the coefficient of variation of cell shape over time was higher in putative macrophages compared to keratocytes.

FIG. 69 is a chart illustrating that there were fewer stromal macrophages in individuals with untreated and treated allergy compared to healthy controls. Per cell analysis charts of cell area (FIG. 70), perimeter (FIG. 71) and circularity (FIG. 72) showed differences in the morphology of stromal macrophages in controls versus both treated and untreated allergy. *p<0.05, **p<0.01, ***p<0.001, and ****p<0.0001.

FIG. 73 illustrates representative time-lapsed colour coded hyperstacks of corneal stromal macrophages in healthy control, untreated allergy and treated allergy individuals.

FIG. 74 illustrates time-lapsed image sequence and corresponding colour-coded hyperstacks and cumulative cell area masks of a stromal macrophage pre-CL (top row) and post-CL (bottom row).

FIG. 75-78 are charts illustrating repeated measures comparisons of the cell area (FIG. 75), perimeter (FIG. 76), solidity (FIG. 77) and circularity (FIG. 78) of the same stromal macrophages (n=9), which were quantified pre-CL and post-CL. Scale bars are as indicated.

FIGS. 79-82 relate to Example 2 where the novel imaging technique was used to track the effect of environmental seasonal-driven factors on the density, morphology and dynamic behaviour of human corneal immune subsets.

FIG. 79 provides a summary of the design of Example 2 and the in vivo confocal microscopy imaging (IVCM) protocol. (A) Healthy adult participants aged between 20 and 40 years attended two visits. (B) Time-lapsed corneal imaging was performed in the corneal whorl region, and static IVCM images were collected from both the whorl and central cornea.

FIG. 80 illustrates the season-dependent changes to morphodynamics of corneal intraepithelial dendritic cells (DCs). The morphology and behaviour of DCs were significantly affected by season, with cells generally having a larger, more elongated and more concave shape, and a greater dendritic complexity, but a lower dendrite tip probing speed, during the Visit 1 (Spring/Summer) relative to Visit 2 (Autumn/Winter). (A) Representative corneal in vivo confocal microscopy images showing DCs in the basal epithelium, with their field area (yellow) and cell area (red) delineated, in a representative participant at Visits 1 and 2. (B) DC density was similar at each study visit. (C-H) Effect of season on various morphological features of DCs. At Visit 1, DCs had more tips (C), a greater field area (D), no change in cell area I, larger cell perimeter (F), lower circularity (G) and lower solidity (H). (I) Representative time-lapsed images showing DC probing behaviour. (J-K) Effect of season on the dynamic behaviour of DCs. At Visit 1, DCs had a lower probing speed (J) but similar dSEARCH index (K) compared to Visit 2. **p<0.01, ***p<0.001. Data are shown at the ‘per participant’ level for dendritic cell density (Panel B), and ‘per cell’ level for morphology and dynamic parameters (Panels C-H, J-K), with the latter involving a mixed effects model for the statistical analysis.

FIG. 81 highlights that the morphology and dynamics of corneal intraepithelial T cells are unaffected across seasons. (A) Representative IVCM images showing T cells (arrows) at the level of corneal sub-basal nerve plexus. (B) Plot showing similar T cell densities in the central and whorl regions, at both study visits. There was an absence of season-dependent differences for T cell morphology, for each of cell area (C), perimeter (D), circularity (E) and solidity (F). T cell dynamic behaviours were also unaffected, with no differences in instantaneous speed (G), displacement speed (H) or meandering index (1) across seasons. (J) Representative time-lapsed IVCM images showing the patrolling behaviour of a T cell; the final panel shows the movement trajectory of the cell. Data are shown at the ‘per participant’ level for T cell density (Panel B), and ‘per cell’ level for morphology and dynamic parameters (Panels C-I), with the latter involving a mixed effects model for the statistical analysis.

FIG. 82 illustrates season-dependent changes to the morphodynamics of corneal stromal macrophages. The morphology and behaviour of stromal macrophages were affected by season, with cells having a more circular and convex shape, and lower dynamic activity, during Visit 1 (Spring/Summer) relative to Visit 2 (Autumn/Winter). (A) Representative IVCM images showing macrophages in the superficial corneal stroma (yellow arrows and red cell outlines). These macrophages are challenging to differentiate from static keratocytes (white cells without indicators) from static IVCM images but are readily distinguishable in the time-lapsed videos based on their movement. (B) Corneal stromal macrophage density was unchanged across visits. There were no differences for cell area (C) or perimeter (D) across seasons, but cell circularity (E) and solidity (F) were higher at Visit 1 relative to Visit 2. For dynamic cell behaviours, there was no inter-season difference for instantaneous speed (G) or displacement speed (H), but cells showed a lower ‘dancing index’ at Visit 1 relative to Visit 2 (1). (J) Representative images showing a stromal macrophage changing its morphology over the imaging period of a time-lapsed video. (K) Representative images showing how the ‘dancing index’ is calculated for a single cell, to quantify the change in macrophage shape over time. **p<0.01, ***p<0.001. Data are shown at the ‘per participant’ level for stromal macrophage density (Panel B), and ‘per cell’ level for morphology and dynamic parameters (Panels C-I), with the latter involving a mixed effects model for the statistical analysis.

FIGS. 83-85 relate to Example 3 which demonstrates evidence for the ability to use the imaging method to capture temporal (diurnal) changes in immune cell behaviours.

FIG. 83 illustrates that, quantified using in vivo time-lapsed imaging to track individual immune cells, T cells in the cornea were more motile in the morning [AM], shown by greater cell colouration in the time-lapsed image merge, relative to evening [PM]. Dendritic cells (DCs) had the opposite change with lower cell ‘probing’. Chemoattractant (CCL2) concentrations in the tears also showed diurnal differences.

FIG. 84 shows corneal T cell instantaneous speed, displacement speed, meandering index and mobility in the morning [AM] relative to afternoon/evening [PM].

FIG. 85 shows Corneal dendritic cell (DC) dynamics, quantified using the dSEARCH index and probing speed, in the morning [AM] relative to afternoon/evening [PM].

FIGS. 86 and 87 relate to Example 4 which provides evidence for the ability to use the new imaging technique to quantify changes to corneal immune cells in a systemic disease (HIV).

FIG. 86 provides a comparative analysis of corneal T cell dynamic parameters in the peripheral and whorl regions. Each data-point represents one cell. In HIV+ individuals, T cells show a higher instantaneous speed than T cells in HIV− (control individuals) in the peripheral corneal region.

FIG. 87 provides a comparative analysis of the percentage (proportion) of T cells dichotomously classed as mobile (speed >2 μm/min in at least one time interval during the imaging period) vs immobile (speed <2 μm/min over the entire imaging period). All values are presented as mean plus/minus standard deviation (SD).

FIGS. 88 and 89 relate to Example 5 which shows the ability to use the imaging method to quantify changes to corneal immune cell dynamics in response to a topical pro-inflammatory stimulus (hyperosmotic saline).

FIG. 88 illustrates a comparative analysis of the instantaneous speed of corneal T cells assessed before and after topical stimuli of different osmolarity (‘iso-osmolar’: 300 mOsm/L and ‘hyper-osmolar’: 1500 mOsm/L). T cell instantaneous was significantly higher after exposure to hyper-osmolar saline eye drops, relative to the iso-osmolar condition.

FIG. 89 illustrates a comparative analysis of the dSEARCH probing behaviour (upper row) and dendrite probing speed (lower row) of the same corneal epithelial dendritic cells (DCs), assessed both before and after topical stimuli of different osmolarity (‘iso-osmolar’: 300 mOsm/L and ‘hyper-osmolar’: 1500 mOsm/L). DC dynamic behaviours were unaffected by either stimulus.

FIG. 90 to 101 describe a system and method for image processing which improves the efficiency and consistency of time-lapsed video creation from static images taken from a scanning instrument, to enable the collection of dynamic data for clinical and research applications.

FIG. 90 is an image processing method for a scanning instrument for scanning biological tissue, relating to receiving two or more images of biological tissue, selecting a first reference image, translating the two or more images to align with the first reference image, determining a degree of intra-image warp and automatically selecting a second reference image based on the degree of intra-image warp, and then averaging and registering the two or more images.

FIG. 91 illustrates flat-fielding of the acquired video image frames. (A) An example raw image frame, acquired at the level of the human corneal basal epithelium using IVCM. (B) Flat-field image computed as the average of 100 unregistered video frames. (C) Results of flat-fielding operation applied to the frame shown in A. Scale bar=60.

FIG. 92 illustrates differing similarity to the reference image frame used for full-field translation. (A) Reference image frame selected. (B) Example image frame with high similarity to the reference. A horizontal arrow identifies the same immune cell in each frame. (C) Example image frame with low similarity to the reference; the eye position has shifted substantially compared with the other frames such that the imaged patch of tissue is less similar, as evident by the new location of the indicated cell. Scale bar=60.

FIG. 93 shows pixel count histograms for the image frames shown in FIG. 92. The blue and red plots correspond to FIGS. 92(A) and 92(B) respectively, showing high similarity. The yellow plot shows a slightly different histogram shape and includes less overlapping tissue, as was shown in FIG. 92(C).

FIG. 94 demonstrates the outcome of full-field translations. (A) The average intensity for full-field translations within a single 100 frame video. Scale bar=60 μm. (B) Change in eye position over time implied by the full-field translations. Missing data indicates registrations that were rejected.

FIG. 95 illustrates the first stage of strip registration. (A) Reference image frame. Coloured boxes (9510, 9515, 9520, and 9525) indicate the progressive reduction in strip height across three iterations and have been shifted to show the outcome of strip registration (i.e., to align with the corresponding tissue in B. Scale bar=60 μm). (B) Representative image frame to be registered. (C) Example image frame after strip registration; the position of the reference tissue image is shown by the green border (9540). (D) Total intra-frame strip shifts required and used to produce panel C. (E) Incremental strip shifts required on the third iteration of strip registration (corresponding to the thinnest, 9520 in A and B). (F) Averaged image showing the outcome of strip registration.

FIG. 96 illustrates the second stage of strip registration. (A) A heuristic to select the least distorted video image frame; the frame chosen is shown by a green dashed line (9620). The frames chosen for the first round of strip registration (black dashed line, 9625) and for full-field translation (red dashed line, 9615) were more distorted, according to this heuristic. (B) The reference frame selected (corresponding to 9620 in A). Scale bar=60 μm. (C) The outcome of strip registration, shown by the average registered pixel intensity. The green border indicates the location of the reference tissue.

FIG. 97 illustrates the third (final) stage of strip registration. (A) Estimated eye position across the full video image acquisition, with the reference frame period shown by the dashed lines (9725). (B) Full-field translations for the same reference image frame, shown for comparison. (C) Final strip-registered average image, excluding pixels illuminated less than three times. This image was used to create time-lapsed videos in Example 6. Scale bar=60 μm.

FIG. 98 demonstrates the registration of averaged images collected at different time points, to create a time-lapsed video. (A) Chosen reference time point. Scale bar=100 μm. (B) Data from another time point, four minutes after timepoint in A, after translation and rotation of the image. (C) ‘Vesselness’ filter applied to the image shown in panel B, to minimize the influence of the empty background tissue. (D) Horizontal deformation map, required along with E to warp the image so that it matches the reference. (E) Vertical displacement map. (F) Registered image, being a warped version of panel B.

FIG. 99 provides representative images from a time-lapsed video acquired from the basal epithelium of the peripheral corneal region. (A) A colour-merged, time-lapsed image shows the dynamics of human corneal epithelial immune cells, whereby coloured regions (9905 and 9910) indicate cell movement. (B1-B6) Motile T cells show patrolling behaviours across the 30-min imaging period. (C1-C6) A dendritic cell shows no obvious displacement of its soma, but instead extends and retracts its dendrites. Scale bar=50 μm.

FIG. 100 provides representative images from a time-lapsed video acquired from the anterior stroma of the peripheral corneal region. (A) A colour-merged, time-lapsed image shows the dynamic of human corneal stromal macrophages. (B1-B6) (C1-C6) Stromal macrophages show alterations to their morphology across the 30-min imaging period. Scale bar=50 μm.

FIG. 101 illustrates the applicability of the automated protocol to confocal retinal data collected with an adaptive optics scanning laser ophthalmoscopy (AOSLO) system. (A) A single frame after registration within a video, demonstrating successful handling of a large microsaccade. (B) Average image from 100 registered video frames. (C) Average of 15 time-lapsed average images. Data acquired at 790 nm, with fixation at 0.5° from the foveal centre (visible bottom left) and shown on log scale to improve visualization of cells. Scale bar=100 μm.

### DETAILED DESCRIPTION

Before describing the present disclosure in detail, it is to be understood that the terminology used herein is for the purpose of describing particular embodiments only and is not intended to be limiting.

The present invention may be utilised in the context of corneal imaging, and it will be convenient to describe the invention in relation to that exemplary, but non-limiting, application. It will be appreciated that the present invention is not limited to that application and may, for example, be applied in another context such as imaging of other regions of the eye, for example, the retina, or other biological tissues.

As used in this specification and the appended claims, the singular forms “a”, “an” and “the” include plural referents unless the content clearly dictates otherwise.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art.

All publications or patent applications or patents cited herein are entirely incorporated herein by reference.

A reference herein to a publication or patent application or patent or other matter which is given as prior art is not to be taken as admission that the document or matter (e.g., databases) was known or that the information it contains was part of the common general knowledge as at the priority date of any of the claims.

Embodiments of the disclosure are discussed herein by reference to the drawings which are not to scale and are intended merely to assist with explanation of the disclosure.

As used herein the term “subject” is intended to include a mammal, a human or nonhuman primate, a dog, a cat, a horse, a cow, other farm animals, or a rodent (e.g., mice, rats, guinea pig. etc.). The term “subject,” “patient,” and “individual” are used interchangeably herein. The term “patient” as used herein includes a living human or non-human subject that is receiving medical care or that should receive medical care due to a disease or condition. This includes subjects with no defined illness or observable symptoms of a disease or condition who are being investigated for signs of pathology. Thus, the methods described herein are applicable to both, human and veterinary disease.

In some examples, the term “subject” refers to subjects at risk of or with symptoms of chronic or acute inflammation of the eye.

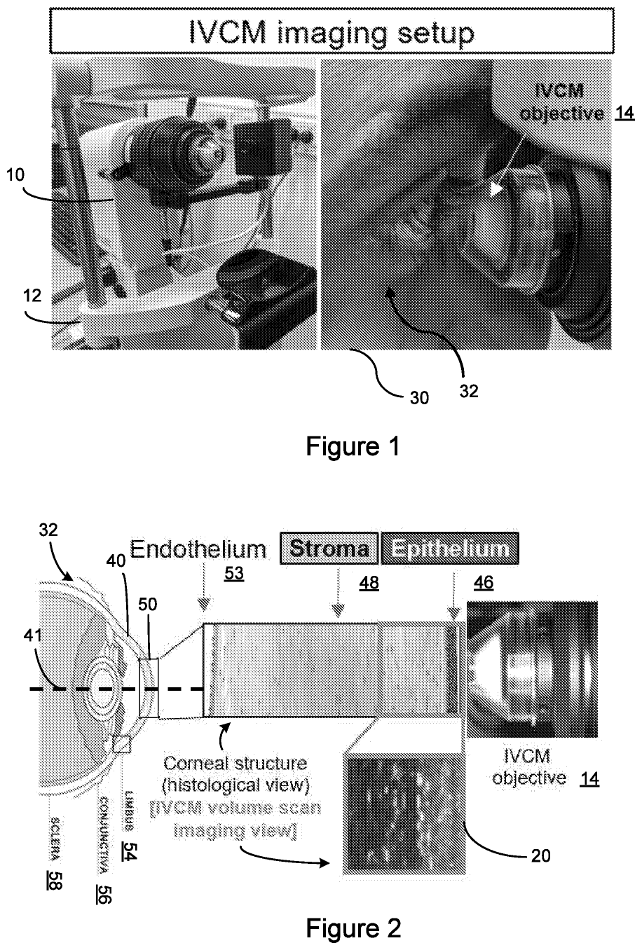

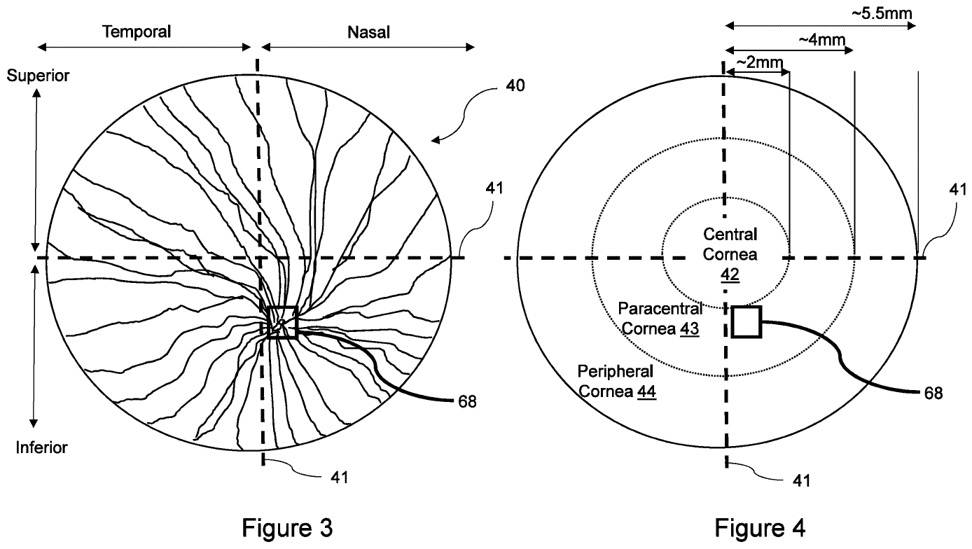

Reference herein to “superior” means above. Reference herein to “inferior” means below. In non-limiting embodiments of the disclosure, the subject adopts an upright position for acquiring the microscopy images of the biological tissue, such as corneal tissue of the subject's eye (see e.g., FIG. 1). In this context, the term “superior” is used herein to reference features or elements that are above a central or midline axis of the eye and “inferior” is used herein to reference features or elements that are below the central axis or midline of the eye (see e.g., FIGS. 2 and 3).

As used herein the term “location”, e.g., first location, second location or subsequent location, is used to define a general region or area in the focal plane (x, y) of the biological tissue for imaging. For example, the term “location” may reference a region of the biological tissue in the focal plane (x, y) such as the central corneal region of corneal tissue (see e.g., FIG. 4). The term “position” is used herein to define a specific location in the focal plane (x, y) which may include a number of different positions within the same region of the biological tissue for imaging (particularly relevant for sequence scanning). For example, the term “position” may reference different positions for acquiring microscopy images within the central corneal region of corneal tissue (see e.g., FIG. 4).

Embodiments of the disclosure are discussed herein by reference to in vivo imaging of a biological tissue of a subject over time. However, the embodiments are not limited to in vivo imaging of live tissue, that is imaging of tissue within or part of a living subject (i.e., a living tissue). The imaging methods disclosed herein may be applicable for ex-vivo imaging of a tissue, such as donor tissue, or tissue sample; or in vitro imaging of an engineered tissue such as scaffold based tissue or cell sheet.

The imaging methods disclosed herein are useful for imaging biological tissue from an eye of a subject, whether it be a human or animal subject, in various embodiments of the disclosure. The biological tissue may be ocular surface tissue. The biological tissue may be corneal tissue. The corneal tissue may be imaged at one or more locations in a focal plane of a corneal tissue layer comprising the epithelium, stroma or endothelium. The biological tissue may be retinal tissue. The retinal tissue may be imaged at one or more locations in a focal plane of a retinal tissue layer comprising, for example, the Retinal Pigment Epithelium (RPE), Photoreceptor Layer, Outer Nuclear Layer (ONL), Outer Plexiform Layer (OPL), Inner Nuclear Layer (INL), Inner Plexiform Layer (IPL), Ganglion Cell Layer (GCL), Nerve Fiber Layer (NFL), and Internal Limiting Membrane (ILM).

The imaging methods may be particularly useful for imaging cells in the epithelium and stroma of the cornea. However, the embodiments are not limited to this application and the imaging methods disclosed herein may be applicable for imaging static or dynamic objects other than cells in corneal tissue or in other tissues or regions of the subject. The types of tissue imaged may include connective tissue, epithelial tissue, muscle tissue, and/or nervous tissue. The tissue may be located in various regions, organs or systems of the subject's body, such as but not limited to non-invasive regions or access sites including the eye, nose, mouth or ear. Desirably, the tissue imaged may include one or more immune cells for analysis of cell behaviour.

Embodiments of the disclosure discussed herein are useful for determining the presence of at least one object in one or more of the microscopy images acquired over time. The at least one object may be a static or dynamic object over the time period. The dynamic object may be moving or motile. The static object may not move or be motile. The static object may be a non-immune cell. The non-immune cell may be a fibroblast, for example. In particular, the non-immune cell may be a corneal keratocyte in corneal tissue. The object may be an intrinsic or extrinsic feature of the biological tissue. The intrinsic feature may comprise one or more of a nerve or part thereof (e.g., an axon), a pattern of nerves, a cell or part thereof, a pattern of cells and at least one feature of connective tissue. The extrinsic feature may comprise one or more of a cell, a pattern of cells, and an organism (either living or non-living), or a foreign-body entity, to name a few. The organism may comprise one of an amoeba, a bacteria or a fungi. In particular, the organism may comprise Acanthamoeba cysts, bacteria or fungal filaments, which are found e.g., in corneal infection.

The imaging methods may be particularly useful for determining the presence of at least one cell in a biological tissue of the subject's body, such as the cornea of the subject's eye. The at least one cell may be an immune cell. The immune cells may be identified based on the tissue depth and/or location in the focal plane (x, y). The immune cells may be identified as one of a putative dendritic cell, a putative T cell and a putative macrophage, to name a few. However, the embodiments are not limited to imaging of immune cells or the cell types identified, and may be useful for imaging of other cell types such as mast cells, natural killer cells, intraepithelial lymphocytes, B cells, neutrophils, natural killer T cells, eosinophils, basophils, platelets, and plasma cells.

The presence of an object, e.g., a cell, may be determined based on evaluations of object shape (e.g., cell shape) in the series of microscopy images over the time period. An object may be identified based on one or more morphological parameters according to some embodiments of the disclosure. The evaluations of object shape and the one or more morphological parameters may be performed by processing of the microscopy images using one or more imaging processing techniques as will be described herein. An example of such imaging processing techniques is set forth in the Materials and Method section of the Examples. A person skilled in the art would appreciate that embodiments of the disclosure are not limited to the specific image processing techniques described in the Examples and that the skilled person could implement various techniques known in the art to provide the evaluations of object shape (e.g., cell shape) based on the one or more morphological parameters.

An example of various morphological parameters for determining the presence of cells are shown in Tables 1 and 2 of the Examples and also described in the Materials and Methods section of the Examples. The one or more morphological parameters may be selected from at least one of a group comprising: field area, cell area, perimeter, circularity, roundness and solidity. Preferably for identifying immune cells, the one or more morphological parameters include at least field area, cell area and solidity. The field area may be measured by the total area encompassed by a polygon approximation defined by the outermost aspects of the cell and is measured in micrometres squared (μm²). The cell area (μm²) may be measured by the total area encompassed by the cell border which may be determined using a manual freehand trace around the cell border. The perimeter (μm) is the distance around the edges of the cell which may be determined by tracing around the cell border. The circularity is calculated by 4π×[field area/perimeter²]. The roundness is calculated by 4×field area/[π×major_axis²]. The solidity is calculated by the cell area divided by the convex hull area. The morphological parameters may also include cell density (cells/mm²) which is the number of cells per area of the region of tissue. For identification of dendritic cells (DCs), the morphological parameters may also include dendritic complexity (number of dendritic tips per cell). Although these parameters have been described with reference to a cell, it is to be understood that the morphological parameters could equally apply to other objects of interest, such as a nerve, a connective tissue feature, an organism (either living or non-living), or a foreign-body entity, to name a few.

The values for the one or more morphological parameters in the examples (e.g., Table 2) have been quantified based on studies of immune cell subsets in healthy human corneas using the new IVCM imaging technique disclosed herein (excluding cell density and dendritic complexity). Studies by the inventors have shown that the epithelium of the cornea may include putative T cells and putative dendritic cells (DCs) and the stroma of the cornea may include putative macrophages and putative T cells (see Table 2 and Examples). The experimental values for the parameters in Table 2 may vary approximately or about the ±standard deviation (SD) stated and such acceptable variation in the ranges of values for identifying the cell types would be appreciated by a person skilled in the art. The definition and calculation or morphological and dynamic corneal immune cell parameters are described in Table 1.

In the epithelium of corneal tissue, putative T cells may be identified based on the following parameters: field area (μm²) in a range of about 30 μm² to about 130 μm²; cell area (μm²) in a range of about 30 μm² to about 80 μm²; perimeter (μm) in a range of about 25 μm to about 60 μm; circularity in a range of about 0.3 to about 0.6; roundness in a range of about 0.3 to about 0.6; and solidity in a range of about 0.6 to about 0.9.

In the epithelium of corneal tissue, putative dendritic cells may be identified based on the following parameters: field area (μm²) in a range of about 850 μm² to about 1500 μm²; cell area (μm²) in a range of about 140 μm² to about 260 μm²; perimeter (μm) in a range of about 130 μm to about 310 μm; circularity in a range of about 0.02 to about 0.11, for example, 0.02 to about 0.06; roundness in a range of about 0.25 to about 0.75; and solidity in a range of about 0.1 to about 0.3.

In the stroma of corneal tissue, putative macrophage cells may be identified based on the following parameters: field area (μm²) in a range of about 275 μm² to about 680 μm²; cell area (μm²) in a range of about 215 μm² to about 420 μm², for example, about 215 μm² to about 345 μm²; perimeter (μm) in a range of about 75 μm to about 125 μm; circularity in a range of about 0.25 to about 0.55; roundness in a range of about 0.25 to about 0.65; and solidity in a range of about 0.5 to about 0.8.

In the stroma of corneal tissue, putative T cells may be identified based on the following parameters: field area (μm²) in a range of about 100 μm² to about 270 μm²; cell area (μm²) in a range of about 100 μm² to about 120 μm²; perimeter (μm) in a range of about 45 am to about 65 μm; circularity in a range of about 0.35 to about 0.6; roundness in a range of about 0.25 to about 0.65; and solidity in a range of about 0.3 to about 0.8.

Embodiments of the disclosure discussed herein relate to imaging methods for monitoring dynamic behaviour of at least one object (e.g., a cell) in microscopy images acquired over a time period. Dynamic behaviour may include temporal and/or spatial variation of the object(s) (e.g., cell(s)), in potentially two or three dimensions, in the acquired microscopy images which are monitored over the time period. Where both variations are included, the dynamic behaviour monitored may be spatio-temporal behaviour. The imaging methods may enable monitoring of object behaviour based on one or more dynamic parameters of the object(s) in the microscopy images. This may include tracking movement (e.g., dynamic motion) of the object across a range of focal depths (z) of the biological tissue over the time period. This may also include tracking cellular motility.

The evaluations of dynamic behaviour may be performed by processing of the microscopy images using one or more imaging processing techniques as will be described herein. An example of such imaging processing techniques is set forth in the Materials and Method section of the Examples. A person skilled in the art would appreciate that embodiments of the disclosure are not limited to the specific image processing techniques described in the Examples and that the skilled person could implement various known techniques known in the art to provide the evaluations of dynamic behaviour.

An example of dynamic parameters for monitoring cell behaviour is shown in Table 1 in the Examples. The one or more dynamic parameters may be selected from at least one of a group comprising: cell instantaneous speed, cell displacement speed, meandering index, displacement outreach ratio, lower arrest coefficient, linearity of forward progression, mean directional change, dendrite surveillance extension and retraction cycling habitude (dSEARCH), probing speed, outreach ratio, and dancing index. The cell instantaneous speed may be measured in micrometres per minute (μm/min). The cell displacement speed may be the net cell displacement divided by the total time. The meandering index may be the net cell displacement divided by the total track length. The displacement outreach ratio may be the maximum cell displacement divided by the total track length. The lower arrest coefficient may be the percentage of time that the cell speed is less than 2 μm/min. The linearity of forward progression may be the ratio of the displacement speed and the mean instantaneous speed. The dSEARCH parameter is a parameter known in the art for measuring dSEARCH behaviours of dendritic cells including extension and retraction of the dendrites. The dSEARCH parameter may be quantified per minute (normalized dSEARCH index) which represents the cumulative distances of dendrite extension and retraction observed for a given dendritic cell per unit of time. Although these parameters have been described with reference to a cell, it is to be understood that the dynamic parameters could equally apply to other objects of interest, such as a nerve, a connective tissue feature, an organism (either living or non-living), or a foreign-body entity, to name a few.

### Novel IVCM Imaging Technique

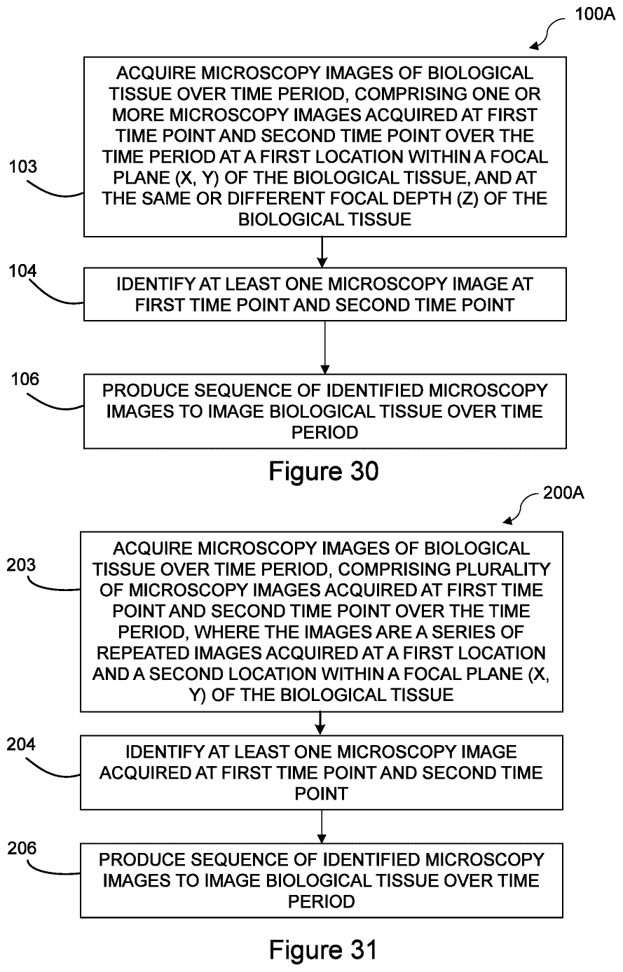

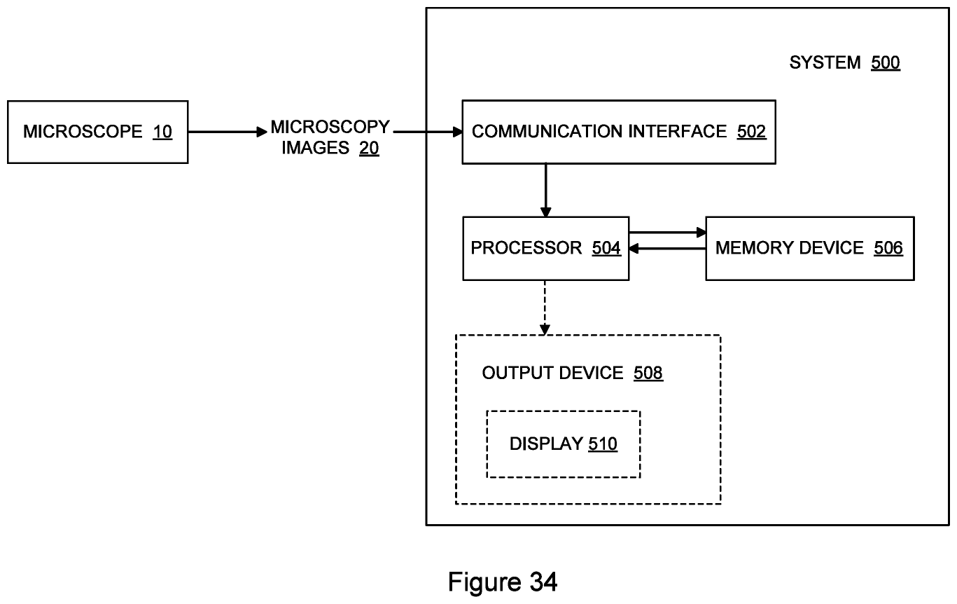

In some embodiments, the inventive methods disclosed herein comprise the step of providing microscopy images of the biological tissue over a time period. The step of providing microscopy images may comprise acquiring the microscopy images of the biological tissue over the time period (e.g., see step 103 and 203 of FIGS. 30 and 31). That is, the providing step may optionally require an operator or programmed system (e.g., see system 500 of FIG. 34) to acquire the microscopy images for use in the inventive methods. The step of acquiring the microscopy images may comprise the step of operating an imaging system configured to acquire enface microscopy images of the biological tissue over the time period. The imaging system may include a microscope 10 or may be a microscopy imaging system.

Turning to FIG. 1, there is illustrated a microscope 10 for acquiring microscopy images of biological tissue 40 over time, for example, corneal tissue of a subject's eye 32. The microscope 10 includes a head rest 12 for receiving the chin and forehead of the subject 30. In preferred embodiments for imaging of the human cornea 40, the subject 30 is positioned with their chin and forehead resting on the head rest 12 and their eye 32 is positioned in line with the objective 14 of the microscope 10. The microscope 10 is preferably an in vivo confocal microscope (IVCM), and more preferably a laser-scanning in vivo confocal microscope (IVCM). IVCM systems have the advantage of being able to image through moderately opaque tissues and also being able to observe dynamic processes in the tissue, particularly suitable for the cornea 40 of a subject's eye 32. However, embodiments of the disclosure are not limited to use of in vivo confocal microscopy (IVCM) systems. The microscope 10 may be any microscope suitable for en face imaging of a biological tissue 40 of a subject 30 over time. The microscope or microscopy system may not involve confocal microscopy and may instead be an optical coherence microscopy (OCM) system. The OCM system is an alternative imaging approach to live fluorescence microscopy which uses tomography, i.e., optical coherence tomography (OCT), in a microscopy system.

In some embodiments, the microscope 10 shown in FIG. 1 is a laser-scanning, in vivo confocal microscopy (IVCM) system with the Heidelberg HRT-3 with Rostock Corneal Module, to acquire images of the human cornea. The Rostock Corneal Module is a contact system attached to the HRT-3 and features three unique imaging modes including (i) section scan to acquire a single image, (ii) volume scan to acquire multiple images across a selected depth of the region of interest (e.g., collecting 40 images over a depth of approximately 80-100 μm), and (iii) a sequence scan to acquire multiple frames in a fixed focal plane and depth over a short time period (e.g., collecting 100 image frames over several seconds).

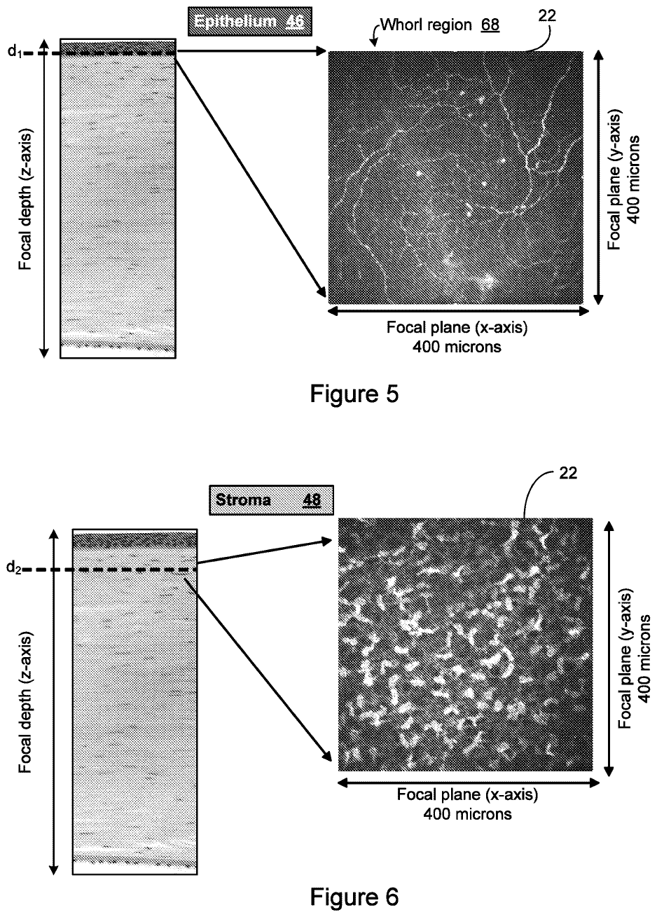

The images acquired are en-face, that is parallel to the surface of the cornea 40 as best shown in FIGS. 3 and 4. The operator can manually select the depth of interest and adjust the image brightness. The images may have a field of view of either 300×300 μm or 400×400 um (depending on the device lens power) with the lateral resolution of approximately 1-2 μm and the axial resolution of approximately 4 μm. Images acquired and used in embodiments of the disclosure may include a field view of 400×400 μm as shown in FIGS. 5 and 6. In alternative embodiments, images may be acquired using a slit-scanning microscope instead, such as Confoscan Series IVCM system (Nidek Co. Ltd, Gamagori, Japan Confoscan Series, have the following parameters: approximately 1-2 μm for lateral resolution and approximately 25-27 μm for axial resolution.

FIG. 2 is a schematic diagram illustrating the laser-scanning IVCM system of FIG. 1 shown diagrammatically in histological cross-section (not to scale) and showing a volume scan across a predetermined range of focal depths (z) at a first location 50 of the cornea 40 of the subject 30. The subject's eye 32 is depicted including the cornea 40, limbus 54, conjunctiva 56 and sclera 58. In this case, the microscope 10 is an IVCM system with objective 14 and a volume scan with one or more microscopy images 20 is able to be acquired. FIG. 2 illustrates a histological view of the corneal structure across the depth of the cornea 40 that is imaged. A snap-shot highlighted with a square shows microscopy images 20 obtained using the microscope 10 for the volume scan. Various tissue layers of the cornea 40 are able to be imaged including the epithelium 46, the stroma 48 and the endothelium 53.

FIG. 3 is a cross-sectional diagram through corneal tissue 40 of a subject 30 along the broken line 41 in FIG. 2 representing the central axis or midline of a subject's eye 32. FIGS. 3 and 4 illustrate 2D en face regions in a focal plane (x, y) showing the nerve pattern representing the whorl region (FIG. 3) and a location 68 for imaging the whorl in the inferior paracentral cornea 23 (FIG. 4). FIGS. 3 and 4 also provide an indication of the direction of imaging being either superior (above) or inferior (below) relative to the central axis 41 with the subject 30 being in an upright position, and being either temporal (towards the brain) or nasal (towards the nose) relative to the central axis 41.

FIG. 4 is a schematic diagram showing regions of the cornea 40 comprising the central cornea 22, the paracentral cornea 23 and the peripheral cornea 24, where the whorl region 68 is located in the inferior paracentral corneal region 23. The relative radii of the regions of the cornea 40 is also illustrated, where the radius of the central cornea 22 is about 2 mm, the radius of the paracentral cornea 23 is about 4 mm and the radius of the peripheral cornea 24 is about 5.5 mm. The relative radii are based on average measurements of a normative subject's eye 32 which may vary based on e.g., the subject's size, gender and anatomy.

FIGS. 5 and 6 illustrate a single en face microscopy image 22 identified from the volume scan of FIG. 2 at the depth d₁ of the epithelium 46 (FIG. 5) and at the depth d₂ of the stroma 48 (FIG. 6). Each of the images 22 are 400 μm×400 μm in the focal plane (x-y axis) (parallel to the surface of the cornea 40) and extracted at a particular depth in the z-plane (z-axis). FIG. 5 illustrates a pattern of nerves called the “whorl” region 68 which is located in the epithelium 46 of the cornea 40. The whorl region 68 is an anatomical landmark which is often used to identify a location or region 50 within the corneal tissue for imaging, and to also find the same location or region for subsequent imaging. FIG. 6 illustrates a number of immune cells in the identified image 22 in the stromal layer 48 of the cornea 40.

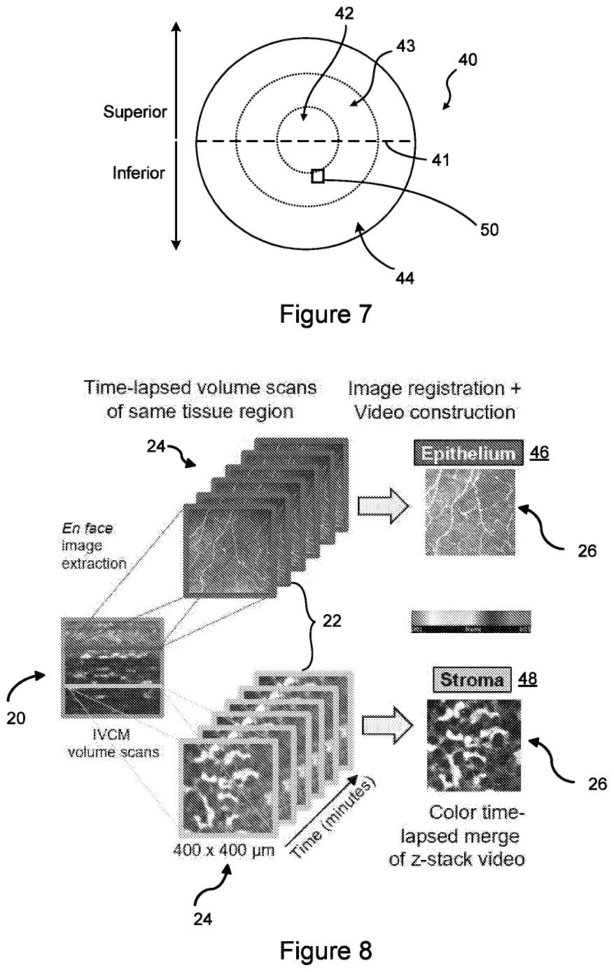

FIG. 7 is a schematic diagram of a cross-section of a cornea 40 of the subject 30 showing a first location 50 indicated as the region of tissue for volume imaging, according to some embodiments of the disclosure. The first location 50 is within the paracentral cornea 43, which is surrounding by the peripheral cornea 44. The first location 50 to be imaged is more specifically located in the inferior paracentral cornea 43. Each image is a two-dimensional (2D) image acquired enface in the focal plane (x-y) which is illustrated in FIGS. 5 and 6. A plurality of microscopy images 20 for the volume scan are then acquired across a predetermined range of focal depths (z), being in the z-plane (z-axis) that is perpendicular to the page through the cross-sectional diagram of FIG. 7. The predetermined range of focal depths (z) may be in a range of about 50 μm to about 100 μm (for example, about 60 μm to about 80 μm, or about 80 μm to about 100 μm), e.g., for imaging of the corneal stromal layer. The predetermined range of focal depths (z) are not limited to this range and may be more than 0 μm to about 100 μm. The plurality of microscopy images 20 acquired may comprise about 2 images to about 40 images for the volume scan.

FIG. 8 is a schematic diagram illustrating volume imaging of the cornea 40 of the subject 30 at two depths, namely at the level of the epithelium 46 and the stroma 48, and at the same location or region of tissue 50, and creation of time-lapsed volume scans 24 of the same location or tissue region 50 and colour time-lapsed merges of the images, according to some embodiments of the disclosure. The novel IVCM method involves in some embodiments sequentially capturing a plurality of microscopy images 20 which may be a high-resolution volume (z-stack) images [400(x)×400(y)×100(z) am], spanning the basal layer of the corneal epithelium 46 to the mid-stroma 48.

The microscopy images 20 may be acquired at a plurality of time points at a predetermined frequency over a time period to provide a plurality of microscopy images 20 at each of a series of time points. The predetermined frequency may be in the range of about 2 minutes to about 9 minutes, preferably in the range of about 4 minutes to about 7 minutes, and more preferably in the range of about 4 minutes to about 5 minutes. Each time point may comprise a time range of more than 0 minutes to about 2 minutes. Thus, the time point may not represent a specific time, e.g., 3 minutes, but may encompass a time range. The time period may be in the range of about 20 minutes to about 60 minutes, and preferably in the range of about 15 minutes to about 25 minutes, and more preferably, the time period is no more than about 40 minutes. A series of about 5 time points to about 10 time points may be provided based on the predetermined frequency and time period. En face images 22 at the same tissue plane may be extracted from the volume scans 20, registered to stationary landmarks 68, and time-lapsed videos 24, 26 may be reconstructed at both the level of the corneal epithelium 46 and across the depth of the stroma 48.

### Inventive IVCM Method for Depth Imaging

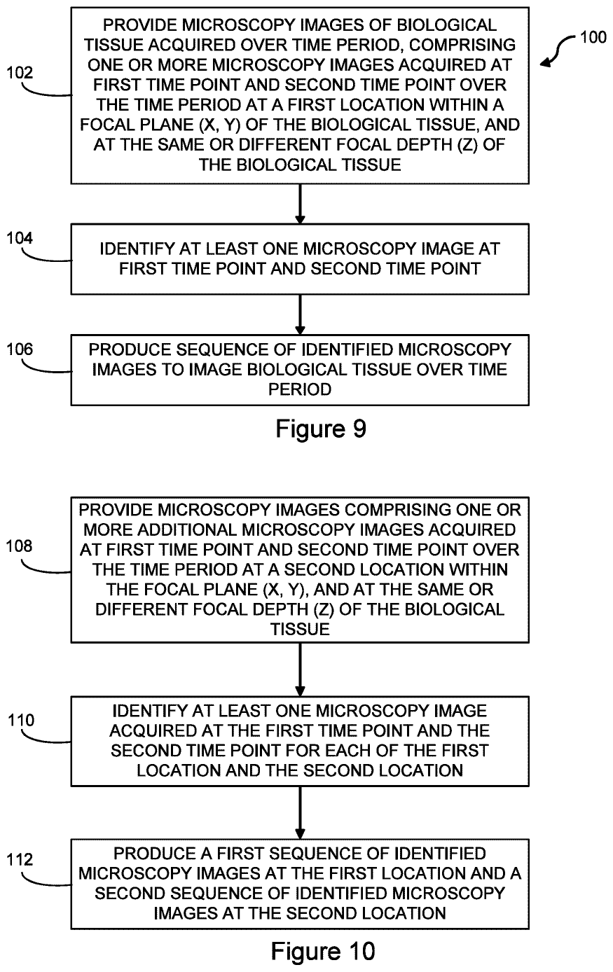

Embodiments of the disclosure providing for depth imaging, such as the example in FIG. 8 of the human cornea, will now be described with reference to FIGS. 9 to 14.

FIG. 9 is a flow chart of a method 100 for imaging a biological tissue 40 over time, according to some embodiments of the disclosure. The method comprises at step 102 providing microscopy images 20 of the biological tissue 40 acquired over a time period. The microscopy images 20 provided comprise one or more microscopy images 20 acquired at a first time point over the time period, where the one or more microscopy images 20 are acquired at a first location 50 within a focal plane (x, y) of the biological tissue 40 and at a focal depth (z) of the biological tissue 40. The microscopy images 20 provided also comprise one or more microscopy images 20 acquired at a second time point over the time period, where the one or more microscopy images 20 are acquired at the first location 50 within the focal plane (x, y) of the biological tissue 40 and at the same or a different focal depth (z) of the biological tissue 40. The method 100 also comprises the step 104 of identifying at least one microscopy image 22 acquired at the first time point and the second time point. The method 100 also comprises the step 106 of producing a sequence of the identified microscopy images 22, thereby imaging the biological tissue over the time period. The sequence may then be used to generate time-lapsed videos or combined overlaid images to analyse and/or present changes in one or more features of the biological tissue over the time period.

In some embodiments, the at least one microscopy image 22 is identified for use in the sequence based on a desired focal depth (z) for imaging of the biological tissue 40. For example, the at least one microscopy image 22 may be identified for each of the first time point and the second time point based on a focal depth within a particular tissue layer of the biological tissue, e.g., an epithelial 46 or stromal layer 48 of the cornea 40 in the embodiments discussed herein. The desired focal depth (z) may be a range of depths representative of the corneal epithelium 46 or the corneal stroma 48. The at least one microscopy image 22 may be identified based on the same focal depth (z) at the first time point and the second time point, e.g., both within the epithelium 46 or stroma 48 for imaging at the first location 50.

In order to perform volume scanning at depth, the method 100 may comprise at step 102 of providing microscopy images 20 comprising a plurality of microscopy images acquired at the first time point and the second time point for the first location 50 across a predetermined range of focal depths (z) of the biological tissue. A volume scan may thus be acquired at each of the first time point and the second time point for the first location 50 at a range of focal depths (z).

In other embodiments of the disclosure, a sequential scan at depth is also possible using the method 100 as disclosed herein. The method 100 may comprise at step 102 of providing microscopy images 20 comprising a plurality of microscopy images acquired at the first time point and the second time point, where the images are a series of repeated images acquired at the first location 50 within the focal plane (x, y) at the focal depth (z) of the biological tissue 40. The series of repeated images may comprise about 2 images to about 100 images, for example. The repeated images may be acquired at the first location 50 at or about the same position within the focal plane (x, y). In other embodiments, the repeated images may be acquired at the first location 50 but at different positions within the focal plane (x, y). This may involve an operator or programmed system either moving the objective 14 of the microscope 10 to capture adjacent tissue areas or have the subject 30 move their eyes 32 to capture these adjacent areas, e.g., by looking at specific markers. A sequential scan may thus be acquired at each of the first time point and the second time point for the first location 50 at the same position or different positions at a particular focal depth (z).

FIG. 10 is a flow chart showing additional steps in the method 100 of FIG. 9 relating to providing additional microscopy images at a second location 52 and either at the same or different focal depth (z) of the biological tissue 40, according to some embodiments of the disclosure. The method 100 may comprise at step 108 providing one or more additional microscopy images at the first time point and/or the second time point. The one or more additional microscopy images at the first time point may be acquired at a second location 52 within the focal plane (x, y) of the biological tissue 40 and at the focal depth (z) of the biological tissue 40. The one or more additional microscopy images at the second time point may be acquired at the second location 52 within the focal plane (x, y) of the biological tissue 40 and at the same or a different focal depth (z) of the biological tissue 40. The step 104 of identifying at least one microscopy image may comprise the step 110 of identifying at least one microscopy image 22 acquired at the first time point and the second time point for each of the first location 50 and the second location 52. The method 100 may further comprise the step 112 of producing a first sequence of the identified microscopy images 22 at the first location 50 and a second sequence of the identified microscopy images 22 at the second location 52.

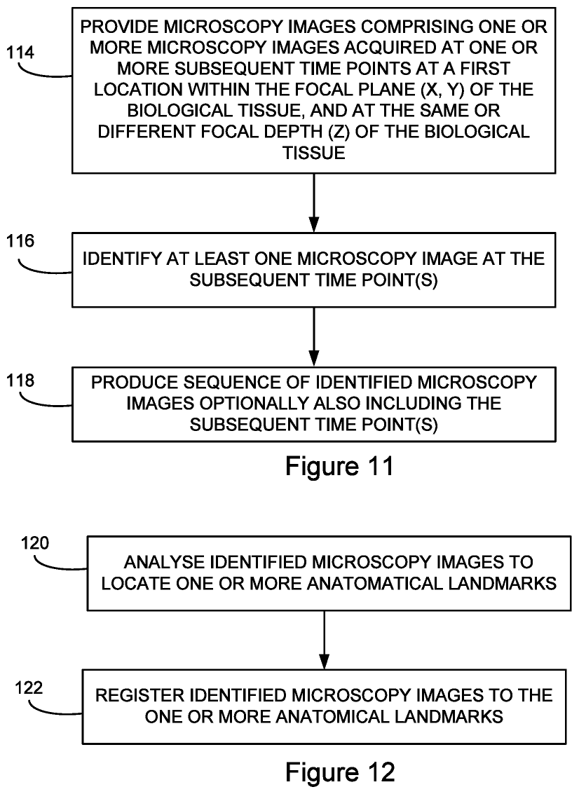

FIG. 11 is a flow chart showing additional steps in the method 100 of FIG. 9 relating to providing microscopy images 20 acquired at one or more subsequent time point(s) at the first location 50, according to some embodiments of the disclosure. The step 102 of providing microscopy images 20 may further comprise providing at step 114 one or more microscopy images 20 acquired at one or more subsequent time points over the time period, where the one or more microscopy images 20 are acquired at the first location 50 within the focal plane (x, y) of the biological tissue 40 and at the same or a different focal depth (z) of the biological tissue 40 to the first and second time points. The step 104 of identifying the at least one microscopy image 22 may further comprise the step 116 of identifying at least one microscopy images 22 acquired at the one or more subsequent time points. Furthermore, the method 100 may optionally include at step 118 producing a sequence of the identified microscopy images 22 also including the one or more subsequent time points. Steps 108 and 110, and 116 and 118 may include similar aspects to those described in relation to steps 104 and 106 from FIG. 9.

FIG. 12 is a flow chart showing additional steps in the method 100 of FIG. 9. The method 100 may comprise at step 120 analysing the identified microscopy images 22 to locate one or more anatomical landmarks 68 and at step 122 registering the images 22 to the landmarks 68, according to some embodiments of the disclosure. The method 100 may further comprise at step 104, identifying the at least one microscopy image 22 for each time point based on an anatomical landmark. The method 100 may further comprise a step of assigning a location identifier to the identified images 22 having the same anatomical landmark. In various embodiments where a plurality of microscopy images 20 are acquired, a location identifier may be assigned to each of the identified images 22 having the same anatomical landmark. The location identifier may be e.g., a numerical or alphabetical code assigned to the image, indicative of an anatomical landmark. The location identifier may be used in the method 100 for ordering of the images 22 into a sequence and/or display of the images 22, and/or determining the presence of at least one object of the biological tissue 40 such as for tracking of the object (e.g., a cell).

The anatomical landmark may include one or more of a nerve, a pattern of nerves, a cell, a pattern of cells, and at least one feature of connective tissue. In particular, the anatomical landmark may include a stationary cell(s) or pattern of stationary cells in the identified images 22. The anatomical landmark may include a whorl region 68 of the corneal nerves 40 as shown in FIG. 3. In other embodiments, the anatomical landmark may include nerve axons as static features to register the images 22 at step 122. Additionally/alternatively, the anatomical landmark may include stationary keratocyte nuclei for imaging of the cornea 40. The location identifier may include a depth of the biological tissue 40 at the location 50, 52. The location identifier may be a layer in the tissue, such as the epithelium or stoma of the cornea 40.

Advantageously, the registration of the identified images 22 at step 122 for each time point being based on an anatomical landmark and/or assignment of a location identifier enables an operator to find the same region of tissue or location 50, 52 for imaging over a time period. This is desirable as it can be difficult to find the same region for imaging due to movement of the subject 30 in between acquisitions over the time period. It can be challenging to re-align the subject 30 using the head rest 12 of microscope 10 for imaging of the cornea 40 as shown in FIGS. 1 to 8 also once the subject 30 has moved from their initial position. This can result in the size of the image region for analysis decreasing because the same overlapping region of tissue or location 50, 52 cannot be easily identified. Embodiments of the disclosure may advantageously provide additional microscopy images 20 acquired at each of the time points using volume scans to account for these movements and to enable the same region of tissue or location 50, 52 to be located in each of the identified images 22 for analysis.

The sequence of identified microscopy images 22, which may comprise a first sequence and/or a second sequence, may consist of an ordered sequence of the identified microscopy images. The ordering may be based on time of acquisition of the microscopy images 20. The ordering may or may not be sequential. Additionally/alternatively, the ordering may be based on at least one feature of the biological tissue 40 in the microscopy images 22 e.g., an object or cell to be tracked through the images 22, or optionally, one or more anatomical features of the biological tissue 40.

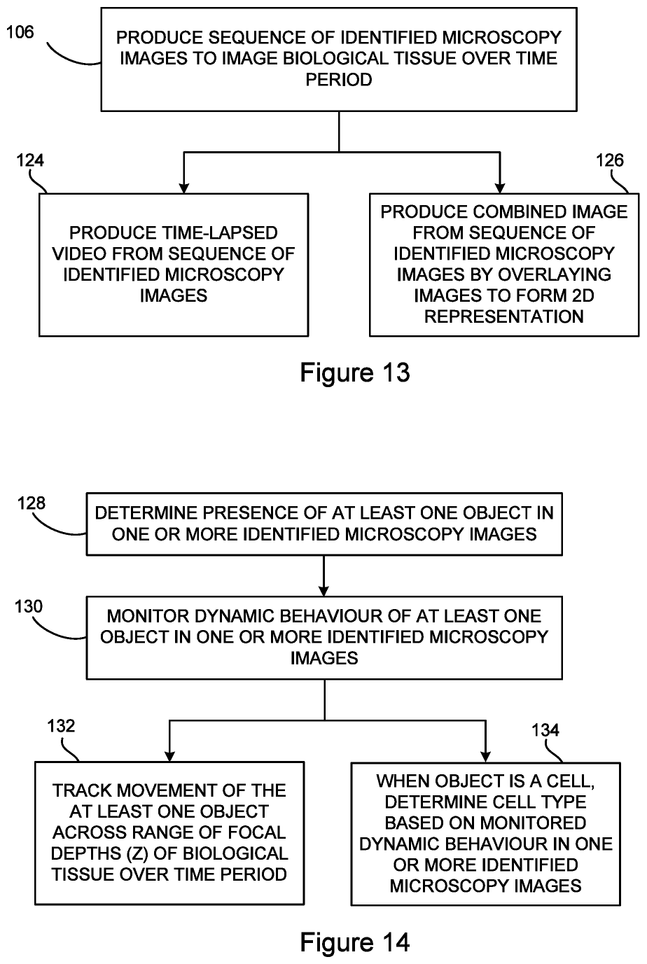

FIG. 13 is a flow chart showing additional steps in the method 100 of FIG. 9 relating to producing a time-lapsed video and a combined image of the sequence of identified microscopy images 22 to image the biological tissue 40. After the step 106 of producing the sequence, the method 100 may further comprise the step 124 of producing a time-lapsed video from the sequence of the identified microscopy images 22 at a desired frame rate. The desired frame rate may be in range of about 1 to about 10 frames/second, preferably about 1 to about 5 frames/second, and more preferably about 3 frames/second.

Additionally/alternatively, the method 100 may further comprise the step 126 of producing a combined image 26 from the sequence of the identified microscopy images 22 by overlaying the images to form a two-dimensional (2D) representation. The combined image 26 may use one or more colours representing changes to one or more features of the biological tissue 40 over the time period (e.g., changes in activity and/or dynamic behaviour of at least one object, such as a cell). The combined image 26 may be a time-lapsed image.

FIG. 14 is a flow chart showing additional steps in the method 100 of FIG. 9 relating to determining the presence of at least one object 60, e.g. a cell, in one or more of the identified microscopy images 22, monitoring dynamic behaviour of the object 60 and then optionally tracking movement of the object 60 across a range of focal depths (z) and/or when the object 60 is a cell, determining cell type, according to some embodiments of the disclosure.

At step 128, the method 100 may comprise determining the presence of at least one object 60 in one or more of the identified microscopy images 22 of the biological tissue over the time period. It will be appreciated that depending on the application, a person may identify the object but equally, depending on the application, an automated tool may be utilised to identify the object. The at least one object 60 may be static over the time period, and the presence of the at least one object 60 may be determined based on one or more characteristics. The characteristics may be selected from at least one of a group comprising: location 50, 52 within the focal plane (x, y) of the biological tissue 40, focal depth (z) of the biological tissue 40, reflectivity, and one or more morphological parameters. As previously discussed, the one or more morphological parameters may be selected from at least one of a group comprising: object field area, object area, object perimeter, object circularity, object roundness, object solidity, and dendritic complexity. The at least one static object 60 may be a non-immune cell. The non-immune cell may be a fibroblast. Where the biological tissue 40 is corneal tissue, the static object 60 may be a corneal keratocyte.

In other embodiments, the object 60 may be dynamic over the time period. The presence of the at least one object 60 may be determined based on one or more characteristics. The characteristics may be selected from at least one of a group comprising: location 50, 52 within the focal plane (x, y) of the biological tissue 40, focal depth (z) of the biological tissue, reflectivity, one or more morphological parameters, and one or more dynamic parameters. As previously discussed, the one or more morphological parameters may be selected from at least one of a group comprising: object field area, object area, object perimeter, object circularity, object roundness, object solidity, and dendritic complexity. Where the object 60 is a cell, the one or more morphological parameters may comprise field area, cell area and cell solidity. The reflectivity may be a measure of hyper-reflectivity of the object.

The density and morphology of the object(s) in the microscopy images of the biological tissue, e.g., one or more cells or cell types within tissue, may be determined by the following image processing techniques:
1. Identify immune cells in microscopy images and label cells so that they can be tracked and matched.
2. To quantify cell density, count the number of moving cells either by eye or using a cell counter function (e.g., ImageJ Plugins-analyse-Cell counter).
3. In some embodiments, the images may be of original size, for example, the area of the image will be 16000 μm² or more. For time-lapsed videos, this rarely happens due to imperfect alignment and so most images will need the area calculated to compensate for loss of area. To calculate the area, the microscopy images need to be calibrated (1 micron=0.96 pixels). For original image size, 384×384 pixels=400 um×400 μm. When the image stack is duplicated, the dimensions of the image will be automatically determined, and the multiplication factor can be calculated.
4. Count the cells (e.g., immune cells and keratocytes as desired) and calculate the density based on the multiplication factor.

To measure cell morphology and various parameters, the image processing steps may involve one or more of:
1. Measuring the polygon field area by selecting the first frame of images for all cells. The polygon can be created from tip-to-tip.
2. For the remaining morphology parameters, measure each cell starting at the first frame, manually trace the cell e.g., using the “freehand selection” in ImageJ.
3. Create a mask based on the trace of the cell. Repeat for each of the cells in the image frame.
4. Once all cells have been traced at each time point and all masks created, complete morphology measurements for each cell that is matched (pre-post comparisons).
5. Calculate the average parameter for each cell across each time point, including area, perimeter, Feret's diameter, circularity, roundness and solidity.

For dynamic objects 60, the method 100 may further comprise the step 130 in FIG. 14 of monitoring the dynamic behaviour of the at least one object 60 in one or more identified microscopy images 22 of the biological tissue 40 over the time period. The step 130 may comprise monitoring one or more dynamic parameters of the at least one object over the time period. The one or more dynamic parameters may be selected from at least one of a group comprising: instantaneous speed, arrest coefficient, object displacement speed, meandering index, displacement ratio, linearity of forward progression, mean directional change, dendrite surveillance extension and retraction cycling habitude (dSEARCH), probing speed, outreach ratio, and dancing index.

The method 100 of FIG. 9 may further comprise the step 132 of tracking movement (e.g., dynamic motion) of the at least one object 60 (e.g., a cell) across a range of focal depths (z) of the biological tissue 40 over the time period. Where the object 60 is a cell, the method 100 may further comprise the step 134 of determining the cell type based on the monitored dynamic behaviour of the at least one cell in the one or more identified microscopy images 22 of the biological tissue 40 over the time period. The at least one dynamic object 60 may be an immune cell. The cell type may be selected from one of a group comprising: a putative T cell, a putative dendritic cell, or a putative macrophage.

An example of the image processing for tracking of movement of an object (e.g., a cell) may include the following steps:
1. Identify the object, e.g., a cell, desired for tracking in the first image frame of the sequence of images (e.g., in the time-lapsed video or combined colour image) using image processing software such as ImageJ (Plugins-Tracking-Manual Tracking).
2. Review adjacent image frames to determine direction of movement of the object.
3. Add track to the cell in the first image frame. Move through the image frames and identify the cell in each of the images.
4. Optionally produce a tracking video of the sequence of images showing the overlayed dots & lines.

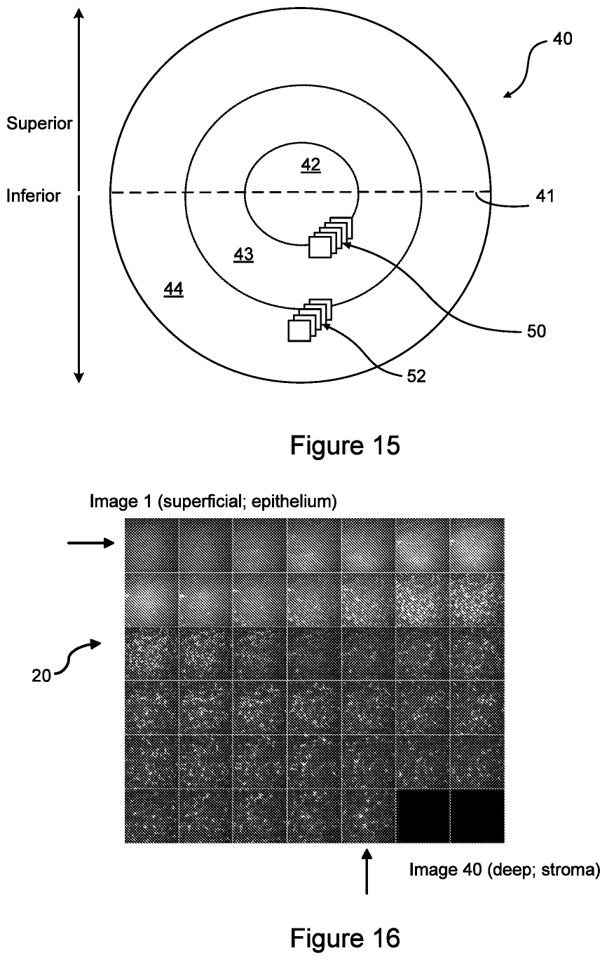

FIG. 15 is a schematic of a cornea 40 of the subject 30 showing two indicative/representative locations 50, 52 for depth imaging using the method 100 of FIG. 9, according to some embodiments of the disclosure. Although two locations 50, 52 are shown, the microscopy images may be acquired at any number of locations in the focal plane (x, y) of the cornea 40 and at any number of positions in the focal plane (x, y) which are not necessarily limited to the specific ones shown in the Figures. The method 100 may comprise providing microscopy images 20 acquired at each of the two locations 50, 52 over the time period. The two locations 50, 52 may include the paracentral cornea 43 and the peripheral cornea 44 as illustrated in FIG. 15. In some embodiments, one or more additional microscopy images are acquired at each of the two locations 50, 52 over the time period. The number of additional images at each location depends on the acquisition rate which is defined by a predetermined frequency over the time period.

FIG. 16 is an example of a plurality of microscopy images 20 acquired across a depth of the corneal stroma 48 at a first location 50 according to the method 100 of FIG. 9, according to some embodiments of the disclosure. In this case, the plurality of microscopy images 20 includes 40 images which were acquired across a predetermined depth of the biological tissue 40 including from the epithelium 46 (superficially) to the stroma 48 (deep within the cornea 40). Moving from Image 1 to Image 40 it is evident that the x-y enface planar images vary in population of cells and other structures in the region of tissue at the first location 50. The changing composition of the images through the depth of the tissue will be discussed in more detail below.

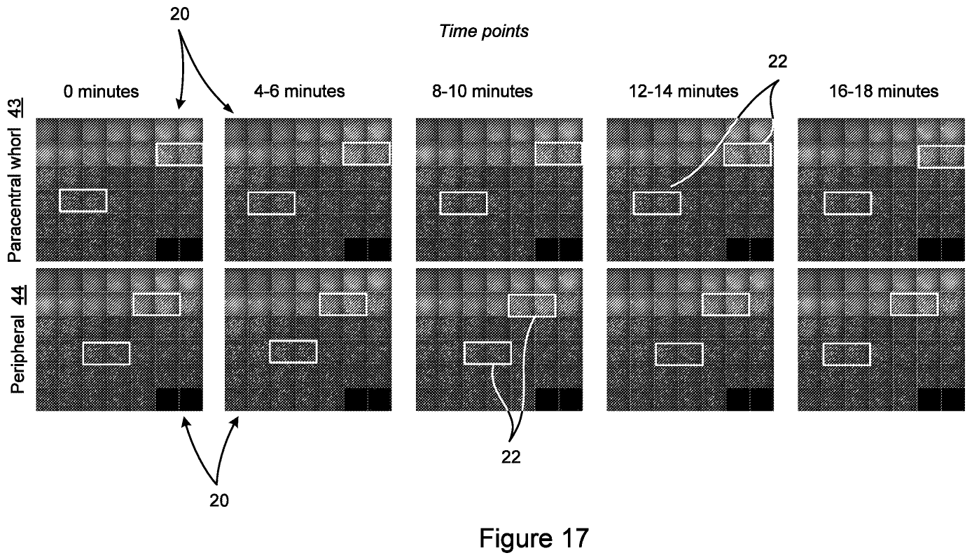

FIG. 17 is a plurality of microscopy images 20 acquired across a depth of the cornea 40 at two locations, a first location being the paracentral whorl 43 and a second location being the peripheral cornea 44, at a series of time points, and demonstrating the steps 104, 110 of identifying at least one microscopy image 22 at each of the time points according to embodiments of the method 100 of FIG. 9. The time period for acquisition of the images is 20 minutes in this example, with the predetermined frequency of about 4 to about 5 minutes for image acquisition over the time period. In this case, microscopy images 20 at each of the two locations 50, 52 in the cornea 40 have been acquired at 5 time points including at the time points of 0 minutes, 4-6 minutes, 8-10 minutes, 12-14 minutes and 16-18 minutes.

In order to analyse cell behaviour, two images 22 can be identified at each of two depths in the microscopy images 20 for each time point. This is evident by the boxes 22 which each include two adjacent images. In this case, the two microscopy images 22 have been identified based on the same focal depth (z) (out of the images 1 to 40) for each of the time points. Based on the identified images 22, a sequence of the images can be produced. Furthermore, a time-lapsed video can be produced using the sequence of images or a combined image overlaying the identified images, which may be in false colour. This may enable observation of dynamic cells such as putative macrophages in the stroma 48 of the cornea 40.

In order to produce the time-lapsed video or combined image, the identified images 22 must undergo a registration process to align the images with regard to orientation, position and/or size. An example of the image processing for image registration for volume scans may include the following steps:
1. Provide volume scans which include a plurality of microscopy images.
2. Calibrate each image to a known scale, which in this example are image sizes of 400 μm×400 μm.
3. Analyse each image frame to identify one or more anatomical landmarks beginning anteriorly and moving posteriorly. Optionally, label each image frame having an anatomical landmark with a location identifier.
4. Duplicate image frames that appear to be in similar regions, e.g., at or about the same position in the focal plane (x, y) for each of the time points or at or about the same focal depth (z) for each of the time points. The identified image frames are saved as a sequence of images, e.g., sorted in chronological order or based on a feature of the biological tissue, e.g., an anatomical landmark, or identified object.
   If two adjacent frames are slightly out of the plane of focus and are substantially aligned, the image frames are stacked together in order to input more information into the image. If two volume scans are collected in the same location at about the same timepoint (separated for example, by a few seconds as opposed to a larger interval of, for example, 3-4 minutes), analyse each image frame in the subsequent volume scan to identify the same anatomical landmarks and locate e.g., the same cells. Two adjacent image frames from the two volume scans can be stacked if acquired at the same time point.
5. Create an image stack of the duplicated image frames in the sequence of identified images.
6. Register the image stack.
7. If colour-coded visualisation is desired, a hyperstack can be created.
8. The frame rate of the time-lapsed video or combined image can be altered as desired.

Thus, it is possible to capture using embodiments of the inventive IVCM method 100 different immune cell sub-classes through the depth of the same tissue region, which has never been described before using IVCM. This will be described in more detail in the Examples, where immune cells may be identified in the images including putative T cells, putative dendritic cells and putative macrophages. Significantly, the method 100 may enable the capture of migration of individual cells through the depth of the tissue. For example, a putative T cell travelling from the corneal epithelium 46 into the stroma 48 over the time period, or a cell travelling from the anterior stroma to deeper within the stroma 48. That is, embodiments of the disclosure have utility in dynamically tracking cells, specifically immune cells, over the corneal tissue depth in living human eyes, over time.

### New IVCM Method Using Sequential Imaging

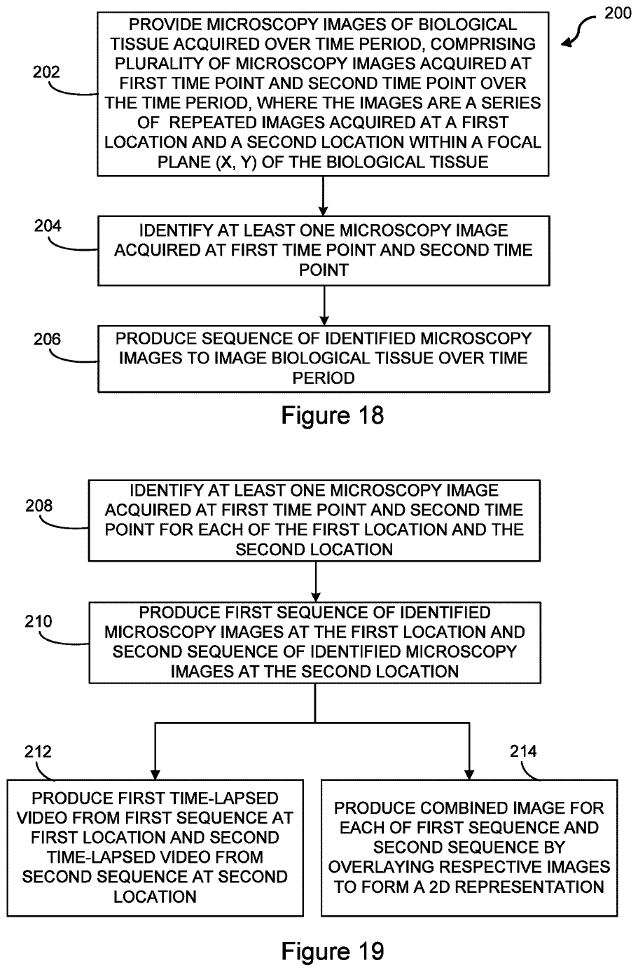

Embodiments of the disclosure providing for sequential imaging at two or more locations will now be described with reference to FIGS. 18 to 23. Where similar steps are described from the method 100 of FIGS. 9 to 17, such as identifying at least one microscopy image and producing a sequence of the identified microscopy images, the various features have not been described again in detail or completely for conciseness in relation to the method 200 described below unless those features are differently applied.

FIG. 18 is another method 200 for imaging biological tissue 40 over time, according to some embodiments of the disclosure. The method 200 comprises the step 202 of providing microscopy images 20 comprising a plurality of microscopy images acquired at a first time point over the time period, where the plurality of microscopy images are a series of repeated images acquired at a first location 50 and a second location 52 within a focal plane (x, y) of the biological tissue 40. The step 202 further comprises providing microscopy images 20 comprising a plurality of microscopy images acquired at a second time point over the time period, where the plurality of microscopy images is a series of repeated images acquired at the first location 50 and the second location 52 within the focal plane (x, y) of the biological tissue 40. The method 200 also comprises the step 204 of identifying at least one microscopy image 22 acquired at the first time point and the second time point. The method 200 also comprises the step 206 of producing a sequence of the identified microscopy images 22, thereby imaging the biological tissue 40 over the time period.

The method 200 may be for monitoring dynamic cell behaviour in the epithelium 46 of the cornea 40. For epithelial immune cells, depth is not as useful as the cells are mostly located within a 5-10 micron focal plane. A sequence or sequential scan is useful for imaging of the cells. Larger areas may be covered by instructing the subject 30 to move their gaze along guided wall-mounted targets, or by an operator manually moving the microscope objective 14 during acquisition. The sequence scan may generate 100 frames and nerve patterns or other similar anatomical landmarks (as described with reference to the method 100) may be used to guide the operator to find the same region of tissue or location 50 for each acquisition.

FIG. 19 is a flow chart showing additional steps in the method 200 of FIG. 18 relating to producing a time-lapsed video 24 and a colour time-lapsed merge 26 of the identified microscopy images 22 to display cellular activity and/or dynamic behaviour of at least one object 60, e.g., a cell, at each of the two or more locations 50, 52. The method 200 comprises the step 208 of identifying at least one microscopy image 22 acquired at the first time point and the second time point for each of the first location 50 and the second location 52. After the step 208, the method 200 may further comprise the step 210 of producing a first sequence of the identified microscopy images 22 at the first location 50, and a second sequence of the identified microscopy images 22 at the second location 52. The sequence of identified images 22 may be used to form a time-lapsed video from the first sequence and from the second sequence at step 212 depending on the frame rate. Additionally/alternatively, the method 200 may further include the step 214 of overlaying the identified images 22 to form a two-dimensional (2D) representation to produce a combined image 26 for each of the two or more locations 50, 52. The combined image 26 may use one or more colours to represent changes in one or more features of the biological tissue, e.g., cellular activity and/or dynamic behaviour of the at least one object, e.g., a cell. Thus, the method 200 enables a time-lapsed video 24 or colour combined image 26 to be created for each of the two locations 50, 52 of the biological tissue 40.

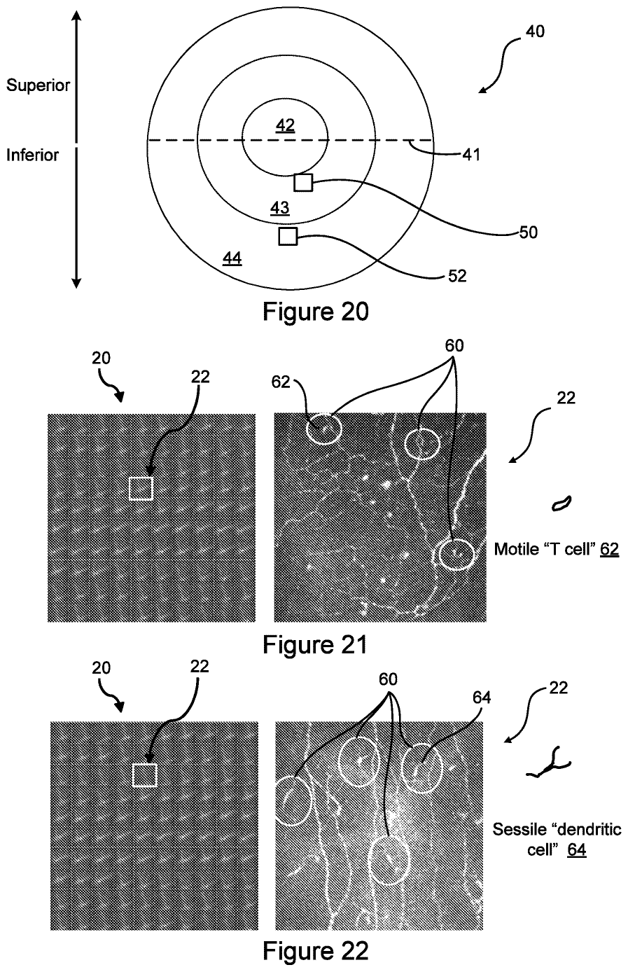

FIG. 20 is a schematic of a cornea 40 of the subject 30 showing two indicative/representative locations 50, 52 for sequential imaging according to some embodiments of the disclosure. Although two locations are shown, the present disclosure is not limited to two locations for sequential imaging and images at any number of locations in the focal plane (x, y) may be acquired. In this method 200, the two or more locations 50, 52 may be at the same depth in the region of tissue 50. That is, the two locations 50, 52 may be in the same tissue layer, such as in the epithelium 46 or stroma 48 of the cornea 40. The two locations may include a paracentral region 43 and a peripheral region 44 of the cornea 40 of the subject 30.

FIGS. 21 and 22 illustrate sequential microscopy images 20 acquired at the two locations, namely the paracentral cornea 43 (FIG. 21) and the peripheral cornea 44 (FIG. 22), identifying one image 22 for analysis (see white box in left image), and identifying at least one cell in the image (see circled cell regions 60 in right image). In FIG. 21, a putative motile “T cell” 62 has been identified in each of the three circled cell regions 60. In FIG. 22, a putative sessile “dendritic cell” 64 has been identified in each of the four circled cell regions 60. The sequential microscopy images 20 may be acquired by a sequence scan using microscope 10 to acquire 100 images which are captured over a short time frame at or about the same locations 50, 52. For example, the sequence scan may capture 100 frames from each of the two locations 50, 52 every 3-4 minutes over a time period of 20 minutes. The inventors have shown that collection of images from two locations 50, 52 in the epithelium 46 of the cornea 40 will reveal two major immune cell types, putative T cells 62 and putative dendritic cells 64. The putative motile T cells 62 are mostly located in the central cornea 42 at the “whorl region” 68. Sessile dendritic cells 64 tend to be located in the peripheral cornea 44. This described in more detail in the Examples.

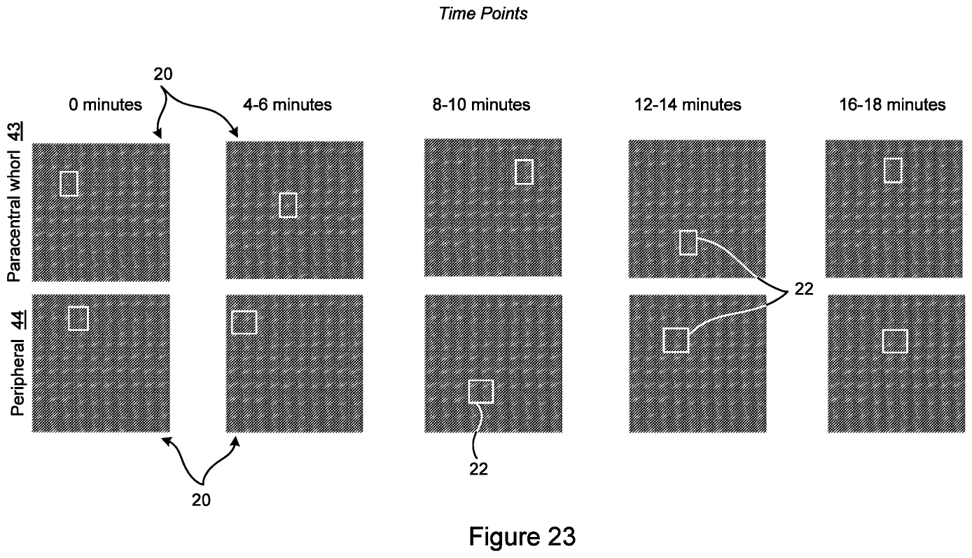

FIG. 23 is a series of microscopy images 20 acquired sequentially at each of two locations, the paracentral whorl 43 and the peripheral cornea 44, at a series of time points over a time period, according to some embodiments of the disclosure. The time period for acquisition of the images is 20 minutes in this example, with the predetermined frequency of about 4 to about 5 minutes for image acquisition over the time period. In this case, 5 groups of microscopy images 20 have been captured for each of the two locations 50, 52 in the cornea 40 at the time points of 0 minutes, 4-6 minutes, 8-10 minutes, 12-14 minutes and 16-18 minutes.

In order to analyse cell behaviour, one image 22 can be identified from the microscopy images 20 acquired at each time point for each of two locations 42, 44. This is evident by the boxes 22 which each include one image 22 for each location 42, 44. Based on the retrieved images 22, a time-lapsed video 24 can be generated with the sequence of images or a colour time-lapsed video 26 which overlays the images. Advantageously, collection of data from two locations increases the total number of cells and the number of cell subsets that can be analysed. By examining data from multiple locations (e.g., two or more locations), it may be possible to analyse changes to immune cells in different corneal regions which might be caused by exposure to different types of conditions, stimuli and/or therapeutics. For example, a systemic drug might affect all corneal regions equally, whereas a contact lens might preferentially affect different regions of the cornea as determined by the thickness profile of the lens, or different drugs might differentially modulate subtypes of immune cells which have differing density across the corneal eccentricity. Imaging more locations also increases the likelihood of capturing more cell-to-cell and cell-to-nerve interactions.

### Assessing Object-Object Interactions

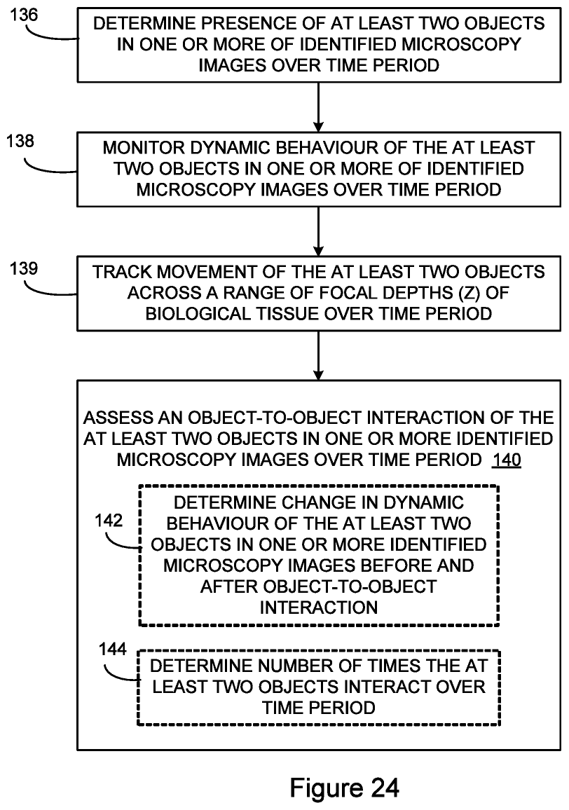

FIG. 24 is a flow chart showing additional steps in the method 100 of FIG. 9 or the method 200 of FIG. 18 relating to determining the presence of at least two objects and assessing an object-to-object interaction, according to some embodiments of the disclosure.

The at least two objects may be cells. The object-to-object interaction may be a cell-to-cell interaction. The at least two objects may be immune cells. In some embodiments, the two cells are different cell types. Alternatively, the two cells may be the same cell type. The cell type may be selected from one of a group comprising: a putative T cell, a putative dendritic cell, or a putative macrophage.

The methods 100, 200 may further comprise the step 136 of determining the presence of at least two objects in one or more of the identified microscopy images 22 of the biological tissue over the time period. The presence of the at least two objects may be determined based on one or more characteristics, namely the previously mentioned characteristics for dynamic objects, e.g., motile or moving objects. The methods 100, 200 may further comprise the step 138 of monitoring dynamic behaviour of the at least two objects in one or more of the identified microscopy images 22 of the biological tissue over the time period.

The methods 100, 200 may further comprise the step 139 of tracking movement of the at least two objects across a range of focal depths (z) of the biological tissue 40 over the time period.

The methods 100, 200 may further comprise the step 140 of assessing an object-to-object interaction of the at least two objects in one or more of the identified microscopy images 22 over the time period. The object-to-object interaction may be quantified based on how dynamic behaviour of the objects change after the interaction and/or based on the number of events, such as the percentage of objects forming a spatial interaction over the time period. The step 140 of assessing the object-to-object interaction may include one or both of the steps 142 and 144 as shown in FIG. 24. Step 142 may include determining a change in the dynamic behaviour of the at least two objects in one or more of the identified images 22 before and after the object-to-object interaction. This may include analysing the dynamic behaviour of the two objects such as by quantifying one or more dynamic parameters, e.g., set out in Table 1 in the Examples and imaging processing as described in the Materials and Methods section. Step 144 may include determining the number of times that the two objects interact over the time period.

When the objects are cells, the cell-to-cell interaction may be quantified based on a number of biologically significant parameters. For example, the duration of the cell-to-cell interaction, changes in directionality of movement after the cell-to-cell interaction, change in dendrite orientation after the cell-to-cell interaction, and changes in cell morphology after the interaction.

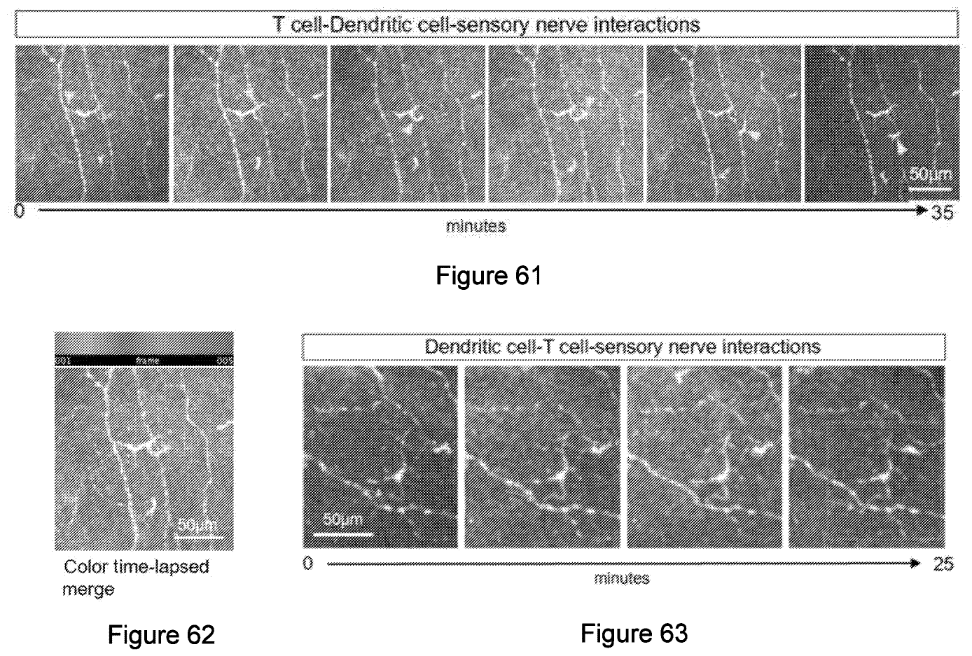

Embodiments of the disclosure may be used to quantify T cell-Dendritic cell interaction and T cell-T cell interactions for immune cells in the cornea 40 of the subject 30. This is described in more detail with reference to Example 1D and FIGS. 61 to 65.

### Assessing Object-Nerve Interactions

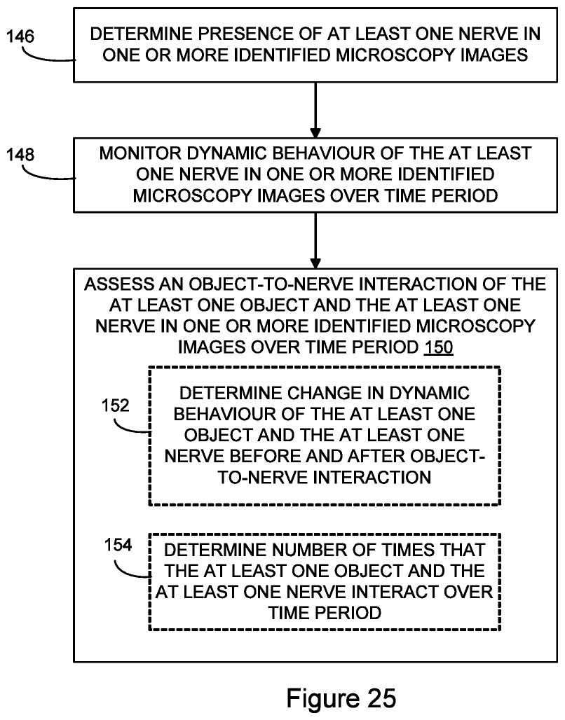

FIG. 25 is a flow chart showing additional steps in the method 100 of FIG. 9 or the method 200 of FIG. 18 relating to determining the presence of at least one nerve and assessing an object-to-nerve interaction, according to some embodiments of the disclosure. The object may be a cell. The object-to-nerve interaction may be a cell-to-nerve interaction. The object may be an immune cell. The cell type may be selected from one of a group comprising: a putative T cell, a putative dendritic cell, or a putative macrophage.

The methods 100, 200 may further comprise the step 146 of determining the presence of at least one nerve in one or more of the identified microscopy images 22. As will be appreciated this may be by way of appearance or morphology. For example, a nerve might appear white in colour (high reflectivity) on IVCM, and have a relatively linear or curvilinear pattern (depending on the region of cornea being imaged), with central fibres from which nerve branches derive. The methods 100, 200 may further comprise the step 148 of monitoring dynamic behaviour of the at least one nerve in one or more of the identified microscopy images 22 over the time period.

The methods 100, 200 may further comprise the step 150 of assessing an object-to-nerve interaction of the at least one object and the at least one nerve in one or more of the identified microscopy images 22 over the time period. The object-to-nerve interaction may be quantified based on how the dynamic behaviour changes after the interaction and/or based on the number of events, such as the percentage of cells forming a spatial interaction over the time period. The step 150 of assessing the object-to-nerve interaction may comprise one or both of the steps 152 and 154 as shown in FIG. 25. Step 152 may include determining a change in dynamic behaviour of the at least one object and the at least one nerve before and after the object-to-nerve interaction. This may include analysing the dynamic behaviour of the object and nerve such as by one or more dynamic parameters, e.g., out in Table 1 in the Examples and imaging processing as described in the Materials and Methods section. Step 154 may include determining the number of times that the object and nerve interact over the time period.

When the objects are cells, the cell-to-nerve interaction may be quantified based on a number of biologically significant parameters. For example, the duration of the cell-to-nerve interaction, changes in directionality of movement after the cell-to-nerve interaction, change in dendrite orientation after the cell-to-nerve interaction, and changes in cell morphology after the interaction.

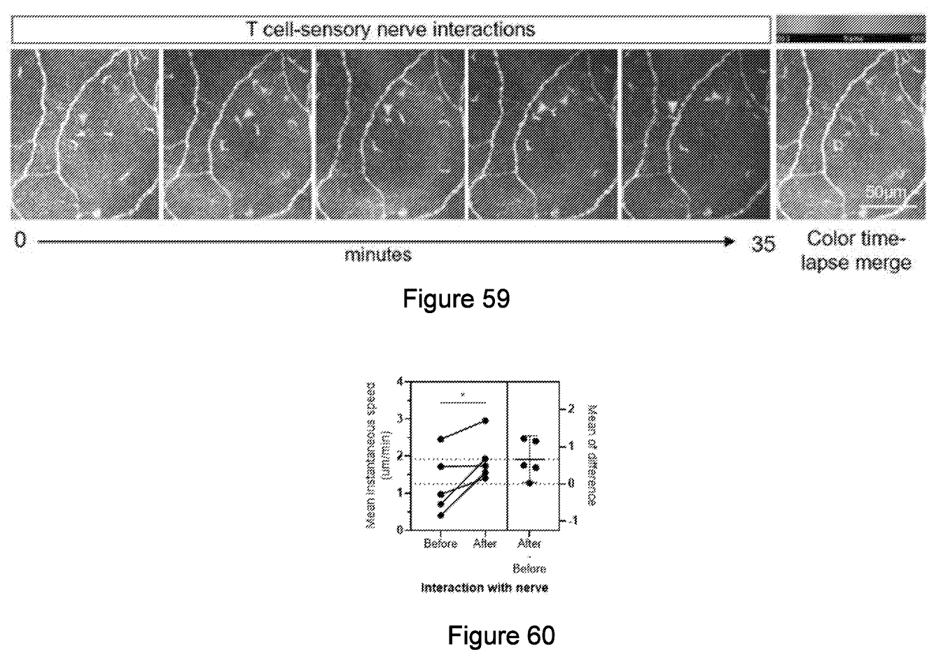

Embodiments of the disclosure may be used to quantify T cell-nerve interactions and Dendritic cell-nerve interactions for immune cells in the cornea 40 of the subject 30. This is described in more detail with reference to Example 1D and FIGS. 59 to 65.

Significantly, dynamic spatial in vivo cell-to-cell interactions and in vivo cell-to-nerve interactions have never before been assessed in subjects, human or animal. Using the new imaging methods 100, 200 and further described with reference to FIGS. 24 and 25 for various embodiments of the disclosure, it is possible to measure the amount of time or degree of interaction between two cells, or between a cell and a nerve. The capacity to visualise these interactions in living subjects provides for the ability to look at the effects of different conditions, agents or exposures in modifying these functional interactions. For example, it may be that in a certain condition, the neuro-immune response is impaired, leading to reduced capacity to fight corneal infections or for the cornea to not heal properly after injury. Thus, the inventive technique permits direct visualisation of complex functional biological phenomena that could provide insight into the pathophysiology of a disease, or the mechanisms of action of treatment, or be used as a diagnostic or prognostic biomarker etc.

The capacity to capture these fundamental interactions between the nervous and immune systems in the living human eye using the new IVCM technique provides opportunities to study the interface between nerves and immune cells in an intact peripheral sensory tissue, including how exogenous factors and disease may disrupt the perception, integration and responsiveness of cell subtypes to different challenges.

### Matched Cell Analysis

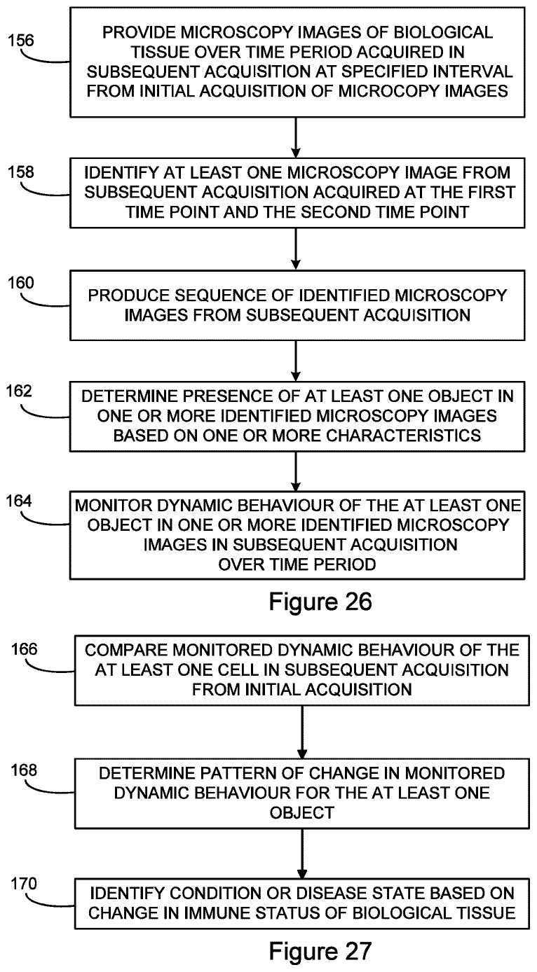

FIG. 26 is a flow chart showing additional steps in the method 100 of FIG. 9 or the method 200 of FIG. 18 relating to providing microscopy images 20 acquired at a subsequent acquisition at a specified interval from the initial acquisition at the first location, according to some embodiments of the disclosure.

The methods 100, 200 further comprise the step 156 of providing microscopy images 20 of the biological tissue 40 over a time period acquired in a subsequent acquisition at a specified interval from an initial acquisition of the microscopy images 20. At step 158, the methods 100, 200 further comprise identifying at least one microscopy image 22 from the subsequent acquisition acquired at the first time point and the second time point. At step 160, the methods 100, 200 further comprise producing a sequence of the identified microscopy images from the subsequent acquisition, thereby imaging the biological tissue 40 over the time period. The methods 100, 200 then comprises at step 162 determining the presence of at least one object in one or more of the identified microscopy images 22 based on one or more characteristics and at step 164 monitoring dynamic behaviour of the at least one object in one or more of the identified microscopy images 22 over the time period.

The subsequent acquisition has a role in being able to compare and analyse cell behaviour after a specified interval of time. This advantageously enables longitudinal evaluation of the same cell or location in the focal plane (x, y) assumed to contain the same cells over time. No studies have ever been performed where individual immune cells have been tracked over time after an experimental intervention in a subject's in vivo tissue, whether human or animal.

Highly motile T cells can be analysed collectively before and after an intervention, cross-sectionally between different patient groups, or individually within a short-term time lapse sequence. However, due to their rapid migratory behaviours, it is not possible to ensure re-analysis of the same cells after a certain period of time has elapsed (i.e., greater than approximately 40 minutes) as they have likely moved beyond the field of view. Over short period (minutes), it is assumed that the same cells are able to be tracked, but beyond that initial time frame cells cannot be matched with certainty.

Stromal macrophages and epithelial dendritic cells are relatively stationary; they either stay in one location and exhibit dendrite or cytoplasmic probing, or move very slowly. Because of these relative sessile behaviours, these cells can be “matched”, or analysed at different times throughout the day (ranging from about 30 minutes to up to about 24 hours, or more preferably, from about 1 hour to up to about 4 hours). The ability to compare behaviour of the same cells (or assumed to be the same cells) before and after an intervention is desirable in order to increase the precision of the effect of an intervention. An “intervention” is used herein to mean any exogenous agent that could be applied, either to the eye or more generally to the subject's body, to affect the cells.

FIG. 27 is a flow chart showing additional steps in the methods 100, 200 of FIG. 26 relating to identifying a condition or disease state based on a change in immune status of the biological tissue 40, according to some embodiments of the disclosure.

The “matched cell” approach may be used to evaluate any factor that might modify the subject's eye over a few hours. For example, a change in local environment, application of a drug/device to the eye or systemically, immunostimulatory agent such as a hyperosomotic eye drop, a surgical procedure, a vaccine, an injury or a pathogen, etc., to name a few.

The methods 100, 200 may further comprise the step 166 of comparing the monitored dynamic behaviour of the at least one object in the subsequent acquisition from the initial acquisition. The step 168 may then be performed to determine a pattern of change in the monitored dynamic behaviour for the at least one object. The pattern of change may be associated with a change in immune status of the biological tissue. Certain patterns or signatures of change (for example, the cells becoming smaller/larger) might also indicate for certain changes in the immune status of the tissue (e.g., a reduction/increase in inflammation).

In some embodiments, the methods 100, 200 further comprises the step 170 of identifying a condition or disease state based on the change in immune status of the biological tissue. The condition or disease state may be selected from at least one of a group comprising: acute or chronic inflammation of the biological tissue; an ocular disease or condition; a systemic disease or condition; and allergic conditions.

### Evaluation of Intervention

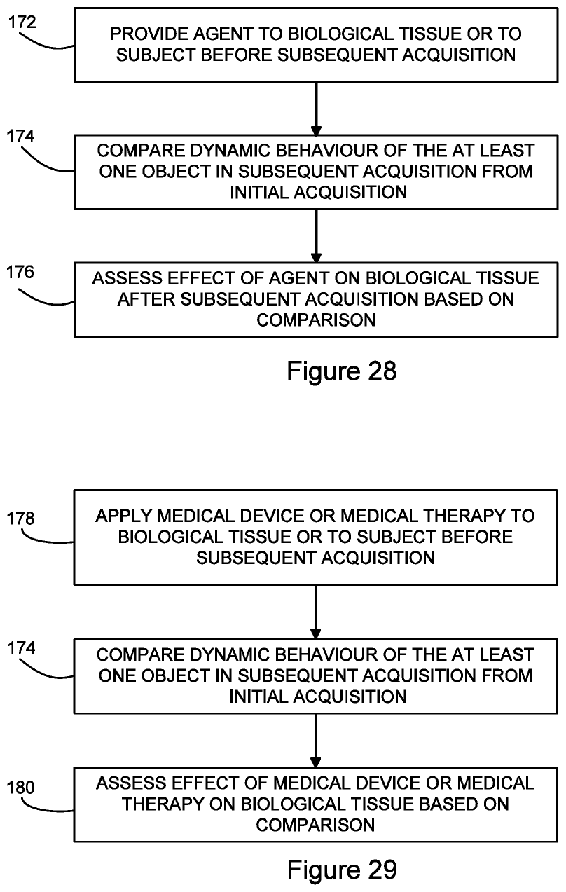

FIG. 28 is a flow chart showing additional steps in the methods 100, 200 of FIGS. 26 and 27 relating to assessing an effect of an agent on the biological tissue 40, according to some embodiments of the disclosure.

The methods 100, 200 may further comprise the step 172 of providing an agent to the biological tissue 40 or to a subject 30 having the biological tissue 40 before the subsequent acquisition. The agent may be selected from at least one of a group comprising: an environmental agent; an exogenous agent; a neurostimulatory agent; and a therapeutic agent. The therapeutic agent may be a vaccine, eye drop, or an immunomodulator (e.g., an anti-inflammatory drug). The therapeutic agent may be a topical immune agent such as a topical preservative. In other embodiments, the agent need not be a topical agent. The agent may be a nerve stimulatory agent, such as a menthol-containing product, or capsicum spray. The environmental agent may include light, smoke or pollutant exposure. The exogenous agent may include e.g., eye rubbing or exercise. The agent may be a hyperosmotic or a hypoosmotic stimulus.

The methods 100, 200 may further comprise the step 174 of comparing the monitored dynamic behaviour of the at least one object in the subsequent acquisition from the initial acquisition. Equally, static behaviour may be monitored in the sense of a static feature such as morphology of cells, density of cells and the like—which are static but can be tracked over time. It will be appreciated that dynamic behaviour refers to motility (i.e. how much and in what way do they move for example). The methods 100, 200 may further comprise the step 176 of assessing an effect of the agent on the biological tissue 40 after the subsequent acquisition based on the comparison of the dynamic behaviour of the at least one object from the initial acquisition.

FIG. 29 is a flow chart showing additional steps in the methods 100, 200 of FIGS. 26 and 27 relating to assessing an effect of a medical device or medical therapy on the biological tissue 40, according to some embodiments of the disclosure.

The methods 100, 200 may further comprise the step 178 of applying a medical device or medical therapy to the biological tissue 40 or to a subject 30 having the biological tissue 40 before the subsequent acquisition. The medical therapy may be an ocular therapy. The medical device may be a contact lens. The contact lens may be one or more of: a drug-eluting contact lens or bandage contact lens. In other embodiments, the medical device may be an amniotic membrane or a fluid gel. The medical device may include artificial tear products or a gonioscope. The medical therapy may include light therapy, such as intense pulse light therapy which may be applied to skin around the subject's face to affect the biological tissue 40. In other embodiments, the medical therapy may include thermal or pressure therapy, such as LipiFlow® therapy (which may be applied to the eyelid glands to affect the biological tissue 40.

The methods 100, 200 may further repeat the step 174 of comparing the dynamic behaviour of the at least one object in the subsequent acquisition from the initial acquisition. The methods 100, 200 may further comprise the step 180 of assessing an effect of the medical device or medical therapy on the biological tissue 40 after the subsequent acquisition based on the comparison of the dynamic behaviour of the at least one object from the initial acquisition.

### Computer-Implemented IVCM Techniques

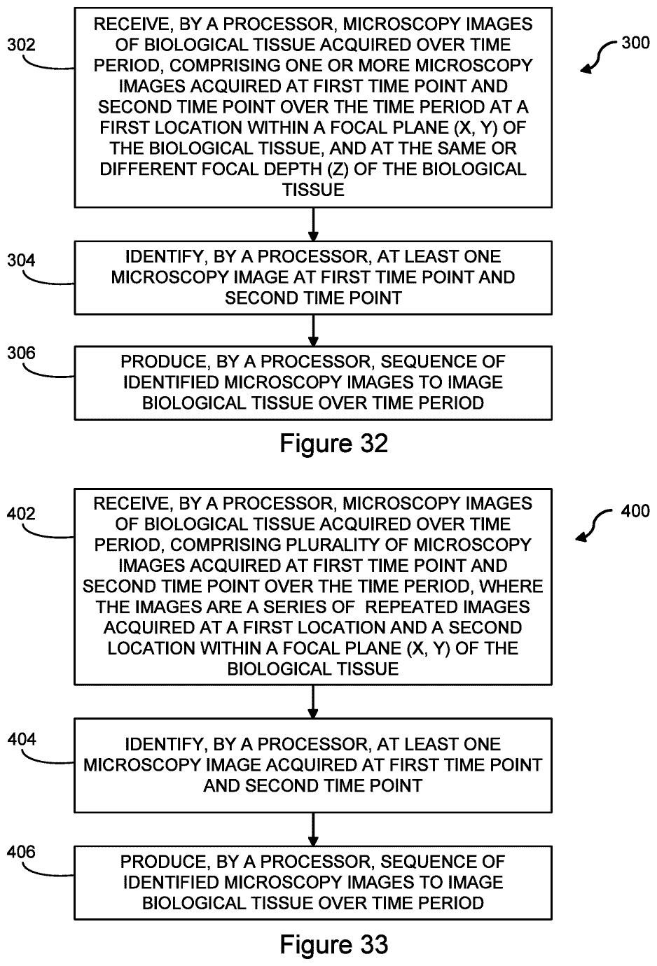

FIG. 32 is a flow chart illustrating a computer-implemented method 300 for imaging a biological tissue 40 over time where each of the steps are performed by a processor 504 (see FIG. 34), according to some embodiments of the disclosure. The method 300 comprises the step 302 of receiving, by a processor 504, microscopy images 20 of the biological tissue 40 acquired over a time period, comprising: one or more microscopy images 20 acquired at a first time point over the time period, wherein the one or more microscopy images 20 are acquired at a first location 50 within a focal plane (x, y) of the biological tissue 40 and at a focal depth (z) of the biological tissue 40; and one or more microscopy images 20 acquired at a second time point over the time period, wherein the one or more microscopy images 20 are acquired at the first location 50 within the focal plane (x, y) of the biological tissue 40 and at the same or a different focal depth (z) of the biological tissue 40. The method 300 also comprises the step 304 of identifying, by a processor 504, at least one microscopy image 22 acquired at the first time point and the second time point. The method 300 also comprises the step 306 of producing, by a processor 504, a sequence of the identified microscopy images 22, thereby imaging the biological tissue 40 over the time period. In some embodiments, the method 300 further comprises the step of determining, by a processor 504, the presence of at least one cell in one or more of the identified images 22 based on one or more characteristics. The method 300 may also comprise the step of monitoring, by a processor 504, dynamic behaviour of the at least one cell in one or more of the identified images 22 over the time period.

FIG. 33 is a flow chart illustrating another computer-implemented method 400 for imaging a biological tissue 40 over time where each of the steps are performed by a processor 504 (see FIG. 34), according to some embodiments of the disclosure. The method 400 comprises the step 402 of receiving, by a processor 504, microscopy images 20 of the biological tissue 40 over a time period, comprising a plurality of microscopy images 20 acquired at a first time point over the time period, wherein the plurality of microscopy images 20 is a series of repeated images acquired at a first location 50 and at a second location 52 within a focal plane (x, y) of the biological tissue 40; and a plurality of microscopy images 20 acquired at a second time point over the time period, wherein the plurality of microscopy images 20 is a series of repeated images acquired at the first location 50 and at the second location 52 within the focal plane (x, y) of the biological tissue 40. The method 400 also comprises the step 404 of identifying, by a processor 504, at least one microscopy image 22 for the first time point and the second time point. The method 400 also comprises the step 406 of producing, by a processor 504, a sequence of the identified microscopy images, thereby imaging the biological tissue over the time period. In some embodiments, the method 400 further comprises the step of determining, by a processor 504, the presence of at least one cell in one or more of the identified microscopy images 22 based on one or more characteristics at the two or more locations 50, 52. The method 400 may also comprise the step of monitoring, by a processor 504, dynamic behaviour of the at least one cell in one or more of the identified microscopy images 22 at the two or more locations 50, 52 over the time period.

The computer-implemented methods 300, 400 may also include any one of the steps of embodiments of the methods 100, 200, respectively, as described above being implemented by a processor 504. Thus, the methods 300, 400 may be entirely computer-implemented by means of receiving microscopy images from a microscope 10, and involve software processing of the image data to determine the presence of a cell(s) and monitor dynamic behaviour of the cell(s) over the time period.

FIG. 34 is a schematic illustrating a system 500 for imaging a biological tissue 50 over time, according to some embodiments of the disclosure. The system 500 comprises a communication interface 502 for receiving microscopy images 20, a processor 504 and a computer-readable medium (e.g., memory device 506) which is communicatively coupled to the processor 504. The computer-readable medium stores computer-executable instructions that when executed by the processor 504, causes the processor to perform the steps of the methods 300, 400 set out in the embodiments above with reference to FIGS. 32 and 33.

The microscopy images 20 are received by the processor 504 for imaging the biological tissue. The microscopy images 20 may be acquired by a microscope 10 as described with reference to FIGS. 1 to 8 and the methods 100, 200 of embodiments of the disclosure. The microscopy images 20 may be received by the processor via the communication interface 502. The communication interface 502 may be include an electronic circuit for enabling telecommunication with the microscope 10 or microscopy system. The communication interface 502 may communicate via a hard-wired or wireless connection with the microscope 10. For example, the communication interface may be a wireless connection such as Bluetooth. The microscopy images 20 may then be communicate through an electronic circuit or coupling with the processor 504.

The processor 504 may be any known Computer Processing Unit (CPU) as would be appreciated by a person skilled in the art. For example, the processor 504 may include a Celeron chip by Intel Corporation located, for example, on an ETX form factor PCB, or a controller, such as a microcontroller. The processor 504 may be adapted to be in communication with the computer-readable medium, such as memory device 506, which may include a memory device having a non-volatile memory, such as a hard drive. Additionally/alternatively, the memory device 506 may provide for temporary storage of instructions obtained wirelessly, such as updated by a Cloud-based server. The processor 504 may be adapted to execute instructions stored in the memory device 506 for performing steps in the methods 300, 400.

In some embodiments, the system 500 may optionally include an output device 508 with a display 510 as shown in FIG. 34. The output device may be Visual Display Unit (VDU), such as a monitor of a computer. These elements may enable graphical display of (i) the sequence of the identified microscopy images forming the time-lapsed video, and (ii) the sequence of the identified microscopy images forming the combined colour image of overlaid images forming a 2D representation. The display of this information may then be used by an operator to interpret changes in object behaviour (e.g., cell behaviour) over the time period.

It is to be understood that various modifications, additions and/or alternatives may be made to the parts previously described without departing from the ambit of the present disclosure as defined in the provisional claims appended hereto.

### Identification of a Condition or Disease State in a Subject

The present disclosure relates to methods of identification, evaluation or diagnosis of the presence of or a pre-disposition to a condition or disease state in a subject, for example, chronic or acute inflammation of the eye. The methods comprise determining the morphology, or number, or density, or dynamic behaviour of at least one immune cell at one or more locations within a focal plane (x, y) and/or at one or more focal depths (z) of the biological tissue (e.g., the cornea) of the subject and comparing the level to a reference or baseline level.

The term “reference level” refers to the morphology, or number, or density, or dynamic behaviour of a corresponding cell type at one or more locations within a focal plane (x, y) and/or at one or more focal depths (z) of the biological tissue of a normal control subject(s) that does not have the condition or disease state, or in a subject(s) with the disease or condition but treated with a therapeutic agent/therapy (e.g., ocular therapy such as immunomodulatory treatment), or in an unstimulated subject including baseline level of the subject prior to exposure to an agent (e.g., an environmental agent, an exogenous agent, a neurostimulatory agent, a dietary supplement (e.g., fish oil), or a therapeutic agent). The reference level may be a threshold level, for example, a cut-off value.

The methods may require determining a relative difference of the morphology, or number, or density, or dynamic behaviour of at least one immune cell at one or more locations within a focal plane (x, y) and/or at one or more focal depths (z) of the biological tissue (e.g., the cornea) of the subject compared to the reference level or baseline level; or alternatively determining an absolute measurement (e.g., absolute density of the immune cell) that can be compared, to for example, a diagnostic or prognostic cut off threshold.

“Evaluation” in the context of the present disclosure relates to the assessment of the severity of the disease or condition in a subject.

“Diagnosis” in the context of the present disclosure relates to the recognition and (early) detection of a disease or condition in a subject and may also comprise differential diagnosis and pre-disposition of the subject to develop the disease or clinical condition. A diagnosis may include an assessment of the degree of disease/condition severity (e.g., “low” to “high”), current state of disease progression (e.g., “early”, “middle,” or “late” stages), or include a comparative assessment to an earlier diagnosis (e.g., “advancing”, “stable”, or in “remission”).

“Prognosis” denotes a prediction of how a subject's (e.g., a patient's) disease or condition will progress. This may include a prediction of the chance of recovery or the chance of an adverse outcome for said subject. Prognosis may also include a prediction of disease/condition response to treatment.

The term “correlating” or “correlated” as used herein refers to comparing the morphology, or number, or density, or dynamic behaviour of the at least one immune cell at one or more locations within a focal plane (x, y) and/or at one or more focal depths (z) of the biological tissue of the subject over time to its amount in subjects known to have, or known to be at risk of, a given disease or condition, or subjects known to respond or not respond to a given treatment.

The morphology, or number, or density, or dynamic behaviour of the at least one immune cell at one or more locations within a focal plane (x, y) and/or at one or more focal depths (z) of the biological tissue of the subject can be compared to a level known to be indicative of an abnormal state. In some embodiments, the level has been correlated with a diagnosis; that is, the skilled person can use the level to determine whether the subject has a disease or condition or a pre-disposition thereto and respond accordingly. Alternatively, the level can be compared to a level indicative of a normal state. In some embodiments, the level has been correlated with the absence of disease or a condition. In other embodiments, the level can be compared to a baseline level of the morphology, or number, or density, or dynamic behaviour of the at least one immune cell at one or more locations within a focal plane (x, y) and/or at one or more focal depths (z) of the biological tissue of the subject taken from the subject at an earlier timepoint, to, for example, monitor disease progression or treatment efficacy. In some embodiments, the level can be compared to a baseline level taken before initiation of treatment or following a round of treatment. In other embodiments, the level has been correlated with a response or non-response to a given treatment.

In some embodiments of the disclosure, the morphology, or number, or density, or dynamic behaviour of the at least one immune cell at one or more locations within a focal plane (x, y) and/or at one or more focal depths (z) of the biological tissue of the subject can be used to evaluate the severity of the disease or condition. For example, the morphology, or number, or density, or dynamic behaviour of the at least one immune cells at one or more locations within a focal plane (x, y) and/or at one or more focal depths (z) of the corneal tissue of the subject can be used to evaluate the severity of chronic or acute inflammation of the eye. The subject can be stratified by risk and/or treatment.

The term “risk stratification” in the context of the present disclosure refers to a probability of specified outcomes for a subject.

The term “treatment stratification” in the context of the present disclosure refers to the choice and/or adjustment of a therapeutic treatment of the subject.

Imaging of biological tissue via a scanning instrument may take a number of forms including in vivo confocal microscopy (IVCM) where imaging of the cornea of the eye is carried out with a confocal scanning laser ophthalmoscope or the like. However, acquiring useful imaging data can prove challenging and, once acquired, images typically suffer from noise, artefact, and a limited field of view, due to the natural motion of the eye when the images are being captured. These factors limit the degree to which the same cells can be identified and tracked over time. The present invention provides a system and method for image processing on a scanning instrument to improve noise, distortion and provide a series of images that can be used to produce a time-lapsed video. Advantageously, this system and method therefore enable tracking of immune cell migration and movement, over a short period of time (for example, minutes).

The system and method for image processing automatically registers and processes the acquired images to significantly improve contrast, resolution and field of view. The system and method for image processing further, automatically montages scans of the same tissue region, allowing cell dynamics (e.g., motility, dendrite probing) to be accurately tracked. Advantageously, minimal user intervention is required. It will be appreciated that the system and method for image processing may be applied to any number of scanning instruments or measurements of biological tissue—where there is movement or the like.

In an embodiment, the system and method for image processing may be employed to cross-sectionally and longitudinally assess the dynamics of immune cells of the corneal epithelium and stroma, using the time-lapsed in vivo confocal microscopy imaging technique disclosed herein. It will be appreciated that the system and method for image processing is also well suited to other in vivo cellular imaging modalities, for example adaptive optics scanning laser ophthalmoscopy (AOSLO), scanning laser ophthalmoscopy (SLO), optical coherence tomography (OCT), or other scanning-based imaging applied to moving tissue (for example, the eye).

Advantageously, the system and method for image processing can substantially improve the efficiency and consistency of time-lapsed video creation from static IVCM images, to enable the collection of dynamic data for clinical and research applications, across various imaging for studying the eye with cellular resolution.

A problem with data acquisition and processing from scanning instruments is that they have bottlenecks which slow down workflow. For example, in corneal IVCM, the individual image (or frames, a plurality of which may make up a video or time-lapse) captured within an IVCM “video” of sequentially acquired images are noisy and, in current commercial implementations, can suffer from non-uniformity of illumination across the imaging field. A second problem is repeated and random motion of the eye (i.e., microsaccades) that inherently occur during acquisition (typically over several seconds) both shift the area of tissue imaged and create a unique pattern of intra-image distortion within each acquired image. Attempts have been made to address this by registering rectangular strips of tissue that include the entire “fast” dimension of the image (swept at kilohertz rates by a resonant scanner), but only a portion of the “slow” dimension (prone to distortion due to intra-scan eye movements). One image (or frame, a plurality of which may make up a video or time-lapse) is typically selected as a “key frame”, distorted by an unknown amount, to which all other images are aligned. Despite embedding distortion into the acquired video, this approach enables the averaging of noisy video data to create a high signal-to-noise averaged image from each acquired video.

Another problem is that the alignment of images acquired at different time points may be intractable, due to torsional misalignment resulting from head rotation between scans. In this case, strip registration is unsatisfactory as there is no shared rigid image dimension between time points if the images are rotated. Another issue is that the motion of cells over time directly interferes with image registration process. This suggests the need for a filtering operation to “lock on” to stable tissue landmarks.

Finally, the appearance of the corneal tissue architecture changes markedly through its depth; for example, the epithelium contains branching linear structures (nerves), compared with the stroma that features a distinct cellular mosaic (keratocytes). Ideally, image registration software would operate equally well, regardless of the structural features of the tissue imaged.

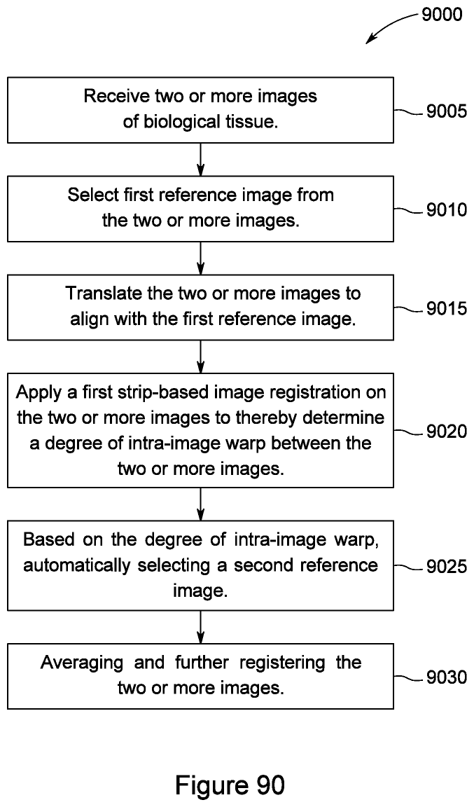

FIG. 90 is a flow diagram 9000 illustrating an image processing method for a scanning instrument for scanning biological tissue—and for convenience will be described with reference to corneal IVCM but it will be appreciated that it may apply to any other application where a scanning instrument is used with biological tissue where movement is present during capture.

In an embodiment, the system and method for image processing allows for enface intravital imaging acquisition and subsequent processing, to generate high-quality, time-lapsed videos of the dynamics of immune cells (and other motile features as will be appreciated) in the corneas of living humans.

The system and method for image processing yields a view of stabilized background tissue, superimposed with dynamic motion of immune cell subsets (i.e., T cells, dendritic cells and macrophages), revealing the dynamic behaviours. The confocal imaging device used to acquire the raw images may be a commercial instrument commonly used in clinical and research settings, and the processing of images is largely entirely automated.

The system and method for image processing provides an averaged (or montaged) image from each acquired series of images (forming a video or time-lapsed video) having a wider overall field of view compared with that of a single image or frame, as a consequence of the unavoidable motion of the eye during the several-second image capture period shifting the imaged field to reveal adjacent tissue regions.

Tissue outside the nominal reference image or frame is often ignored in strip registration as it is non-trivial to decide how to include potentially distorted data from other images or frames. This issue is negligible in the fast (horizontal) direction due to the fast row acquisition speed. However, in the vertical or “slow” direction, some care must be taken. Extrapolation may be possible and undertaken where intra-strip warp is minimal, allowing strips to be confidently placed such that they “hang off” the reference image in the vertical direction.

In an embodiment, the system and method for image processing may facilitate extrapolation in a controlled manner by: (i) automatically selecting a low-distortion reference image; (ii) updating the reference image during registration of a video to create a running average; (iii) registration of images in an ordered sequence according to their similarity to the running average which generally minimized intra-strip warp such that extrapolation in the vertical direction introduced little distortion; (iv) outlier rejection of strip shifts, which act to exclude rapid eye movements seen in an otherwise steady image; (v) the requirement of at least 50% overlap in the area of registered tissue for each strip; (vi) extrapolation only being permitted where there was not already existing tissue in the reference image; and (vii) a final iteration of strip registration using the averaged image from the penultimate step. Advantageously, these steps allow improvement to the overall field of view, and include data from most images (or frames, a plurality of which may make up a video or time-lapse).

Existing approaches when composing high-quality cellular images of the retina tend to incorporate only a subset of images or frames, chosen to maximize the signal-to-noise ratio by mitigating the influence of variations in tear film, accommodation, eye movements, dynamic aberration correction, irregularities in the scanning hardware, etc. This is sometimes referred to as “lucky averaging”. In contrast, the system and method for image processing of the present invention does not use this approach since contact-based imaging of the anterior eye is resilient to many of the factors described above. The system and method for image processing present invention may utilise a multi-stage registration procedure which results in the ability to include most of the image/frame information without further inclusion criteria, beyond the rejection of poor strip registrations.

As will be appreciated, the processing time required for image registration for the system and method for image processing of the present invention may exceed most comparable approaches (for example, requiring approx. 40-60 minutes to register six sequence scan videos and then co-registering them to produce a time-lapsed video). In an embodiment, less rounds of strip registration may be used, or the software may be further optimised to improve processing speed (for example, via parallelization or the elimination of redundant calculations and the like). However, the rate-limiting step in strip registration approaches is typically manual user input to decide on an appropriate minimally-distorted reference image or frame.

The method starts at step 9005 where two or more images of biological tissue (for example, corneal tissue) are received. It will be appreciated that more than two images may be provided, for example 100 images may be provided—these images may then ultimately be combined to form a video or time-lapsed video.

Control then moves to step 9010 where a first reference image is selected from the two or more images. Each of the images may be automatically inspected by an image processor (such as ImageJ or the like), where for example, obvious sources of error, such as large eye movements or a change in focus to a different tissue layer (for example, if the participant moved axially during the acquisition process) are noted. The corresponding images may then be removed (so as not to form part of the final output—which may be a series of images ultimately combined to form a video or time-lapsed video).

As an example, where 100 images are used and corresponding images are then removed, this may result in the order of 80 of 100 unique images remaining (for ease of reference, 80 to 100 images may equate a video or time-lapsed video—which in the order of 7 seconds duration. The inventors have found that if there are fewer than about 50 images, the output video should be discarded as the selection of an appropriate reference image or frame requires a sufficient number of images or frames.

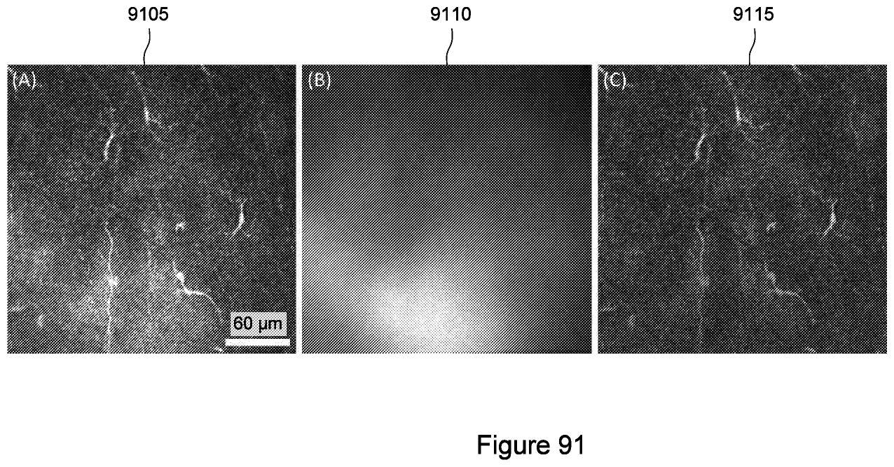

In an embodiment, flat-fielding of individual video image frames may be utilised for corneal data, but depending on the application, may not be required, particularly if the scanning instrument is well calibrated. Raw image data acquired from scanning instruments (such as a commercial IVCM device) can be subject to non-uniformity of image intensity across the field (i.e., tissue brightness may vary depending on location within the image). Such bias can occur due to differences in dwell time of the scanning instrument across their scan path, or due to focusing errors whereby the spot illuminated on the tissue is not imaged sharply onto the plane of the confocal pinhole. Advantageously the image processing system and method of the present invention corrects for this phenomenon by taking advantage of the natural motion of the eye across acquired images (for example 100 images which may resulting in an output of a time-lapsed video in the order of 7 seconds). By averaging each unregistered video pixel over time, structural information pertaining to the tissue itself is averaged away, which leaves apparent only the systematic intensity bias. This average is normalized and used to create a “flat field” image by which all images (or frames, a plurality of which may make up a video or time-lapse) are divided to rectify the recorded intensity of each pixel. In the field of retinal imaging with another scanning instrument such as AOSLO, such approaches are not typically required as great effort is taken to correct for the potential sources of error outlined above. FIG. 91A shows an example raw image or frame 9105, which is acquired at the level of the human corneal basal epithelium using IVCM and stretched to fill a grayscale colour map. It should be noted that there are many saturated pixels that are not handled specifically (i.e., removed) in any of the processing steps below. FIG. 91B shows the flat field image 9110 which in this case was computed as the average of 100 unregistered images/frames and FIG. 91C shows the raw image/frame 9115 after the flat-fielding operation applied to the image 9105.

Control then moves to step 9015 where the two or more images are translated to align with the first reference image before a first strip-based image registration is applied at step 9020 to the two or more images to thereby determine a degree of intra-image warp between the two or more images.

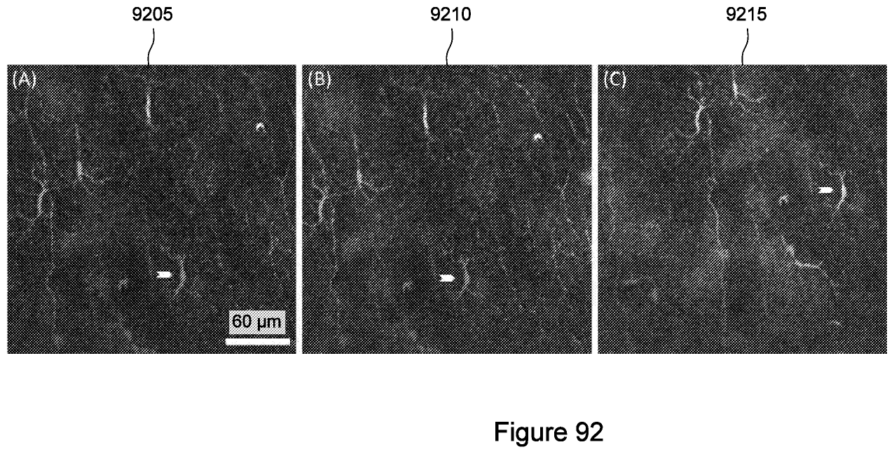

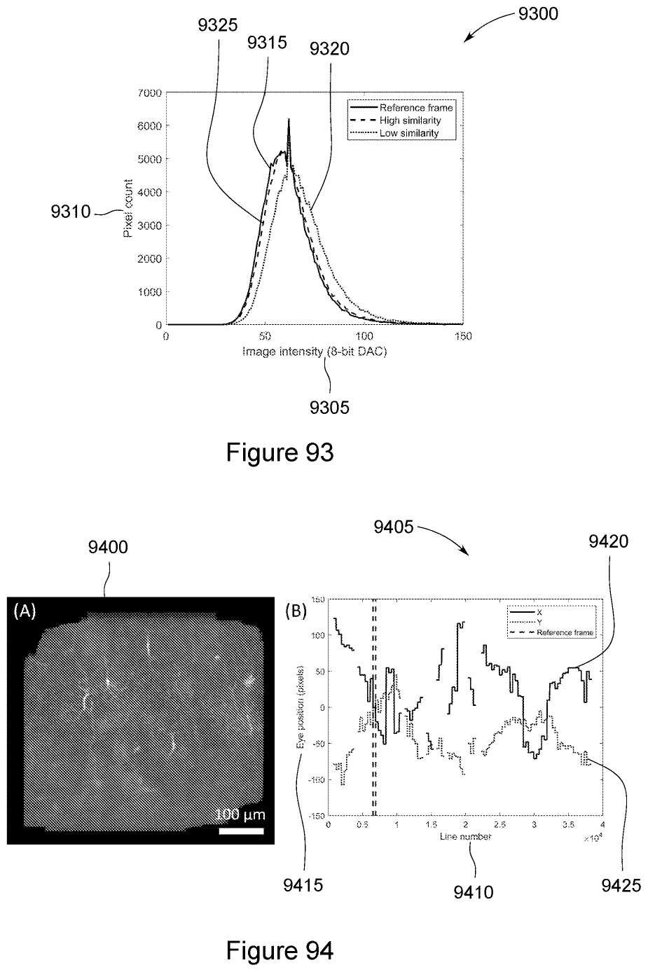

The first strip-based image registration requires that two or more images are brought into coarse alignment. To ensure a desirable number of successfully registered images, it is preferable to maximize the overlap between each image to be registered and the set of images that have already been registered. To achieve this, a pairwise histogram similarity between all images may be determined. Similar image histograms indicate that a similar region of tissue has been imaged. The image with the greatest overall similarity to the rest of the images is selected as the first reference image to be registered. Other images are then registered to the first reference image, in order of their own similarity to that image. Representative image histograms are shown in FIG. 93, and their corresponding images, are shown in FIG. 92.

An example reference image or frame 9205 is shown in FIG. 92A; another image or frame 9210 showing an almost identical tissue region is shown in FIG. 92B for which the relative distances between cellular and sub-cellular processes differs—that is, the image is distorted (in fact, both images are distorted, in different ways, due to the unique pattern of eye movements occurring during the acquisition of each image or frame). In addition, another image or frame 9215 showing a largely non-overlapping piece of tissue is shown in FIG. 92C.

The image histograms for each image or frame from FIG. 92A, FIG. 92B and FIG. 92C are plotted on chart 9300 in FIG. 93—a chart of image intensity 9305 vs pixel count 9410; where images with aligned tissue regions have similar image histograms, despite their relative distortion (which should, in principle, have minimal influence on the histogram except for where the tissue is shifted outside the image). Plot 9315 (blue) and plot 9325 (red) correspond to FIG. 92A and FIG. 92B respectively, showing high similarity, whereas plot 9320 (yellow) shows a slightly different histogram shape and includes less overlapping tissue, corresponding to FIG. 92C.

Fourier-based normalized cross-correlation may be used to correct for image translations. The two or more images may be first de-trended to remove coarse image gradients. Since the normalized cross-correlation can return spuriously high values towards the edges of images, due to noise; a threshold may be set such that only cross-correlation peaks corresponding to more than 50% overlap in area are considered (equivalent to 25% linear overlap). The remaining location of highest cross-correlation may then be taken, with the displacement of this point from the image centre directly giving the horizontal and vertical image shift. Since there is unlikely to be significant rotation of acquired images, there is no need to correct for ocular torsion—but depending on the application—correction for ocular torsion may be required.

It will be appreciated that each successfully registered image or frame may be added to a running average which serves as the new reference image for registration of the next image in a sequence, until all images are registered. In an embodiment, registered sequences of images may be cropped to minimize the number of non-illuminated pixels, reducing overall file size and memory requirements.

In an embodiment, the images are padded out by a suitable number of pixels such that the image data does not get shifted out of the frame/image. As will be appreciated, the amount of padding on the pixels will depend on the images and the application—but, for example, may be in the order of 100 pixels. Padding of pixels is to ensure that the dynamic motion of the eye does not shift the registered images tissue out of the array, which would limit the size of the acquired field. Translations reaching the image border triggered padding of the array by an additional 100 pixels, and the registration for that image may be repeated. If additional padding is indicated on the repeat (i.e. a shift of 200 pixels relative to the reference), the image or frame may be discarded. Advantageously, this approach avoids the need to estimate the maximum eye motion across a scan, and the use of needlessly large image arrays that would slow computation time and increase memory requirements.

It will be appreciated that images may also be discarded if the correlation between the first reference image and registered images is lower after, compared with prior to, registration, or if the correlation is considered to be an “outlier” compared with all correlations—for example being more than three scaled median absolute deviations (MADs) from the median.

FIG. 94A is an image 9400 showing the average intensity for full-field translations within a single 100 image video. This is the average output after full-field registration of 100 images to the first reference image that was shown in FIG. 92A. The influence of intra-image distortion can be observed, with blurring in the vertical direction, away from the center of the registered image (i.e., in the direction of the slow scanner). FIG. 94B is a chart 9405 of line number 9410 vs Eye position (pixels) 9415 having plots 9420, 9425 showing the inferred change in position of the tissue due to eye movements over the course of the video sequence acquisition. The square nature of the plot indicates that information regarding intra-image eye motion is not yet known. The first reference image (dashed vertical lines) lies generally in the center of the distribution of eye positions, which was both the intended and typical result of the histogram similarity described above. Missing data indicates registrations that were rejected.

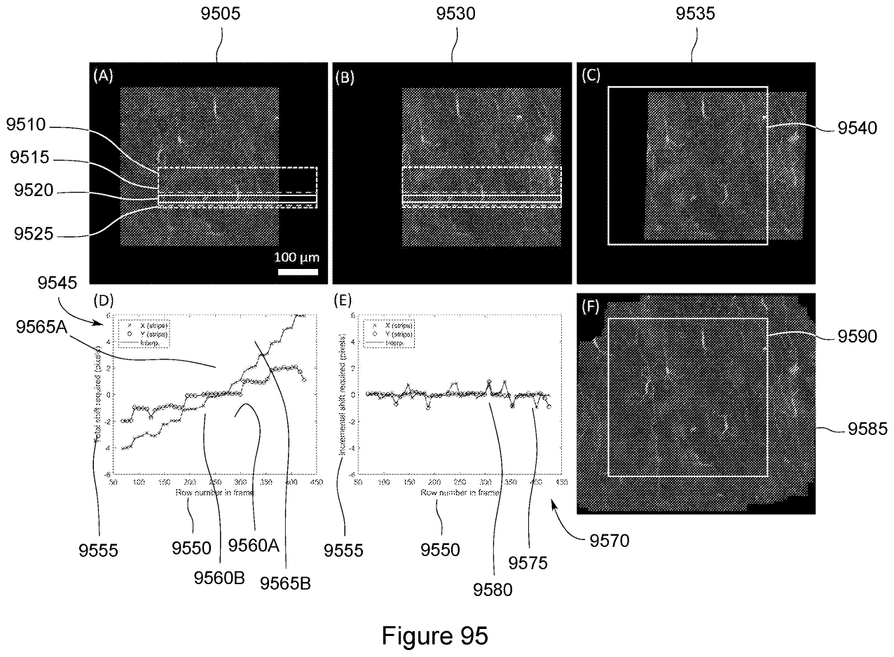

The detailed operation of first strip-based image registration at step 9020 involves the selection of an appropriate first reference image, and the partitioning of each image to be registered into rectangular strips, each of which are registered to the first reference image. In an embodiment, further strip registration may be applied (i.e. in a series of stages). However, in first strip-based image (a first stage), the reference image is chosen as the image with greatest histogram similarity to the average full-frame registered image from step 9010 (i.e., the single image most like the average shown in FIG. 94A; the most similar such frame is shown as 9505 in FIG. 95A).

First strip-based image registration may be carried out in descending order of similarity to this new reference image frame. FIG. 95B shows an image 9530 of an example ‘other’ image to be strip-registered, and FIG. 95C shows an image 9535 of the outcome of the registration. The green border 9540 indicates the extent of the reference tissue image (as shown in FIG. 95A).

For a given image, first strip-based image registration is preferably applied three times (not to be confused with the three stages referred to above, of which this is the first). In the first iteration, the size of the strips 9510 may be one quarter that of the image height. With a typical image height of 384 pixels, this corresponds to a strip height of 96 pixels; a representative example is shown by the red boxes 9515 in FIG. 95A and FIG. 95B. Strip heights may be 32 pixels in the second iteration (yellow boxes 9520), and 16 pixels in the final iteration (blue boxes 9525). The estimated shift for each strip is taken to correspond to the central row of that strip, with linear interpolation used to recover a displacement map applicable to all rows in the image.

FIG. 95D is a chart 9545 of row number in image 9550 vs shift required (pixels) 9555 which shows the strip estimates (crosses 9560A, 9565A) and interpolated row estimates (lines 9560B, 9560B) for the example image or frame shown in FIG. 95B, in the third and final iteration of strip registration. Each iteration is seeded by the displacements determined from the previous iteration, such that images are better aligned with each subsequent iteration. FIG. 95E is a chart 9570 of row number in image 9550 vs incremental shift required (pixels) 9555 which plots the incremental shifts required in the final iteration for this example showing strip estimates (crosses 9560A, 9565A) and row estimates (lines 9560B, 9560B).

Regardless of the strip height, strips tile the image in a redundant fashion such that the beginning of each strip is half of one strip height from the beginning of the preceding strip (i.e., in the third pass the strips are separated by 8 pixels vertically). Each strip is registered to the corresponding strip in the first reference image via normalized cross-correlation. In the second and third iterations, the strip location in the first reference image is updated based on the information from the preceding registration. In this way, despite the shrinking strip size, it is generally expected that subsequent registrations benefit from a higher degree of strip overlap. Accordingly, if the identified shift, relative to the expected position of the strip, is more than half the strip height (vertically) or more than one quarter the image width (horizontally), the registration may be discarded.

An outlier rejection procedure may be applied at the level of whole images, strips with measured shifts that are outliers relative to the rest of an image may also be also discarded.

The raw eye movement information (sampled at intervals of 8 to 48 pixels, as described above) may further be interpolated to produce a continuous estimate of image movement, horizontally and vertically, across the full image or frame. A cubic spline interpolant may be used for the first two iterations, with linear interpolation used for the final pass. Example raw and interpolated eye movement information is plotted in FIG. 95D.

On the final (third) iteration (strips 16 pixels high), strip shifts that are more than 3 pixels from the expected position may be discarded, as these correspond to significant intra-strip warping and hence to excessive eye movements during the scanned acquisition.

To apply the measured strip registration, the interpolated values above are used to populate a 2D deformation map. Missing data in this map (for example, due to discarded strips or due to the strip tiling not matching the extent of the image) may be populated with the value from the nearest valid row (i.e., the nearest valid point in time). Advantageously, this maximizes the registered field of view as it avoids discarding tissue that was not present in the original first reference image. The effect of this can be seen in FIG. 95C, where imaged tissue has been placed in the registered image that was not visible in the first reference image (green border 9540); the additional tissue region is positioned in the registered array to not induce any discontinuity with the other portions of the image or frame that did overlap. This approach could potentially introduce distorted reference image information to the registration procedure in the case that eye movements change suddenly in the non-common portion of the image to be registered. However, the inventors have found that: (i) the ordering of images or frames to be registered according to their similarity with the reference reduces the chances that the image edge is highly distorted relative to the rest of the image, and (ii) the reference image is, itself, distorted to some degree.

The 2D deformation maps in the horizontal and vertical dimensions, resulting from the procedure described above, are used as input to a 2D interpolation procedure with bilinear interpolation to carry out the image warps required.

The outcome of the first stage of strip registration (i.e., first, second and third iteration steps) for the whole example video image sequence is shown by the averaged image 9585 shown in FIG. 95F. The location of the reference image or frame is indicated by the green box 9590, showing the utility of extrapolating outside the bounds of the reference tissue, as described above, for increasing the size of the final image field of view.

At step 9025, based on the degree of intra-image warp determined at step 9020, the method automatically then selects a second reference image. The second reference image is an ideal reference image.

Having completed the first strip-based image registration step 9020, the registered imagery (e.g., FIG. 95C and FIG. 95F) may be discarded. The purpose of the first strip-based image registration is to use the registration information (i.e., FIG. 95D) to then automatically identify and select a second (ideal) reference image. The second reference image is preferably a suitable minimally-distorted reference image, from the approximately unique 100 images that were originally acquired/received, in a preferred embodiment using 100 images.

A suitable robust heuristic may be provided to determine which image in sequence of images is minimally distorted. Essentially, the frame/image whose distortion map is most similar (least root-mean-square error) to the set of measured distortion maps in the video, is chosen. The heuristic preferably underscores the minimization of distortion, namely the stochastic nature of eye movements. Eye movements, and hence the deformation maps imparted by them, are random in the sense that no piece of tissue should expect to be deformed in a more positive or more negative manner than any other piece of tissue—that is, there should be no bias in tissue distortion. Accordingly, distortion maps corresponding to high distortion should appear dissimilar both to other high distortion maps (due to the uniqueness of the distortion produced) and to low distortion maps. Low-distortion maps should appear relatively similar to other low-distortion maps, and should, to a certain extent, straddle the distribution of positive and negative image distortions seen in the higher deformation maps. For these reasons, given a sufficiently large number of images, a minimally distorted map can be selected by comparing each of the distortion maps generated in the preceding step with each of the other distortion maps. The image or frame whose distortion map is most similar to the average distortion map may be chosen as the second reference image.

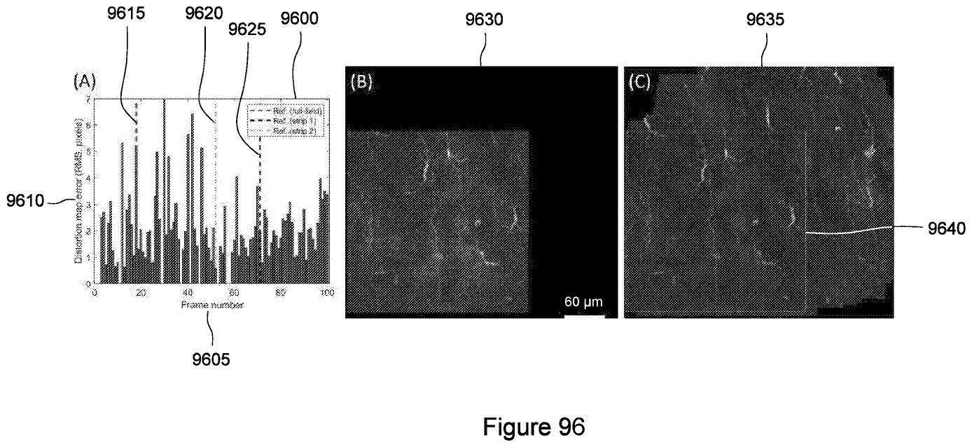

An associated error metric is plotted for the example series of images or frames (which may form a video) in chart 9600 of frame/image number 9605 vs distortion map error 9610 of FIG. 96A, where the new image to be used as the reference image (green line 9620) having a lower error value than the reference images selected at step 9010 (i.e., for full-field translation, and for the first stage of strip registration, shown as red line 9615 and black line 9625 respectively).

In an embodiment, having identified an appropriate second reference image from the heuristic, a second strip-based image registration may be applied to align the two or more images with the second reference image. Essentially step 9010 is carried out again but with the use of the second reference image. The second reference image selected at this stage for the example image 9630 is shown in FIG. 96B; the corresponding averaged strip registered image for the entire video 9635 is shown in FIG. 95C. As can be seen from the marginal position of the reference tissue (green square border 9640) relative to the rest of the registered imaged tissue, the reference image or frame here was not chosen to coincide with the average tissue location imaged in the other video image/frames.

It may be noted that image quality does not appear better at this stage (e.g., compare FIG. 96C to FIG. 96F). There is merely a rearrangement of the tissue according to the reduction of distortion. However, following the heuristic above, the image produced at this stage should be a more faithful representation of the true anatomy. Advantageously, this arrangement leads to minimal image warping being required to make comparisons between different time-points.

In a further embodiment, a third strip-based image registration may be applied to expand the field of view in the slow scan direction. In this case, strip registration is repeated an additional time, now using the averaged image from the second strip-based image registration step as the reference (i.e., FIG. 96C in the representative example). The reason for this is that in the preceding stage, extrapolation beyond the reference tissue region is limited due to suboptimal image overlap and/or incomplete tiling of strips, such that some portions of some images or frames are discarded. By using the average of the registered sequence in the preceding stage as the reference, these issues are largely ameliorated and a maximal field of view is obtained at the level of each individual image or frame, and hence for the entire video sequence. Use of the wider field averaged image as the reference also helps to mitigate poor strip registrations that can occur near the edge of the reference image.

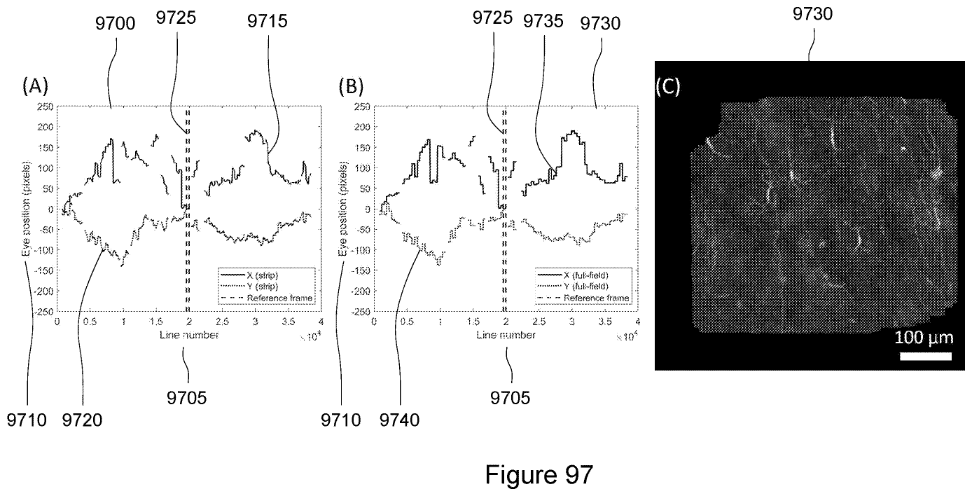

Following the first, second or third strip-based image registration, the pattern of inferred eye movements across the images may be plotted as a chart 9700 (as shown in FIG. 97A). Chart 9700 is line number 9705 vs eye position (pixels) 9710 and shows plots of X (strip) 9715 and Y (strip) 9720. Estimated eye position across the full video image acquisition, with the reference image or frame period shown by dashed lines 9725. This can be contrasted with the full-field translations, relative to the same reference, plotted in chart 9730 of FIG. 97B which is line number 9705 vs eye position (pixels) 9710 and shows plots of X (full-field) 9735 and Y (full-field) 9740 having reference frame 9725. The final averaged image across this example image sequence is shown in image 9730 FIG. 97C. The field of view is slightly reduced compared with FIG. 97C; here, pixels illuminated fewer than three times were discarded to reduce the impact of noise when attempting to track tissue across time points.

Control then moves to step 9030 where the two or more images are averaged and further registered.

While steps 9000 to 9025 provide a series of high-quality images each acquired from the same general tissue region, it is desirable to then further register (co-register) each of the images so that changes to tissue features can be tracked over time—and which can be visualized, for example, with a time-lapsed video. It has been found that time-lapsed videos for the assessment of corneal immune cell motility may consisted of four to six time-points separated by four to seven minutes, spanning a total of 25 to 30 minutes—but depending on the application this may change as would be appreciated by those skilled in the art.

Prior to this additional stage of registration, final registered average from each image is reviewed to ensure that a broadly similar area of tissue exists in the field. This was found to be the case for all videos, hence data need note be removed as a consequence of this step. In an embodiment, averaged images are further registered by applying full field translation, applying an affine registration and de-warping.

Full-field translation may be carried out in a similar manner as described with reference to FIG. 90. Here, the threshold for the minimal overlapping fraction is reduced to 25% by area (from 50%), due to the inherently greater variability in alignment when a human participant has moved their head to and from the imaging apparatus between time-lapsed acquisitions. Permitting less image overlap at this stage is possible as the averaged images being registered are much less noisy than the single-frame images that were previously registered, such that spurious peaks in the cross-correlation are less prominent. An increased in a padding parameter to 200 pixels (from 100 pixels as described above) may be appropriate. As described, images are registered in order, based on their shared histogram similarity, to ensure that similar regions of the tissue are registered early in the registration procedure. As also previously described, images are discarded if the required translation exceeds twice the padding parameter (i.e., in this case, 400 pixels), typically indicating a spurious image registration.

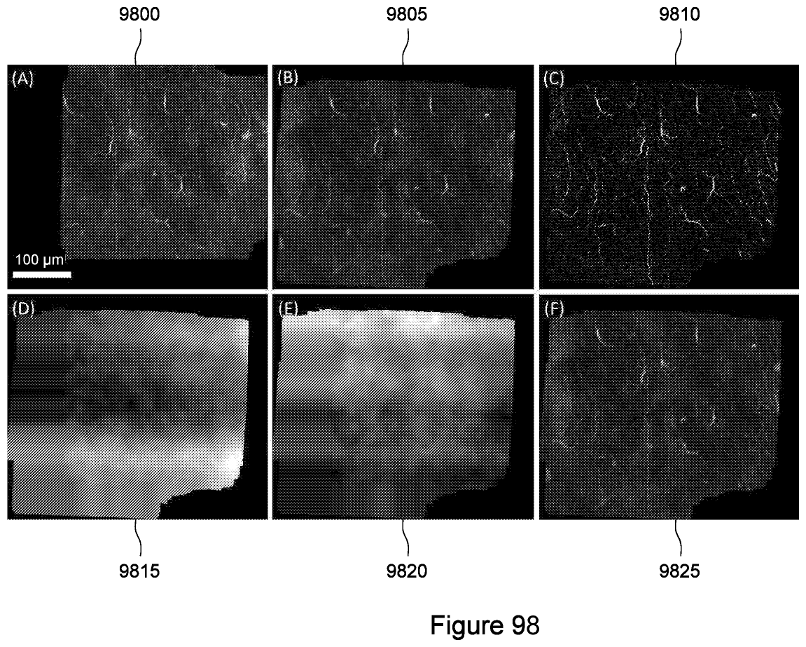

Further registration is performed, which affords correction of rotation due to torsional motion of the head between scans. The registration may include, for example affine registration (or any other suitable registration approach) as would be appreciated by the skilled person. Contrary to torsional motion within a scan, torsional motion between scans was often significant. Other affine transformations, such as scale or shear, are not utilized in this embodiment, although may be utilised in other embodiments, depending on the application. Affine registration may be carried out in any suitable manner (such as the function ‘imregister’ in Matlab or the like). This function uses an optimization-based approach that seeks to maximize the mutual information between the reference image and the registered image. If the proposed registration reduces the correlation between the two images, compared to the unregistered case, the image from that time-point is discarded. FIG. 98A shows an example of the selected reference time point 9800, and FIG. 98B shows an example image from another time point 9805, after appropriate translation and rotation to best match that reference.

The final step of registration involves de-warping the image to be registered, to match the reference image. This stage is preferable to include as the procedure used above to mitigate image distortions, induced by eye movements during raster scanned image acquisition, is imperfect. These residual distortions are preferably corrected to achieve satisfactory alignment of the tissue regions across time points. The appropriate de-warping tool may be selected by utilising a diffeomorphic algorithm (such as the function ‘imregdemons’ in Matlab or the like) such that that it seeks to construct a smoothly varying deformation map—a valuable constraint as it can avoid the introduction of artificial local extrema by using a “smoothing” parameter. In an embodiment, the parameter was set here to 3.0, the maximum value recommended, ensuring that only coarse de-warping of background tissue is applied as opposed to local manipulation that could impart significant changes to cell position. It will be appreciated that, depending on the application, this value may change.

In an embodiment, it may be possible to apply only a diffeomorphic registration (i.e., skipping the full-field translation and rotation steps described above). Skipping each of these steps leads to poorer registration outcomes and distortion artifacts but may be appropriate in some circumstances.

The inventors have found that carrying out diffeomorphic registration provides satisfactory results for registering time-lapsed images in the corneal stroma, which consists of densely-packed cells (approx. 400 cells/mm²) fixed in space, with a small number of motile immune cells (macrophages). However, the stable background tissue seen when imaging the corneal epithelium is much sparser, with bright corneal nerves separated by large amounts of dark tissue. This leads to highly motile immune cells (T cells) dominating the registration procedure in those otherwise dark areas, causing inappropriate local image warp to be inferred by the registration procedure.

Therefore, in an embodiment, a pre-filter may be applied to epithelial images using a “vesselness” filter that enhances tubular structures in images. This filter suppresses the cells that had more of a punctate appearance, leaving the tube-shaped corneal nerves as the major image structures that are input to registration. An example output of this filter is shown in image 9810 of FIG. 98C.

Using the above procedure, deformation maps 9815, 9820 can be produced in the x and y directions, as shown in FIG. 98D and FIG. 98E, respectively. For pixels in the deformation map where there is no overlap with the reference tissue, deformation values are populated with the closest value in the deformation map. This explains the horizontal banded appearance in the maps shown in FIG. 98D and FIG. 98E, due presumably to residual uncorrected distortions in the slow (vertical) scanning direction. The deformation values prescribe a shift of no more than 4 pixels for any piece of tissue, indicating reasonable success of the aforementioned steps to minimize distortion. These deformation maps are used to transform the original image (i.e., without the “vesselness” filter applied), with the output 9825 shown in FIG. 98F.

In an embodiment, the image processing method may be improved by providing a second strip-based image registration and applying it to align the two or more images with the second reference image.

Preferably, at least 100 images are received and processed so as to provide a video. As an example, for a given time-point of a region of biological tissue may include up to 100 images in order to ultimately provide a video sequence as output.

All such averaged images, corresponding to different time points for the same tissue, are then co-registered by a diffeomorphic procedure to produce a time-lapsed video in which cells can be tracked with high fidelity.

### EXAMPLES

Examples illustrating applications of embodiments of the disclosure will now be described. The examples are supplied to provide context and explain features and advantages of the disclosure and are not limiting on the scope of the disclosure as defined in the provisional claims. Examples 1-5 are described with reference to FIGS. 1-8 and 35-78 and the Materials and Methods set forth herein.

### Example 1—Novel IVCM Technique Identifies Immune Cell Subsets and Changes in Inflammatory States

### Materials and Methods

Human participants. Ethical approval was obtained from the University of Melbourne Human Research Ethics Committee (ID #22828). All participants provided written informed consent to participate. Study procedures were performed in the Department of Optometry and Vision Sciences, University of Melbourne, Victoria, Australia.

Participants (n=30) were recruited from advertisements at the University of Melbourne (n=16 healthy controls) and via a notification to register interest about the study in the Melbourne Pollen App, https://www.melbournepollen.com.au/mobile-app/, (n=14 individuals with seasonal allergy). A first, observational cross-sectional analysis compared healthy controls to adults actively experiencing mild to moderate seasonal ocular allergy symptoms without concomitant medical management (n=10, untreated allergy group) to adults with seasonal ocular allergy who had recently had immunomodulatory therapy (n=4, treated allergy group). Participants attended for a study visit in November or December 2021, to align with the peak of the grass pollen season in Melbourne, Victoria, Australia⁸, being the time period expected to provoke a maximal ocular inflammatory response in the seasonal allergy population. Participant testing involved questions to confirm demographic information, and general and ocular health status. Allergy symptoms were quantified using the Eye Allergy Patient Impact Questionnaire⁹.

Eligible participants were aged 18 to 40 years inclusive, in good general health with no self-reported conditions expected to affect eye health (other than seasonal allergy), not pregnant or breastfeeding, had not previously undergone ocular surgery, did not have a history of contact lens wear and had not received a vaccination for COVID-19 or influenza within two weeks of enrolment. Potential participants with a current or prior history of ocular infection or inflammation, or with corneal abnormalities (including a history of ocular injury, keratoconus and corneal dystrophies) were excluded. Individuals with dry eye disease were ineligible to participate; dry eye diagnosis involved the protocol recommended in the 2017 Tear Film and Ocular Surface International Dry Eye Workshop II¹⁰.

Individuals in the untreated ocular allergy group were also required to have not used a topical or systemic antihistamine medication within one week of enrolment, or a topical (including nasal) or systemic corticosteroid or immunomodulatory agent within one month of enrolment. Participants for the contact lens (CL) study comprised a subpopulation (n=4) of the healthy controls (naïve to CL wear, per the study eligibility criteria) who re-attended on a separate day for baseline The inventive IVCM imaging (pre-CL), with repeat imaging after approximately three hours of wear of a soft silicone-hydrogel contact lens (Air Optix Aqua, Alcon, USA).

IVCM imaging. Participants' right corneas were imaged using laser-scanning in vivo confocal microscopy (IVCM), with the Heidelberg Retina Tomograph 3 (HRT3) and Rostock Corneal Module (Heidelberg Engineering GMB, Germany, FIG. 1). Prior to imaging, the eye was anesthetized (Alcaine 0.5% eye drops, Alcon, USA). Volume (z-stack) scans [400(x)×400(y)×100(z) μm], spanning the basal layer of the corneal epithelium to the mid-stroma (FIGS. 2-8), were captured at the infero-nasal paracentral cornea (also known as the corneal whorl). Illumination was set at automatic brightness for all image captures. Capturing the region of interest was achieved by having the contralateral (left) eye focus on a fixation target. Repeated volume scans of this same corneal region were acquired every 4 to 7 minutes for a total of 20 to 40 minutes.

In addition to the volume scans, sequence scans at the level of the basal corneal epithelium (sub-basal nerve plexus) were acquired at the corneal apex to quantify additional cell density parameters. The central corneal sampling area allowed for the analysis of 12 high-quality non-overlapping IVCM images per participants, to provide immune cell density calculations within 30% of the anticipated true mean, 95% of the time¹¹. Per the inventors' laboratory protocols, IVCM images that were blurred, had an inconsistent imaging depth, or showed vignetting effects were removed from all potential analysis sample sets^{12, 13} No post-capture image enhancements were performed.

IVCM video curation and analyses. Building on earlier proof of concept based on single-plane scans at the level of the epithelium¹⁴, time-lapsed IVCM videos were created in ImageJ (version 1.53u, National Institutes of Health, US) by generating stacks that combined five to six time-separated, unique IVCM images of the same corneal region, at a consistent tissue depth (FIG. 8). For analyses at the level of the basal corneal epithelium, nerve axons were used as static features to register the image set, using the TrakEM2 ImageJ plugin. The same procedure was followed to curate time-lapsed videos for the anterior- and mid-stroma, using stationary keratocyte nuclei as landmarks.

Image analyses were performed by the inventors masked to participants' health status, achieved by use of a unique identification code per time-lapsed video. A suite of parameters was used to define the morphology and dynamic behaviours of the corneal immune cell subsets (Table 1). Cell densities were calculated “per participant”, for the central and paracentral corneal regions. All other analyses were performed at the “per cell” level¹⁵.

TABLE 1
Human corneal immune cell parameters quantified from time-
lapsed corneal in vivo confocal microscopy videos

| Cell feature | Category | Specific immune cell feature(s) |
|---|---|---|
| Static cell | Frequency | Density (cells/mm²); |
| features¹⁵ | Morphology | Field area (μm², total area encompassed by the polygon |
| | | defined by the outermost aspects of the dendrites of the |
| | | cell); |
| | | Cell area (μm², total area encompassed by manual |
| | | freehand trace around the cell border) |
| | | Perimeter (μm, distance around the edges of the cell, as |
| | | determined by tracing around the cell border); |
| | | Circularity (4π × [field area/perimeter²]); |
| | | Roundness (4 × field area/[π × major_axis²]); |
| | | Solidity (area divided by convex hull area); |
| | | Dendritic complexity (dendritic tips/cell^{a}). |
| Cell | Motility and | Cell instantaneous speed (μm/min^{b}); |
| dynamics^{17, 19} | dendrite | Displacement speed (net cell displacement divided by |
| | probing | total time^{b}); |
| | | Meandering index (net displacement divided by total |
| | | track length^{b}); |
| | | Outreach ratio (maximum displacement divided by total |
| | | track length^{b}); |
| | | Arrest coefficient (percentage of time cell speed <2 |
| | | um/min^{b}); |
| | | Linearity of forward progression (ratio of the |
| | | displacement speed and mean instantaneous speed^{b}); |
| | | dSEARCH (dendrite surveillance extension and retraction |
| | | cycling habitude^{a})¹⁸; |
| | | Probing speed (total displaced length of each dendrite tip/ |
| | | total time, averaged for all dendrite tips from one cell, |
| | | μm/min^{a}); |
| | | Dancing index (calculated after co-localized macrophage |
| | | cells from two consecutive timepoints (aligning the |
| | | centroids of the two cell shapes), non-overlapped area/ |
| | | total area, normalized to time, and averaged from all time |
| | | intervals for each macrophage^{c}) |

^{a}For DCs only;
^{b}For T cells only;
^{c}For macrophages only;
^{d}For T cells and macrophages only
Legend: min, minute.

Static two-dimensional (2D) immune cell morphological parameters were calculated as described in Table 1^{15, 16, 17}. The analysis involved using the Polygon Selections feature tool in ImageJ, to manually connect points to delineate the outermost aspects of the dendrites of individual immune cells, followed by application of the Analyse—Measure function in ImageJ to quantify cell field area. The borders of the cell were then separately traced using the ‘freehand trace function’ to quantify the following cell shape descriptors: perimeter, circularity, roundness and solidity. For DCs, the number of dendritic tips cell was manually counted as a measure of dendritic complexity.

To analyse immune cell dynamics, two-dimensional cell motility (for T cells) and dendrite probing behaviour (for DCs) data were derived in a stepwise manner using consecutive frames in the time-lapsed corneal in vivo confocal microscopy video. For each parameter, the mean value across the capture period was used as the representative value per cell, normalized per unit of time (minute) where appropriate. Individual cell tracks were manually determined using the ‘Manual Tracking’ function in ImageJ. Cell track parameters (x-y coordinates for the cell centroid, and the inter-frame time interval) were exported as CSV files, to derive cell instantaneous speed, displacement speed, meandering index, outreach ratio, arrest coefficient and the linearity of forward progression (see Table 1 for definitions). All cells that could be reliably followed and had tracks with a minimum of four time points were incorporated in the analyses. To quantify DC dSEARCH (dendrite surveillance extension and retraction cycling habitude)¹⁸, the absolute values of the changes in dendrite length for all dendrites over the capture period was summed to derive the dSEARCH index and normalized per minute (normalized dSEARCH index).

Stromal macrophages are largely sessile, exhibited amoeboid-like cytoplasmic ruffling movements. To identify and quantify macrophage density in the stroma, two-dimensional time-lapsed videos were curated to enable visualisation of dynamic cells compared to stationary keratocytes. For each timepoint, volume scans were used to create z-stacks (typically two to four layers, depending on the consistency of the focal plane) from the anterior-most layers in the stroma. Matched x-y regions were extracted from each time point and then used to create a new stack which was then registered using the Linear Stack Alignment with SIFT-affine function in FIJI, ImageJ (version 1.53u, National Institutes of Health, US). Hyperstacks were created for two-dimensional visualization of the time-dependent behaviours of the stromal cells. Morphometric parameters (i.e., field area, cell area, perimeter, roundness, circularity and solidity, see Table 2) were calculated from the timed lapse videos. To demonstrate the dynamic behaviours of macrophages compared to keratocytes, the co-efficient of variation (based on cell area) was calculated across all time points, and masks were generated to portray the total area occupied by a selection of representative cells over time.

TABLE 2
In vivo morphological characteristics of immune cell subsets in healthy human
corneas quantified from time-lapsed corneal in vivo confocal microscopy images

| | Epithelium | | Stroma | |
|---|---|---|---|---|
| | T cells | DCs | Macrophages | T cells |
| Parameter | (n = 65) | (n = 17) | (n = 26) | (n = 4) |
| Field area (μm²) | 81.6 ± 47.2 | 1171.2 ± 316.7 | 478.3 ± 199.2 | 184.2 ± 84.2 |
| Cell area (μm²) | 55.6 ± 23.6 | 198.4 ± 52.4 | 278.6 ± 60.7 | 111.4 ± 8.4 |
| Perimeter (μm) | 41.4 ± 14.4 | 221.0 ± 88.9 | 100.0 ± 24.1 | 55.76 ± 5.8 |
| Circularity | 0.44 ± 0.13 | 0.04 ± 0.02 | 0.39 ± 0.12 | 0.48 ± 0.11 |
| Roundness | 0.43 ± 0.13 | 0.50 ± 0.23 | 0.45 ± 0.17 | 0.44 ± 0.15 |
| Solidity | 0.73 ± 0.12 | 0.19 ± 0.07 | 0.68 ± 0.10 | 0.76 ± 0.06 |

Data are shown as mean ± SD.

Mice. C57BL/6, gBT-I, B6.PL-Thyla/CyJ (Thy1.1)²⁰, C57BL/6-Tg(UBC-GFP)30Scha/J (uGFP), gBT-I:uGFP and gBT-I:Thy1.1 mice were bred and maintained at the Peter Doherty Institute (Melbourne, Australia). B6.Cg-Tg(Itgax-Venus)1Mnz/J (CD11c-EYFP) and B6.129P2(Cg)-Cx3cr1tm1Litt/J (CX₃CR1^{GFP/+}) mice were bred and maintained at the Florey Institute of Neuroscience and Mental Health (FINMH, Melbourne, Australia). All experimental procedures were approved by the FINMH Animal Ethics committee (18-093-UM) and the University of Melbourne Animal Ethics Committee. For infection of mice to generate corneal memory T cells, Herpes Simplex Virus-1 (HSV-1) KOS strain was used. Mice were adoptively transferred with 5×10⁴ gBT-I CD8⁺ T cells prior to infection. Mice were anaesthetized with a mixture of ketamine/xylazine at 100 mg/kg and 15 mg/kg for infection. One drop of topical anaesthetic (Alcaine 0.5%, Alcon, USA) was placed onto each eye for five minutes and then wicked off using a cotton swab. The corneas were then lightly scratched using a 30 G needle and 10 μL of PBS, containing 10⁶ pfu of HSV, was placed onto the eye.

Mouse corneal imaging. To demonstrate the general dynamic behaviours of distinct cell subsets, time-lapsed movies of corneal epithelial DCs and stromal macrophages were created using explant tissue preparations from CD11c-EYFP and CX3CR1^{GFP/+} mice respectively. Excised corneas were transported to a live-cell imaging chamber open perfusion chamber (Round coverslip chamber; Cat #RC-25F; Harvard Apparatus, MA, USA) with a vacuum grease-sealed coverslip and 500 μL of DMEM F-12. Tissues were imaged in a temperature-controlled chamber (37° C. temperature and 5% C02 air flow) and confocal Z-stacks (×20 objective) were acquired every two minutes over 30 minutes (Leica SP8 Laser Scanning Confocal Microscope). For calculation of static cell morphometry parameters of epithelial CD11c-EYFP+ DCs and stromal CX3CR1^{GFP/+} macrophages (n=50 cells from n=5 mice), intravital microscopy was performed using a laser confocal endoscope (FIVE2 ViewnVivo; Optiscan technologies).

Morphometry of corneal memory gBT-I CD8⁺ T cells (n=30 individual cells from n=9 mice) was analysed using intravital multiphoton microscopy movies. Intravital corneal imaging was performed as previously described¹⁹. Briefly, Mice were anaesthetized with isoflurane (3.0% induction, maintained at 1.5%, vaporised in a mixture of 80:20 oxygen and air). Mice were placed in a head holder (Narashige) and the imaged eye was anaesthetized using one drop of topical anaesthetic (Alcaine 0.5%, Alcon, USA) for five minutes. The eye was gently exposed from the socket and secured using a plastic loop, a cone placed around the eye, sealed with vacuum grease, and filled with PBS. Intravital imaging was performed in a custom-made environmental chamber (Precision Plastics) maintained at 35° C. using heated air. Image acquisition was performed with a Zeiss LSM710 NLO multi-photon microscope (Carl Zeiss Microimaging) using a 20×/1.0 NA water immersion objective. The tissue was excited at 925 nm using a two-photon Ti:sapphire femto-second pulsed laser (Chameleon Vision II, Coherent Scientific). Three dimensional stacks were acquired at 30-40 second intervals for 30-40 minutes. Data and movies were processed using Imaris 8.3.1 (Bitplane).

Mouse IVCM imaging. To capture images of mouse corneas with T cells, eyes from CX3CR1-deficient mice were examined 30 days after HSV infection. Immediately after euthanasia, eyes were fixed in 1% paraformaldehyde for 1 minute and imaged immediately with the Heidelberg Retina Tomograph 3 (HRT-3) with Rostock Corneal Module (Heidelberg Engineering GMB, Germany, FIG. 1). The mouse eye was oriented manually, and volume (Z-stack) scans were taken of the entire corneal thickness at the paracentral cornea. Eyes were lubricated using Genteal Gel (Alcon, USA), and after imaging was completed, eyes were post-fixed and processed for immunofluorescence staining.

Immunostaining. Excised mouse corneas were fixed in 4% paraformaldehyde for up to 1 hour. Following permeabilization with 25 mM ethylenediaminetetraacetic acid for 1 hour at 37° C., the corneas were blocked with a buffer of 3% BSA and 0.3% Triton-X for 1 hour at room temperature and stained with CD45 (1:500; Cat #550539, BD Biosciences, NJ, USA) in PBS overnight at room temperature. Corneas were subsequently washed and stained with CD3-647 (1:200; Cat #317312, Biolegend, CA, USA), Thy1.1-488 (1:500; Cat #202506, Biolegend, CA, USA), goat anti-rat Alexa Fluor 555 (1:500; Cat #A21434, Invitrogen, MA, USA) and Hoechst (1:1000; Cat #14533, Sigma-Aldrich, MO, USA) in PBS for at least 2 hours at room temperature. The stained samples were washed and mounted onto slides with Prolong® Gold. After drying, tile scans of the corneal basal epithelium were acquired using a Zeiss LSM 780 confocal microscope.

Statistical analyses. Statistical analyses were performed in GraphPad Prism (version 9.0, GraphPad Software Inc., California, US). Inter-group comparisons for the study of healthy controls vs untreated allergy vs treated allergy participants were performed using a one-way analysis of variance, following confirmation of normally distributed data using Bartlett's test. Post-hoc tests were performed using Tukey's multiple comparisons test. Data are presented as mean±standard deviation (SD), unless otherwise indicated. Correlations were examined using simple linear regression. For pre-CL vs post-CL comparisons, unpaired t-tests (for unmatched cell data) and paired t-tests (for matched cells) were performed. An alpha of 0.05 was used to define statistical significance.

### Results

### Example 1A—New IVCM Technique Identifies Three Distinct Subsets of Immune Cells in the Healthy Human Cornea

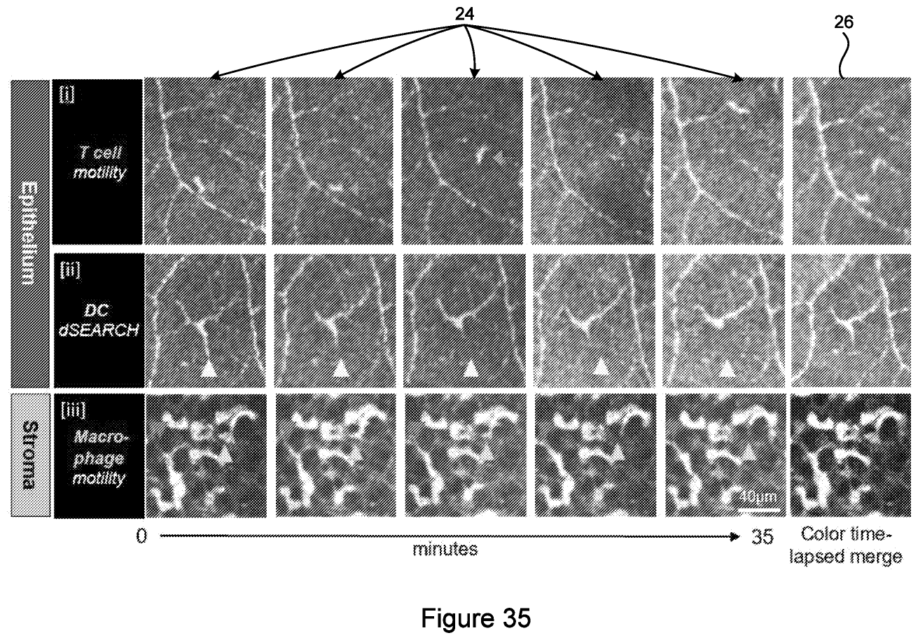

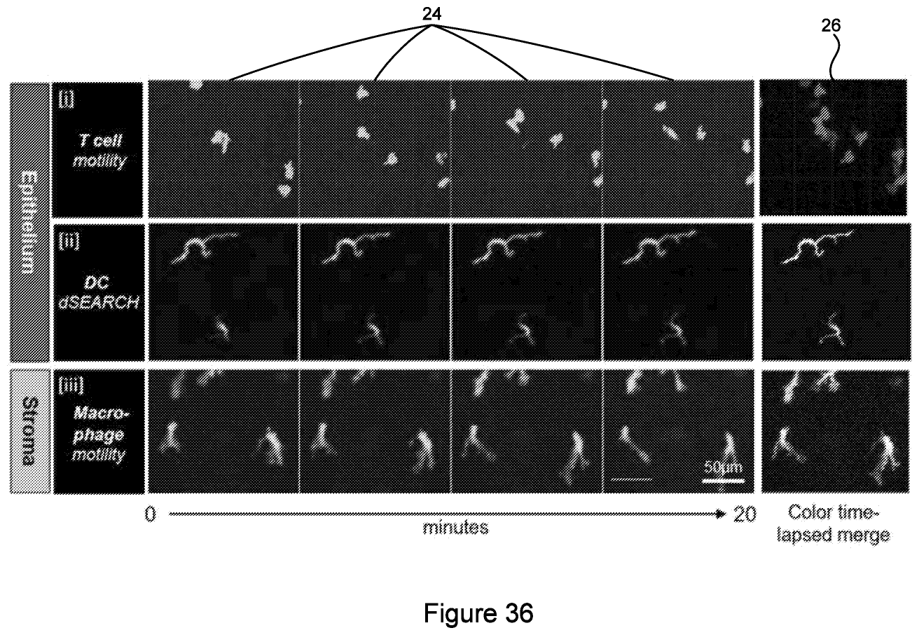

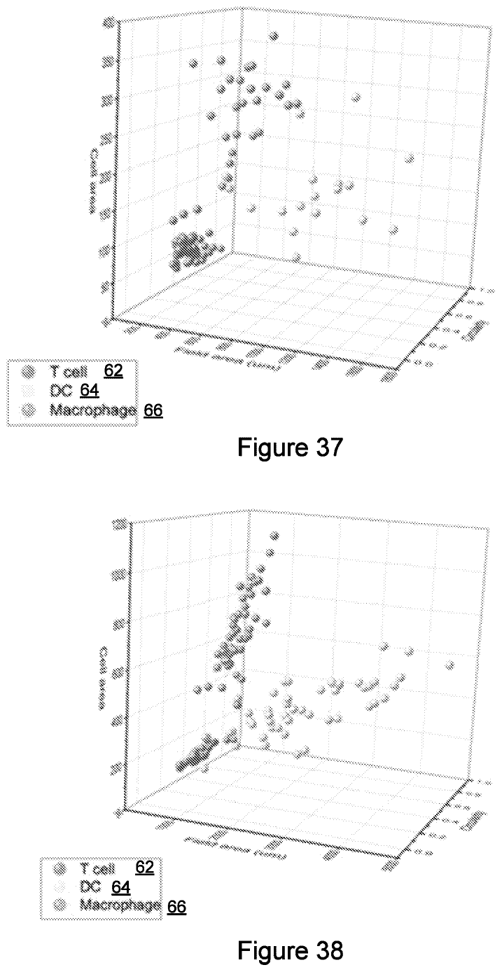

To study the in vivo morphological and dynamic characteristics of human corneal immune cells, the inventors first performed the new IVCM imaging method (FIGS. 35 and 36) in healthy adult participants (n=16; age: mean±SD, 29.7±5.6 years [range: 22 to 39 years]; female/male ratio: 6/10). The new IVCM imaging method involved acquiring corneal IVCM volume scans, spanning the epithelium to mid-stroma (~100 μm depth), at the infero-nasal paracentral cornea (corneal whorl), every four to seven minutes for 20 to 40 minutes. Images at each of the epithelial sub-basal nerve plexus and anterior stroma were registered and reconstructed into time-lapsed videos to track individual immune cells (FIG. 37). The inventors integrated analyses of cell morphology and dynamic behaviour. The inventors linked these in vivo observations in human corneas to the features of corneal immune cells in transgenic fluorescent reporter mice, for comparison of the immune cell classes.

The inventors identified three distinct immune cell subsets in all participants (FIG. 35). Cell morphological features are summarised in Table 2 and FIG. 36. Two immune cell subtypes were identified in the corneal epithelium. The first comprised relatively small, elongated, motile cells (FIG. 35[i]) that appeared to patrol the epithelium. Whilst these cells are typically referred to as “immature DCs” based on human static IVCM imaging⁵, their shape and behaviour mirrored that of GFP+ memory T cells (T_{RM} cells) characterized using intravital 2-photon microscopy in the cornea of live mice after ocular herpes simplex virus (HSV)-1 infection¹⁹ (FIGS. 35, 38). The inventors also confirmed the presence of morphologically comparable immune cells in the basal epithelium of HSV-infected CX3CR1-deficient mouse corneas using static IVCM; CD3 immunostaining of these mouse corneas ex vivo, to confirm their T cell identity, shows morphological homology between the stained T_{RM} cell population, and the immune cells seen using IVCM. These cells are unlikely to be DCs as CX3CR1-deficient mice lack corneal epithelial DCs²⁵. Together, these findings are consistent with the conclusion that the small, motile immune cells that lack visible dendrites in the healthy human corneal epithelium are T cells rather than “immature DCs”.

The second cell subset comprised larger cells with a dendriform shape and obvious dendrites (FIG. 35[ii], 37). The number of dendritic tips ranged from three to nine per cell (mean±SD: 5.0±1.8 tips/cell). Cells with this morphology are routinely termed “mature DCs” in humans based on static IVCM⁵. The inventors have described, for the first time in the living human eye, the dynamics of these cells; they showed minimal lateral migration and repeated, active extension and retraction of their dendrites. Their shape (FIG. 35[ii]) and behaviour were consistent with the probing behaviour of DCs in the corneal epithelium of CD11c-eYFP+ mice (FIG. 37, 38), and DCs in other tissues¹⁸.

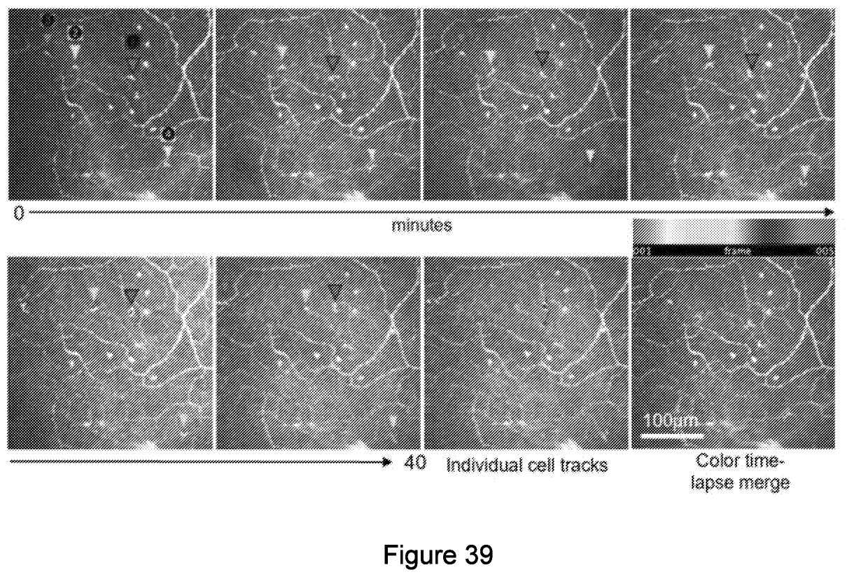

In the human corneal stroma, the inventors observed the dynamics of amoeboid shaped cells with a veiling motion that appear to use membrane extensions to squeeze past presumed keratocyte nuclei (FIG. 35[iii], 37). These cells were only able to be distinguished from stationary stromal landmarks by using the new IVCM imaging technique to reveal their dynamics; static IVCM did not allow for their visualization. The morphology (FIG. 35[iii]) and behaviour of these cells appear analogous to corneal macrophages (FIG. 36[iii]) imaged using in vivo methods in CX3CR1 GFP⁺ mice (FIG. 37, 38). On rare occasions, small, highly motile cells were observed in the stroma that we surmised were T cells, corresponding with fast moving amoeboid cells in the corneal stroma in mice¹⁸. In addition, stationary, hyper-reflective round-oval shaped bodies, termed “globular cells” in the current literature²⁵, were observed in the corneal epithelial whorl region (FIG. 39).

### Example 1B—the Healthy Corneal Epithelium is Patrolled by T Cells, which Show Enhanced Motility and Differential Changes to Cell Density in Chronic Versus Acute Inflammation

The inventors also evaluated whether the new IVCM technique could provide new in vivo insight into the functional effect(s) of chronic immune-mediated disease (ocular allergy, with and without immunomodulatory treatment) and acute ocular surface inflammation (from short-term ocular exposure to a CL) on immune cell subsets. This study involved three age- and sex-matched participant groups (n=30 in total): healthy controls (n=16); individuals with seasonal ocular allergy without concomitant medical management (n=10, untreated allergy); and individuals with seasonal ocular allergy who had recently received immunomodulatory treatments (n=4 treated allergy; comprising n=2 nasal corticosteroids and n=2 allergen immunotherapy).

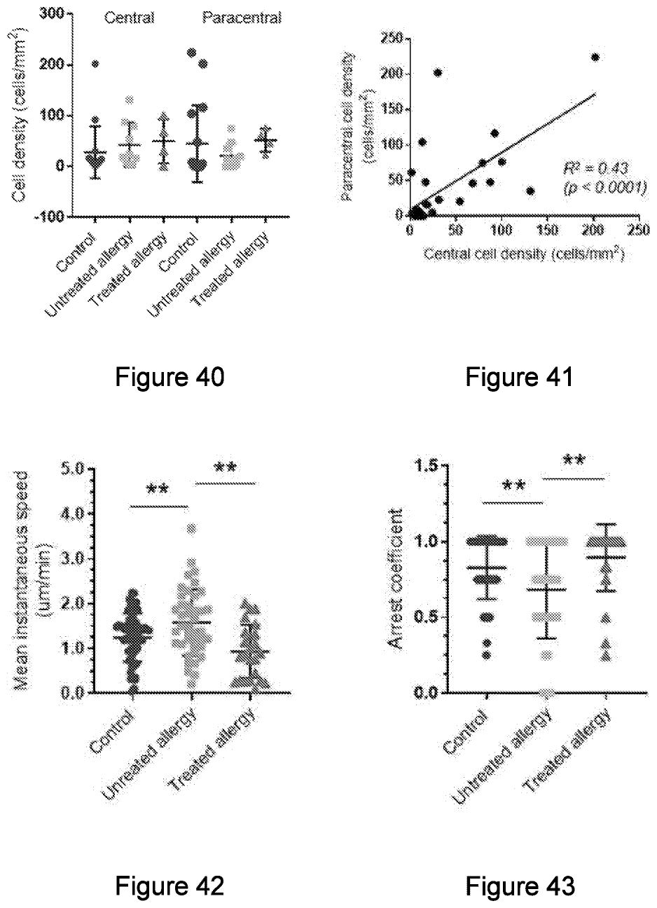

In total, 130 T cells (FIG. 39) were analysed (healthy controls: n=65; untreated allergy: n=41; treated allergy: n=24). These cells were similarly frequent in the central and paracentral cornea in all participant groups (FIG. 40), indicating an inability to differentiate between healthy and disease states based on the traditional static IVCM parameter of cell density. There was a positive correlation between cell density across corneal regions within individuals (FIG. 41, R²=0.43, p<0.0001). Cell motility analyses using the new IVCM imaging technique revealed that in eyes with untreated seasonal allergy, T cells generally had a higher mean instantaneous speed (FIG. 42) and lower arrest coefficient (i.e., proportion of time the cell had a velocity <2 um/min, FIG. 43) than cells in corneas from healthy controls or with ocular allergy treated with immunomodulatory therapy. Other motility parameters, including cell displacement speed, meandering index, displacement ratio, linearity of forward progression and mean directional change were similar between groups (p>0.05 for all comparisons).

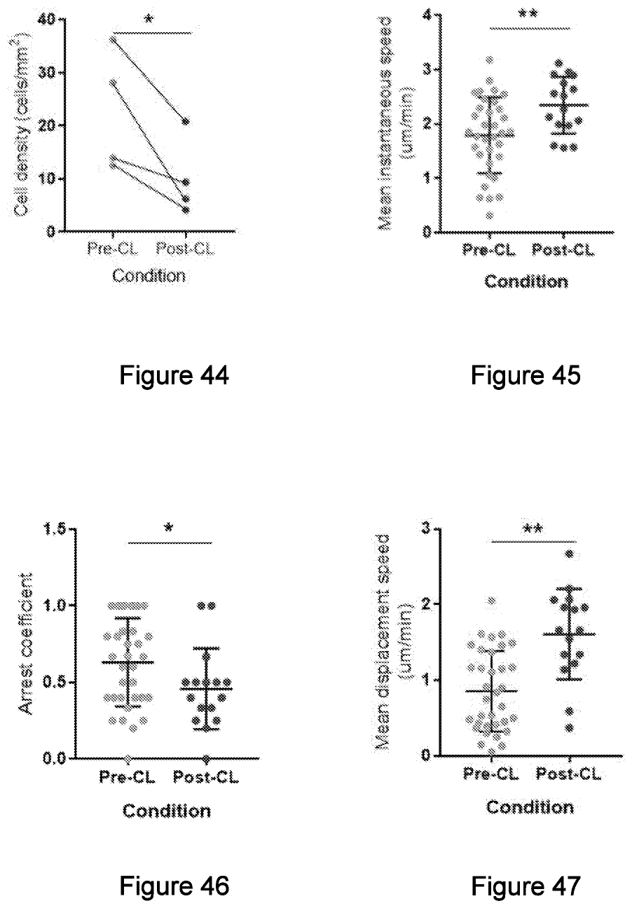

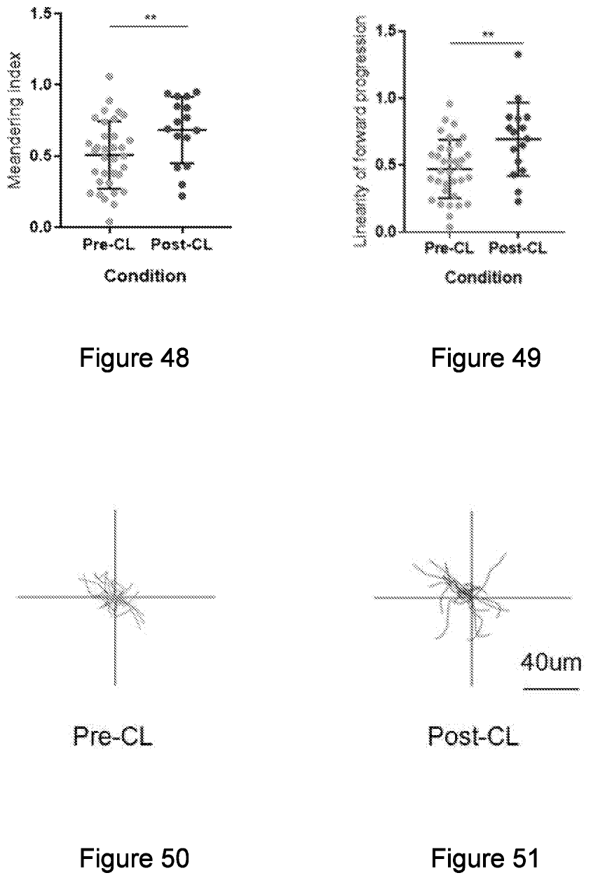

Having identified differences in T cell dynamics between individuals with seasonal allergy and the restorative effect of immunomodulatory therapy on these dynamic parameters, we next examined the effect an acute provocative stimulus in the form of a contact lens (CL), in a subpopulation of the healthy adults. Participants underwent baseline (pre-CL) and repeat new IVCM imaging of the same corneal region after three hours of CL wear (post-CL). After CL wear, the density of epithelial T cells was about half that of pre-CL levels (FIG. 44; mean±SD, pre-CL: 22.7±11.5 versus post-CL: 10.2±7.4 cells/mm², p=0.047). Remaining cells had a higher mean instantaneous speed (FIG. 45), lower arrest coefficient (FIG. 46), higher displacement speed (FIG. 47), higher meandering index (FIG. 48) and higher linearity of forward progression (FIG. 49). Individual cell tracks over a 20-minute period, from the pre-CL (FIG. 50) and post-CL (FIG. 51) time points, provide a visual depiction of the inter-condition differences in the extent of cell motility.

The new IVCM technique identified T cells patrolling the healthy human corneal epithelium. Individuals with untreated ocular allergy had a similar number of T cells, but they had enhanced motility; these altered cell behaviours were normalized in individuals with allergy treated using immunomodulatory agents. An acute inflammatory stimulus induced more pronounced immune cell changes, with reduced T cell density in the paracentral corneal epithelium that suggests active cell migration, and remaining cells showed vastly enhanced tissue surveillance.

### Example 1C—Dynamic Probing Behaviour of Corneal Epithelial DCs Under Homeostatic Conditions is Suppressed by Ocular Surface Inflammation

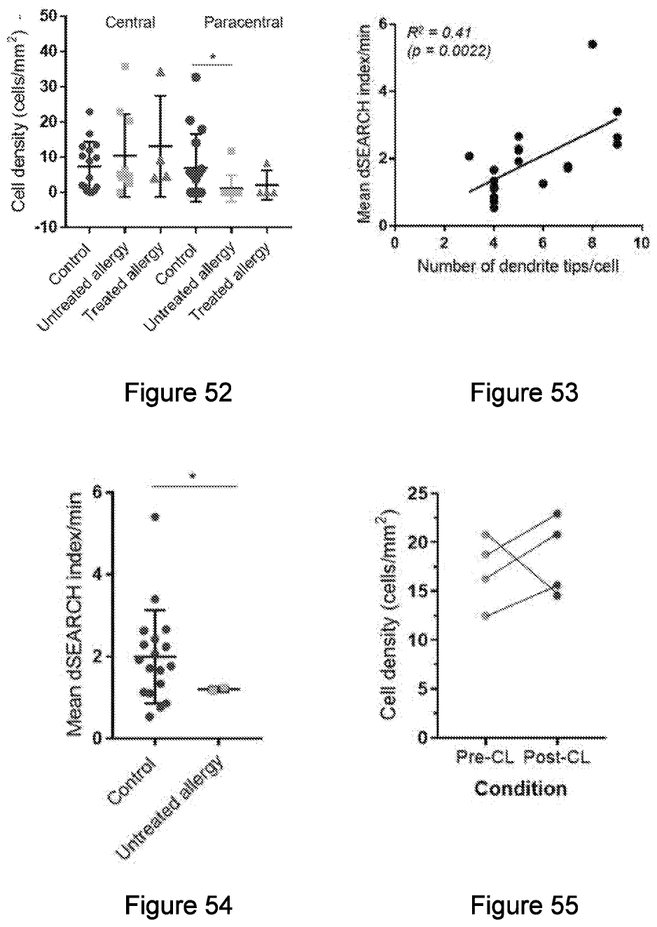

In healthy corneas, putative DCs had similar density in the central and paracentral regions; eyes with seasonal allergy had fewer cells in the paracentral cornea (FIG. 52). Dendrite dynamics were quantified using the dendrite surveillance extension and retraction cycling habitude (dSEARCH)¹⁸. dSEARCH was evident in all 20 imaged DCs, to varying degrees. The inventors quantified the dSEARCH index per minute (normalized dSEARCH index), representing the cumulative distances of dendrite extension and retraction observed for a given DC per unit of time. Cells with more dendritic complexity generally had a higher average dSEARCH index per minute (FIG. 53, R²=0.41, p=0.0022), indicating cells with more dendrites facilitated greater local tissue surveillance. Inter-group comparisons for the normalized dSEARCH index were limited by the presence of very few corneal DCs in eyes with seasonal allergy; the two cells captured showed low dynamic activity (FIG. 54).

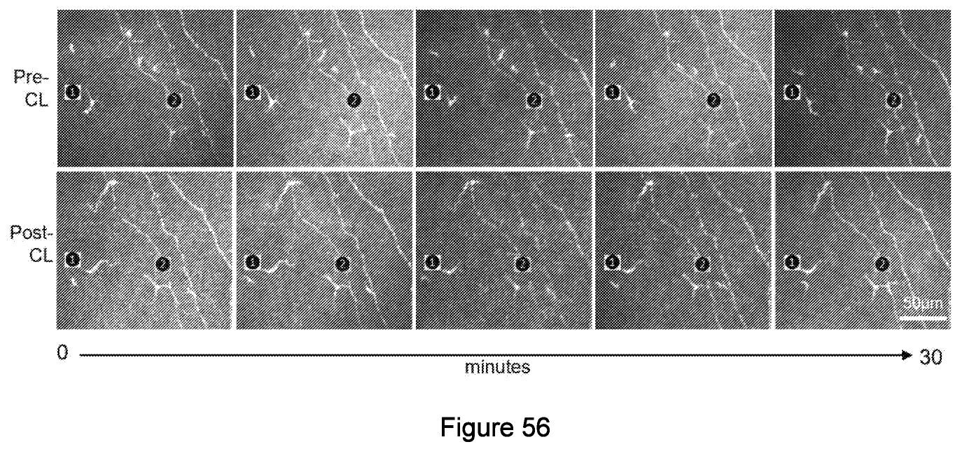

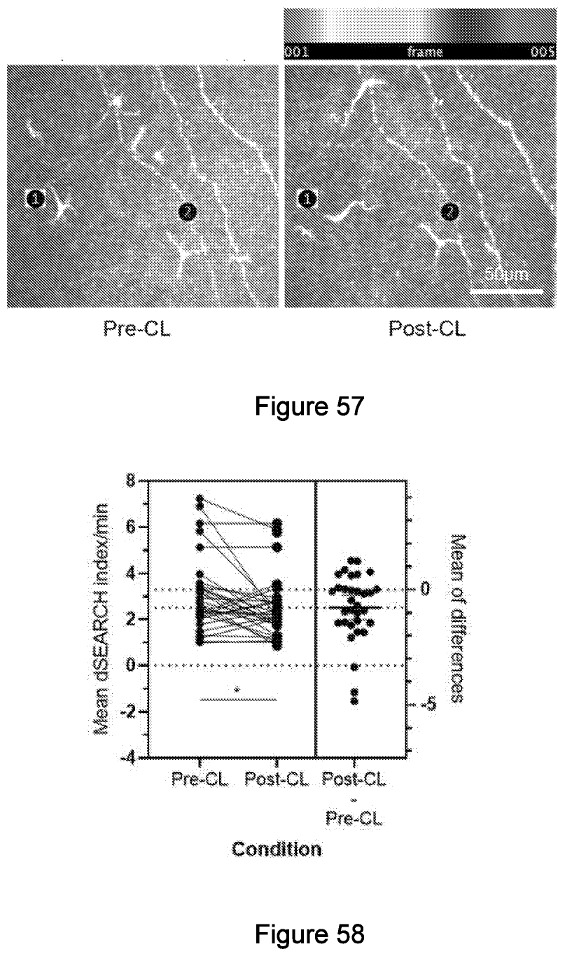

The inventors next sought to assess potential changes to DCs from acute CL-induced inflammation^{21, 27}. At the participant level, DC density did not change between the pre- and post-CL timepoints (FIG. 55). For cell level analyses, the same DCs (n=32) were analysed using repeated measures approaches, by matching cell locations to nerve landmarks (FIG. 56). Post-CL wear, DC dendritic complexity was reduced (mean±SD, pre-CL: 5.0±2.0 versus post-CL: 4.2±1.5 tips/cell, p=0.042). Of the 32 matched DCs analysed, most (75%, n=24) maintained their pre-CL dendritic orientation after CL wear, although some (25%, n=8) showed shifts of at least 90 degrees (FIG. 56-57). Representative colour time-lapsed merges for pre-versus post-CL wear conditions show reduced dSEARCH behaviour after CL wear (FIG. 56-57). The normalized average dSEARCH index was overall lower post-CL wear (FIG. 58, mean±SD, pre-CL: 3.29±2.05 versus post-CL: 2.52±1.41 dSEARCH index/minute, p=0.0052).

In summary, the new IVCM imaging technique enabled quantification of DC dendrite surveillance in healthy human corneas and identified a relative suppression of this constitutive behaviour under non-homeostatic, inflammatory conditions.

### Example 1D—New IVCM Imaging Technique Captures Dynamic Immune Cell-to-Cell and Neuro-Immune Interactions

The inventors also tested using the new IVCM imaging technique to capture in vivo dynamic immune cell-to-cell and neuro-immune interactions in the corneal epithelium. Of 65 T cells tracked in healthy corneas, 22 (34%) appeared to interact with sensory nerves over the capture period. The most frequent type of immune cell-to-nerve interaction was a T cell crossing a nerve (n=17; FIG. 59, green triangle). Less frequently (n=5), T cells appeared to ‘kiss’ nerves, whereby a cell would touch a nerve fibre and then rebound towards the area it approached from (FIG. 59, blue triangle). The average speed of the cell on its initial approach was less than its speed after the nerve interaction (FIG. 60; mean±SD, before: 1.25±0.83 versus after: 1.92±0.61 μm/min, p=0.043), suggesting potential modification to T cell behaviour from the nerve interaction. T cell-to-T cell interactions were visualized infrequently; a representative example of this form of contact is provided in FIG. 61.

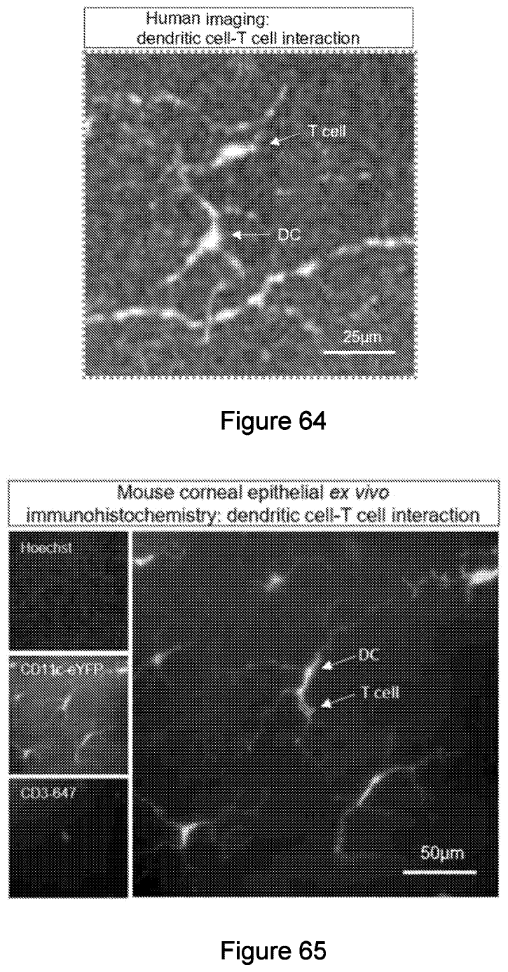

The inventors also captured T cells transiently contacting DC dendrites (FIG. 62-65). A similar interaction, with a CD11c⁺ DC contacting a CD3⁺ T cell, is shown in the mouse corneal epithelium using wholemount ex vivo immunohistochemistry (FIG. 65). Of the 18 DCs quantified from control eyes, six cells (33%) dynamically extended their dendrites to interact with sensory nerves (FIGS. 64-65).

### Example 1E—the New IVCM Imaging Technique Reveals Dynamic Macrophage-Like Cells in the Stroma that are Morphologically Different in Individuals with Allergy, and are Altered by Contact Lens Wear

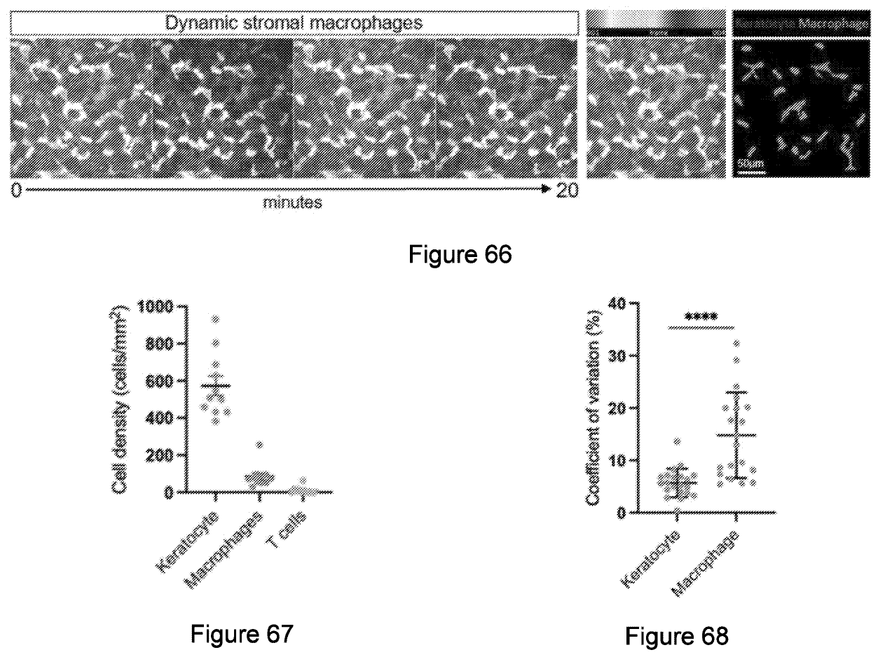

Time lapsed imaging of the anterior stroma revealed the presence of motile amoeboid-shaped cells, that closely resembled the dynamic crawling and lamellipodial features of macrophages observed in the corneal stroma of CX3CR1-gfp mice (FIGS. 35-38). These large, hyper-reflective cells (FIG. 66, orange arrowheads) were in the anterior-most layers of the stroma, adjacent to stationary keratocytes, which are resident fibroblast-type cells of the cornea (pink arrows). Compilation of cell area masks over the imaging period (FIG. 66) showed overt differences in the morphology of the motile cell population (cyan), compared to the stationary keratocytes (magenta). Quantitative analysis of the fluctuations in the area of individual cells over time (as measured by the coefficient of variation, FIG. 68) demonstrated the greater extent of variability in cell morphology in the macrophage-like cells (14.8%, n=20 cells) compared to the relatively homogenous keratocytes (5.6%, n=21 cells, p<0.0001).

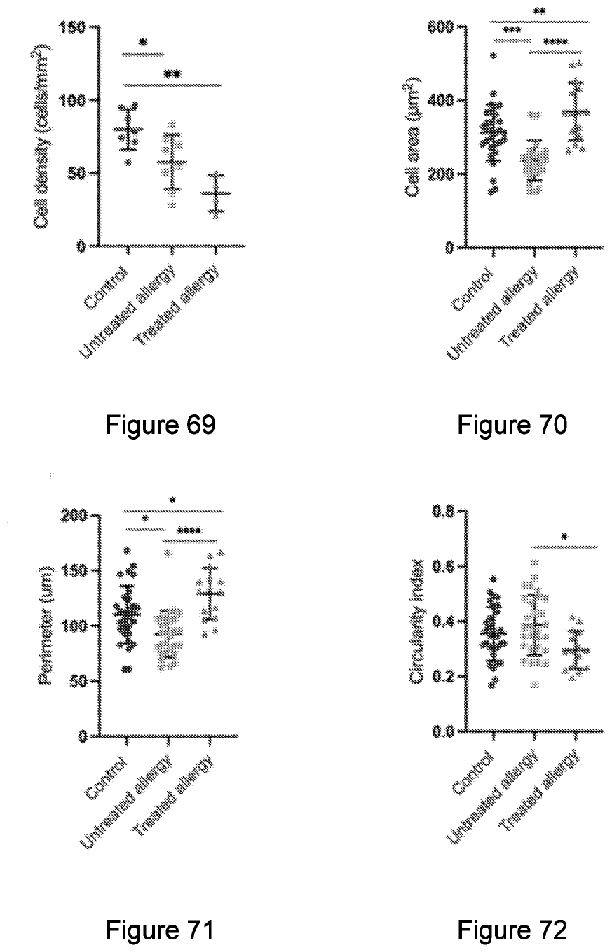

In the healthy human corneal stroma (FIG. 67), stationary keratocytes were the most abundant cell type (573.5±51.9 cells/mm²), followed by putative macrophages (85.9±18 cells/mm²). In less than half of participants, low numbers of highly motile T cells (classified based on their migratory speeds and morphological features that closely resembled epithelial T cells) were observed (10.0±5.6 cells/mm²; FIG. 68). In individuals with treated and untreated allergy, the density of stromal macrophages was lower compared to healthy controls (control vs untreated allergy, p=0.043; control vs treated allergy, p=0.004; FIG. 69). In addition to differences in cell density, macrophages in the allergy group had a smaller cell area (FIG. 70; mean±SD, 237±54 μm²) and perimeter (FIG. 71, 92±21 μm) compared to healthy controls (cell area 312.1±76 μm²; p<0.0002, perimeter 10.2±26 μm, p<0.0001). Cells in the treated allergy group were larger than in both the healthy and allergy groups (cell area 369.8±78 μm²; perimeter 129.2±23.2 μm; FIG. 71), and also had a lower circularity index (0.29±0.07 [allergy] vs 0.35±0.1 [control] vs 0.38±0.1 [allergy untreated] as shown in FIG. 72).

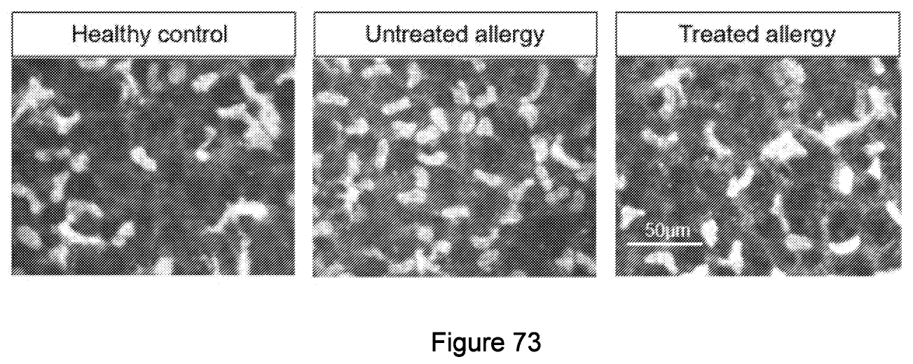

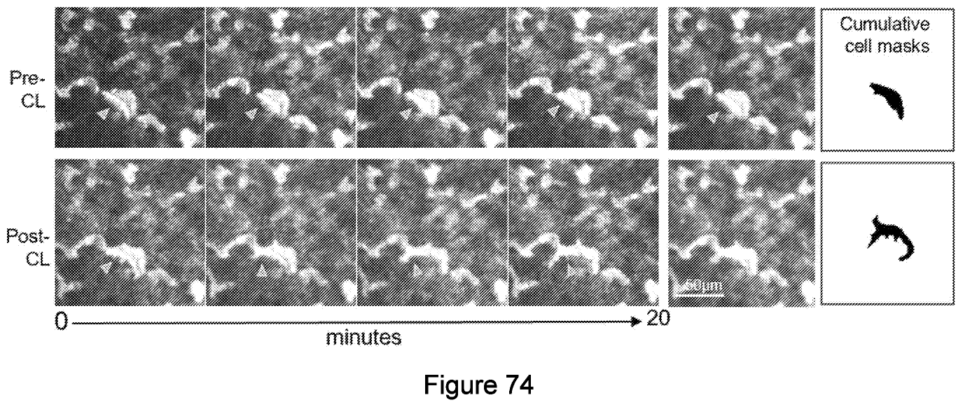

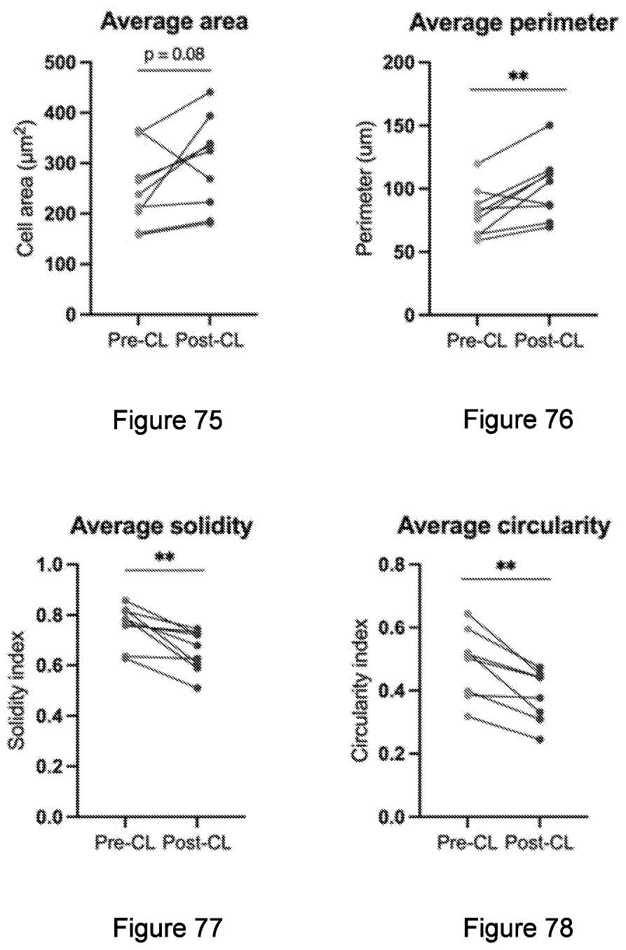

To examine whether an acute inflammatory stimulus altered stromal macrophage features, matched cells from the same corneal location were measured before (pre-CL) and three hours after a CL was applied (post-CL) to the eye (FIG. 73). Colour-coded depth projections and cumulative cell masks depict the total area covered by a representative cell after 20 minutes of imaging and show a higher number of cellular protrusions at the post-CL time-point (FIG. 74). Quantitative repeated measures analyses were performed on a total of nine matched cells from four individuals (FIG. 75-78). Compared to baseline (pre-CL), stromal macrophages after CL wear were larger (pre- vs post-CL wear: cell area, p=0.085; cell perimeter, p=0.009), had lower solidity (p=0.003) and a reduced circularity index (p=0.001).

### Example 2—Evidence for the Ability to Use the Imaging Method to Track the Effect of Environmental Seasonal-Driven Factors on the Density, Morphology and Dynamic Behaviour of Human Corneal Immune Cell Subsets

### Materials and Methods

Human participants. Ethical approval was obtained from the University of Melbourne Human Research Ethics Committee (HREC ID #22828). Written informed consent to participate was obtained from all participants.

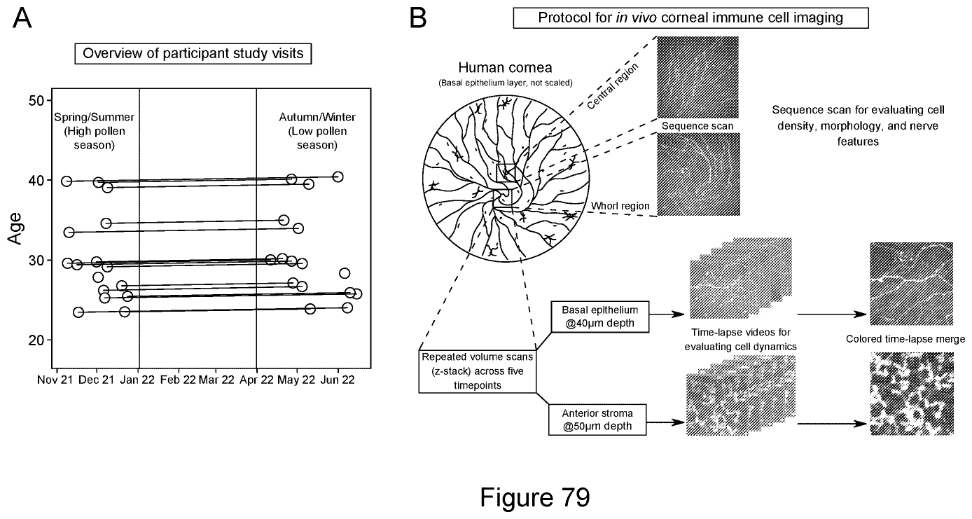

16 healthy participants (10 females, 6 males) in good general health, aged 18 to 40 years inclusive were recruited. All participants attended two visits that occurred about six months apart, over different seasons. Visit 1 (Spring/Summer) was in November or December 2021, aligning with the peak grass-pollen season in Melbourne, Victoria, Australia. Visit 2 (Autumn/Winter) was conducted in April to June 2022, coinciding with the lowest grass-pollen levels (FIG. 79A). All visits occurred between 09.30 AM and 02.30 PM.

Participant inclusion criteria included an absence of current or recent ocular infection or injury, no history of eye surgery, no previous contact lens wear, an absence of any eye or systemic condition(s) that may affect ocular surface health, and not having had a COVID-19 or influenza vaccine within one week of both study visits. In addition, participants were required to have had no history of asthma, sinusitis, dust mite allergy, autoimmune or atopic disease.

Environmental data collection. Daily environmental data were collected over each study period for Visits 1 and 2. These data included: (i) hourly data for temperature (° C.), relative humidity (%) and daily rainfall (mm) from the Australian Bureau of Meteorology Melbourne (Olympic Park) weather observation station; and (ii) PM2.5 and PM10 (μg/m³) air pollutant concentrations from the Alphington (Victoria, Australia) air monitoring site, obtained from the Environment Protection Authority Victoria. Daily grass concentrations were measured using a Burkard volumetric trap (Burkard Manufacturing Co. Ltd., Rickmansworth, Hertfordshire, UK) as previously described.⁵³ The Melbourne sampling site was located at the University of Melbourne, 1.7 km north of central Melbourne at coordinates −37.7971 and 144.9648. Samples were collected at 09:00 (local time) with the measured concentration representing the 24-hour average concentration from 09:00 the previous day in pollen grains/m3 of air. The measured concentration is associated with the previous day the measurement was made, as it covers 62.5% of the measurement period. Daily grass pollen concentrations were categorized into the following levels: ‘low’ for an average concentration between 0 and 19 grains/m3, ‘moderate’ for 20 to 49 grains/m3, ‘high’ for 50 to 99 grains/m3, and ‘extreme’ for concentrations of 100 grains/m3 or more.⁵⁴

Ocular surface examination and time-lapsed corneal in vivo confocal microscopy imaging. All participants completed the Ocular Surface Disease Index (OSDI) questionnaire to evaluate for dry eye symptoms (score range, 0 to 100; score <13 no symptoms, 13-22 mild, 23-32 moderate, >33 severe). To evaluate the frequency of eye rubbing, participants were asked to report how frequently they recalled rubbing their eyes over the past week (score range: never=0, rarely=1, sometimes=2, often=3, always=4). Participants underwent bilateral ocular surface clinical examinations including tear osmolarity, non-invasive tear break-up time (NITBUT), corneal fluorescein staining and conjunctival lissamine green staining (Oxford scale⁵⁵). Conjunctival staining score was calculated as the average from the temporal and nasal conjunctival areas. Ocular bulbar redness (averaged from the temporal and nasal regions), limbal redness (averaged from the temporal and nasal regions), eyelid redness (upper eyelid) and eyelid roughness (upper eyelid) were evaluated using the Brien Holden Vision Institute grading scale. Lid wiper epitheliopathy was graded using the Korb scale⁵⁶. Data from right eyes were used for all analyses, to match the eye that underwent corneal imaging.

Participants' right corneas were imaged using the Heidelberg Retina Tomograph-3 with Rostock Corneal Module, Heidelberg Engineering, Germany, per our protocol.⁵⁷ In brief, the eye to be imaged received one drop of topical anaesthetic prior to imaging. Repeated volume (z-stack) scans [400(x)×400(y)×80(z) μm] spanning the basal epithelium to mid-stroma were captured over a time interval up to 35 mins, at the corneal whorl (inferonasal paracentral cornea) for the latter half of enrolled participants (n=8). In addition, sequence scans, comprising 100 consecutive images acquired at the same corneal depth, were captured at the level of basal epithelium for both the corneal central and whorl regions, to quantify immune cell density and sensory nerve parameters (FIG. 79B).

Time-lapsed video curation and analyses. Time-lapsed, immune cell videos were created using ImageJ software by generating stacks that combined four to five time-separate, unique images of the same corneal region, at a consistent tissue depth. For the epithelium, nerve axons were used as static landmarks to register the image set using the TrakEM2 plugin. For the stroma, time-lapsed videos were created just below the anterior limiting lamina (corneal depth of 50 μm) (FIG. 79B) using the Linear Stack Alignment with SIFT plugin.

Researchers masked to the study visit performed the immune cell analyses, using a suite of parameters to define the morphology and dynamic behaviours of each cell subset. Immune cell subsets were identified according to their distinct morphology and dynamic behaviours, as described previously.⁵⁷ Intraepithelial immune cells with multiple long dendrites were considered dendritic cells (DCs); smaller, motile cells without dendrites were classified as T cells. Macrophages in the stroma were identified in the time-lapsed videos by their crawling behaviour, distinct from static keratocytes, which showed minimal displacement of the hyper-reflective nucleus. For intraepithelial immune cells, eight to 12 non-overlapping static IVCM images were randomly selected from the acquired sequence scan sets to quantify cell density and morphology.⁵⁸ The evaluated cell morphological parameters included field area, cell area, perimeter, circularity, solidity and dendritic complexity (Table 1). Stromal macrophage density and morphology were evaluated from the time-lapsed videos.

Dynamic cell behaviours were evaluated for parameters relevant to the immune cell subtype (Table 1). For DCs, dendrite dynamics were quantified using the dendrite tip probing speed and dendrite surveillance extension and retraction cycling habitude (dSEARCH).¹⁸ The dSEARCH index is the cumulative distance of dendrite extension and retraction observed for a given DC, per unit of time. Dendrite tip probing speed was defined as the averaged distances of dendrite extension and retraction from all dendrite tips for a given DC, per unit of time. For macrophages, the ‘dancing index’ was used to evaluate the extent to which a cell's shape changed between two timepoints.⁵⁹ After aligning the centroids of the macrophages at each timepoint, the ‘dancing index’ was measured by their non-overlapped area divided by the total area, normalized to time. Data for immune cell densities, T cell motility parameters, DC dSEARCH index, and macrophage morphology at Visit 1 only have been reported previously.⁵⁷

Corneal nerve parameters in the central and whorl regions of the cornea were quantified using the automated ACCMetrics software, using the same IVCM image sets as for the intraepithelial immune cell density quantifications. The quantified nerve parameters were: corneal nerve fiber density (CNFD, density of total nerve fibers, fibers/mm²), corneal nerve branch density (CNBD, density of the main nerve fiber branches, branches/mm²), corneal nerve fiber length (CNFL, length of the nerve fibers, mm/mm²), and corneal nerve total branch density (CTBD, density of total nerve fiber branches, total branches/mm²).

Statistical analysis. Statistical analyses were performed using R software (Version 4.3.0, R Development Core Team, https://www.r-project.org/). Immune cell morphological and dynamic parameters were analyzed at the “per cell” level. A linear mixed-effects model was fitted to test the effect of season, using a restricted maximum likelihood methodology. A random effect was included to account for the non-independence between observations from the same participant. For non-parametric data, Wilcoxon signed-rank test was performed. Data are shown as mean±standard deviation (SD), unless otherwise indicated. A p-value less than 0.05 defined statistical significance.

### Results

The inventors evaluated meteorological data in Melbourne, Victoria, Australia across each of the study visits are summarized in Table 3. Visit 1 (Spring/Summer) had higher temperatures (p<0.001 for both maximum and minimum temperatures), lower humidity (p<0.001), higher 10 μm particulate matter concentrations (PM10; p=0.012) and higher concentrations of grass pollen (p<0.001) relative to Visit 2 (Autumn/Winter). Notably, 2.5 μm particulate matter concentrations were not statistically significantly different during the two visits. The average atmospheric grass pollen concentration was categorized as ‘high’ for Visit 1 and ‘low’ for Visit 2.

TABLE 3
Daily meteorological and air quality parameters in Melbourne,
Victoria, Australia during the two study visit periods (Visit
1: November to December 2021; Visit 2: April to June 2022).

| | Visit 1 | Visit 2 | |
|---|---|---|---|
| | (Spring/ | (Autumn/ | |
| | Summer) | Winter) | p-value |
| Maximum temperature (° C.) | 21.9 ± 5.8 | 16.8 ± 3.9 | p < 0.001 |
| Minimum temperature (° C.) | 12.8 ± 2.5 | 10.2 ± 3.2 | p < 0.001 |
| Relative humidity (%) | 65.5 ± 9.2 | 75.4 ± 10.3 | p < 0.001 |
| Rainfall (mm) | 2.1 ± 6.6 | 1.6 ± 4.2 | p = 0.127 |
| PM_{2.5} (μg/m³) | 6.1 ± 3.0 | 7.4 ± 4.5 | p = 0.209 |
| PM₁₀ (μg/m³) | 16.1 ± 6.3 | 14.0 ± 6.4 | p = 0.012 |
| Grass pollen concentration | 64.8 ± 73.8 | 6.5 ± 8.1 | p < 0.001 |
| (grains/m³ of air) | | | |

PM_{2.5}, particulate matter with aerodynamic diameter ≤2.5 μm; PM₁₀, particulate matter with aerodynamic diameter ≤10 μm. Categories of grass pollen concentrations: low (0-19 grains/m³), moderate (20-49 grains/m³), high (50-99 grains/m³), extreme (≥100 grains/m³).

All participants attended both study visits. Demographic and ocular characteristics are summarized in Table 4. Similar findings were observed for clinical ocular surface signs across the visits. Corneal nerve characteristics are summarized in Table 4. The inventors found that there were no changes to corneal nerve parameters between study visits.

TABLE 4
Demographic and ocular surface characteristics
of the study participants at two visits.

| | Visit 1 | Visit 2 |
|---|---|---|
| | (Spring/ | (Autumn/ |
| | Summer) | Winter) |
| Age (years) | 29.7 ± 5.5 | per Visit 1 |
| Sex (male/female) | 6/10 | per Visit 1 |

Clinical characteristics

| | | |
|---|---|---|
| Dry eye symptoms (OSDI score, /100) | 3.3 ± 2.8 | 3.0 ± 2.4 |
| Frequency of eye rubbing (score 0-4)\hat{ } | 1.1 ± 0.8 | 0.9 ± 0.7 |
| Tear osmolarity (mOsmol/L) | 306.6 ± 14.8 | 299.1 ± 10.6 |
| NITBUT (secs) | 10.6 ± 3.4 | 9.6 ± 2.2 |
| Corneal NaFl staining (score 0-5) | 0.6 ± 0.6 | 0.8 ± 0.9 |
| Conjunctival staining (score 0-5) | 0.3 ± 0.6 | 0.2 ± 0.3 |
| Bulbar redness (score 0-4) | 1.0 ± 0.6 | 1.1 ± 0.4 |
| Limbal redness (score 0-4) | 0.5 ± 0.5 | 0.5 ± 0.4 |
| Eyelid redness (score 0-4) | 1.2 ± 0.5 | 0.9 ± 0.3 |
| Eyelid roughness (score 0-4) | 0.1 ± 0.2 | 0.3 ± 0.4 |
| Lid wiper staining score (score 0-3) | 0.2 ± 0.2 | 0.3 ± 0.3 |

Corneal nerve parameters in central region

| | | |
|---|---|---|
| CNFD (fibers/mm²) | 21.5 ± 7.0 | 21.4 ± 7.0 |
| CNBD (branches/mm²) | 28.5 ± 18.0 | 25.6 ± 14.7 |
| CNFL (mm/mm²) | 12.7 ± 3.8 | 13.1 ± 3.6 |
| CTBD (total branches/mm²) | 41.4 ± 24.9 | 38.0 ± 21.9 |

Corneal nerve parameters in whorl region

| | | |
|---|---|---|
| CNFD (fibers/mm²) | 17.5 ± 8.9 | 18.0 ± 8.2 |
| CNBD (branches/mm²) | 58.6 ± 40.6 | 54.7 ± 38.4 |
| CNFL (mm/mm²) | 15.6 ± 5.2 | 15.1 ± 4.0 |
| CTBD (total branches/mm²) | 102.9 ± 65.9 | 94.1 ± 60.7 |

OSDI, ocular surface disease index; NITBUT, non-invasive tear break-up time; NaFl, sodium fluorescein; CNFD, corneal nerve fiber density; CNBD, corneal nerve branch density; CNFL, corneal nerve fiber length; CTBD, corneal nerve total branch density.
\hat{ }Scored on a scale where 0 = Never, 1 = Rarely, 2 = Sometimes, 3 = Often and 4 = Always.

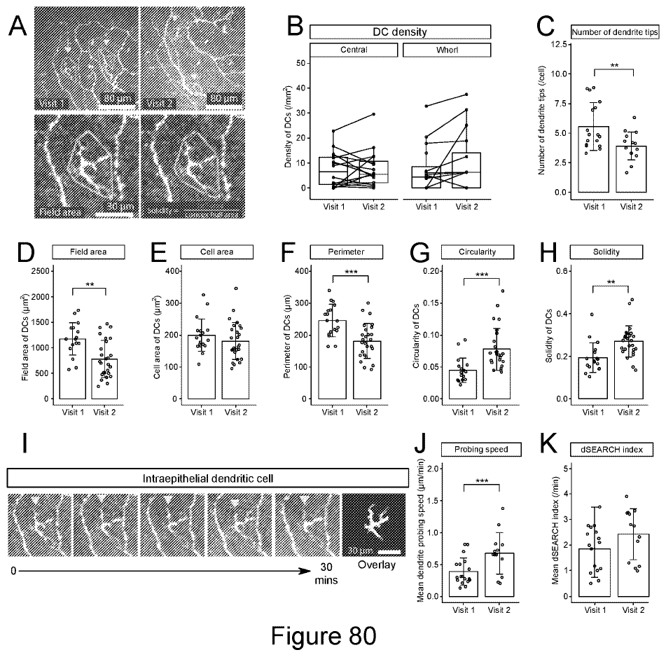

The inventors sought to evaluate corneal epithelial DCs morphodynamics. For corneal epithelial DCs (FIG. 80A), cell density was similar across study visits in both the central and whorl regions (FIG. 80B). The morphology of DCs was affected by season, with DCs generally having a larger, more elongated and more concave shape, with greater dendritic complexity, during Visit 1 (Spring/Summer) relative to Visit 2 (Autumn/Winter) (FIG. 80C-H).

DCs showed a lower dendrite tip probing speed at Visit 1 (p<0.001) relative to Visit 2, but similar normalized dSEARCH index (FIG. 801-K). The larger DC field area and higher dendritic complexity at Visit 1 may compensate for the lower dendrite tip probing speed to achieve similar local tissue surveillance.

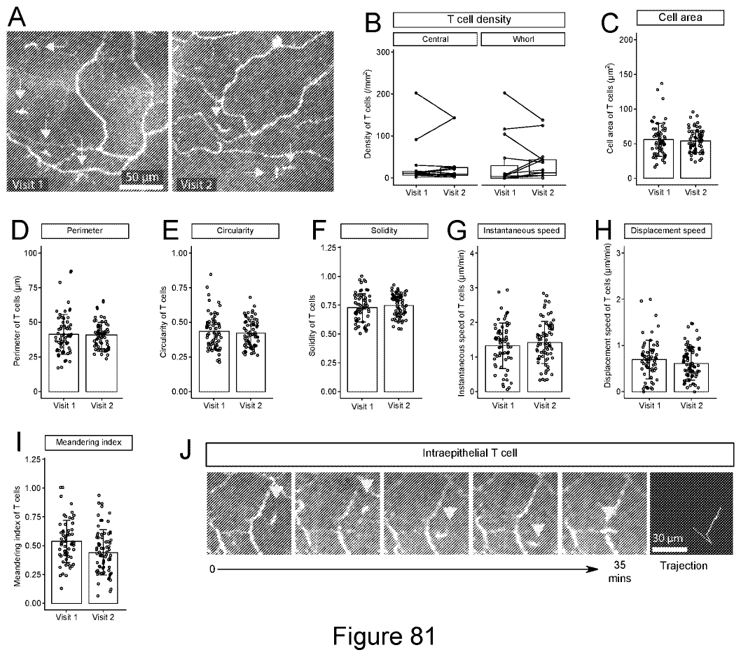

The inventors saw no difference in corneal intraepithelial T cell density across study visits (FIG. 81A-B). Overall, season had no effects on T cell morphology (FIG. 81C-F) or motility (FIG. 81G-J).

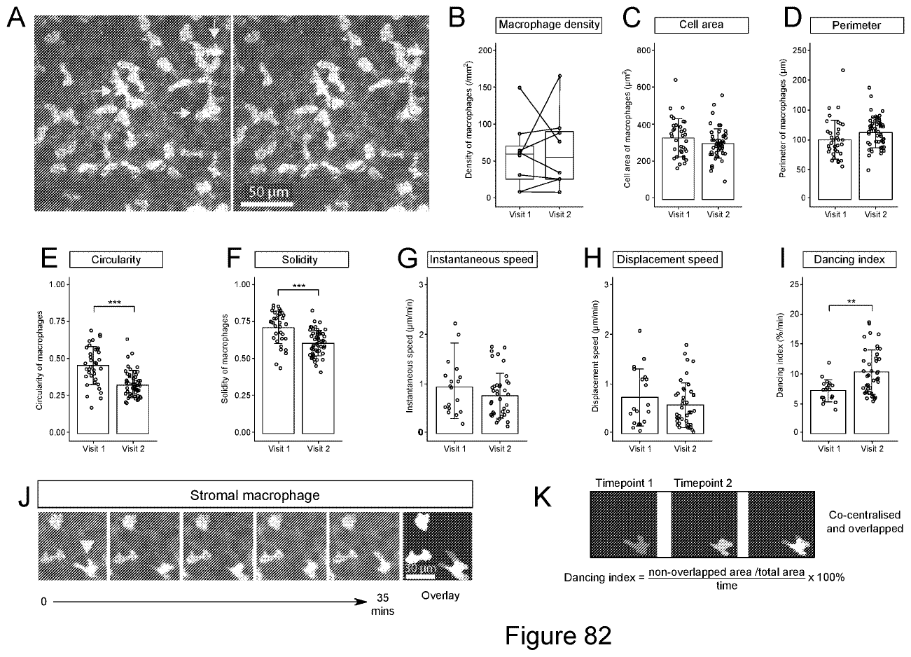

The inventors also found that season had no effect on corneal stromal macrophage density (FIG. 82A-B). At Visit 1, macrophages were more circular and convex in shape (FIG. 82C-F), and had lower dynamic activity (quantified using the ‘dancing index’) relative to Visit 2 (FIG. 82G-K).

While the external environment has been shown to shape the systemic human immune landscape, defining the in vivo immune status of peripheral tissues has remained a technical challenge. Utilising the new IVCM technique, the inventors uniquely described the influence of season on immune cell behaviours in situ in a living human cohort. Season-dependent differences in cell morphodynamics were most apparent in innate immune cells (intraepithelial DCs and stromal macrophages). These findings identify using time-lapsed corneal imaging as a valuable, intact tissue for investigating the effect of external environmental factors on the human immune system, enabling longitudinal quantification of the in vivo morphology and dynamic behaviour of distinct immune cell subsets.

### Example 3—Evidence for the Ability to Use the Imaging Method to Capture Temporal (Diurnal) Changes in Immune Cell Behaviours

### Materials and Methods

Human participants. Ethics approval was obtained from the University of Melbourne Human Research Ethics Committee (HREC ID #23535). Written informed consent to participate was obtained from all participants. Healthy adult participants in good general health, aged 18 to 45 years were recruited. All participants attended two visits that occurred on the same day and separated by approximately eight hours (Visit 1—morning [AM] between 7-9 μm, and Visit 2—afternoon/evening [PM] between 5-7 μm).

Participant inclusion criteria included an absence of current or recent ocular infection or injury, no history of laser refractive eye surgery, have worn contact lenses for more than one month in the past and not within six months of the study, an absence of any eye or systemic condition(s) that may affect ocular surface health (including dry eye disease), not have persistent COVID-19 symptoms (i.e., for >3 months after contracting the virus), and not having had a COVID-19 or influenza vaccine within one week of the study visit.

Ocular surface examination and time-lapsed corneal in vivo confocal microscopy imaging. At Visit 1, participants underwent comprehensive ocular surface examinations, involving collection of information relating to dry eye symptoms (Ocular Surface Disease Index (OSDI) questionnaire), vision level (best-corrected visual acuity), tear film health and corneal health. Basal tear samples were collected for compositional analyses.

Participants' right corneas were imaged using the Heidelberg Retina Tomograph-3 with Rostock Corneal Module, Heidelberg Engineering, Germany, per our protocol.9 The eye to be imaged received one drop of topical anaesthetic prior to imaging. IVCM sequence scans were repeatedly performed at the same corneal region, five-minutes apart, at up to six timepoints. Time-lapsed videos (25 minutes) were created using a customised software package in MATLAB. Cell analyses were performed in Image J. A suite of parameters was used to define the morphology and dynamic behaviours of the immune cell subsets.

Tear cytokine analysis. Cytokine levels in basal tears were analyzed using bead-based multiplex immunoassay (Immune Monitoring 65-Plex Human ProcartaPlex Panel, ThermoFisher, Waltham, MA, USA) and performed according to the manufacturer's instructions using 3 μl of tear sample diluted in 22 μl of assay buffer. Samples were processed and acquired in the same analytical run on the FlexMAP 3D platform (Luminex). Concentration data for each cytokine were interpolated using ThermoFisher ProcartaPlex software.

Statistical analysis. Statistical analyses were performed using R software (Version 4.3.0, R Development Core Team, https://www.r-project.org/). Immune cell morphological and dynamic parameters were analyzed at the “per cell” level. A linear mixed-effects model was fitted to test the effect of time-of-day, using a restricted maximum likelihood methodology. A random effect was included to account for the non-independence between observations from the same participant. For non-parametric data, Wilcoxon signed-rank test was performed. Data are shown as mean±standard deviation (SD), unless otherwise indicated. A p-value less than 0.05 defined statistical significance.

### Results

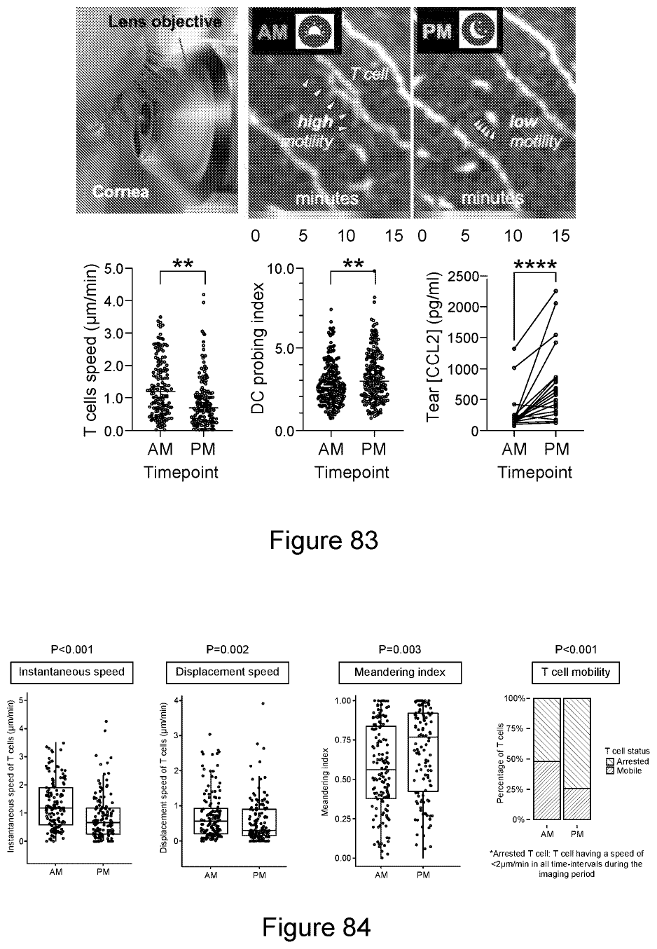

Data are reported for 12 healthy adults, with ages ranging from 20 to 45 years. FIG. 83 summarises the inventor's key findings, whereby while corneal T cell motility was dampened in the evening (PM) relative to the morning (AM), dendritic cells (DCs) showed enhanced morphodynamic activity. This diurnal cell signal was reflected in the tears, with differential levels of the chemoattractant CCL2.

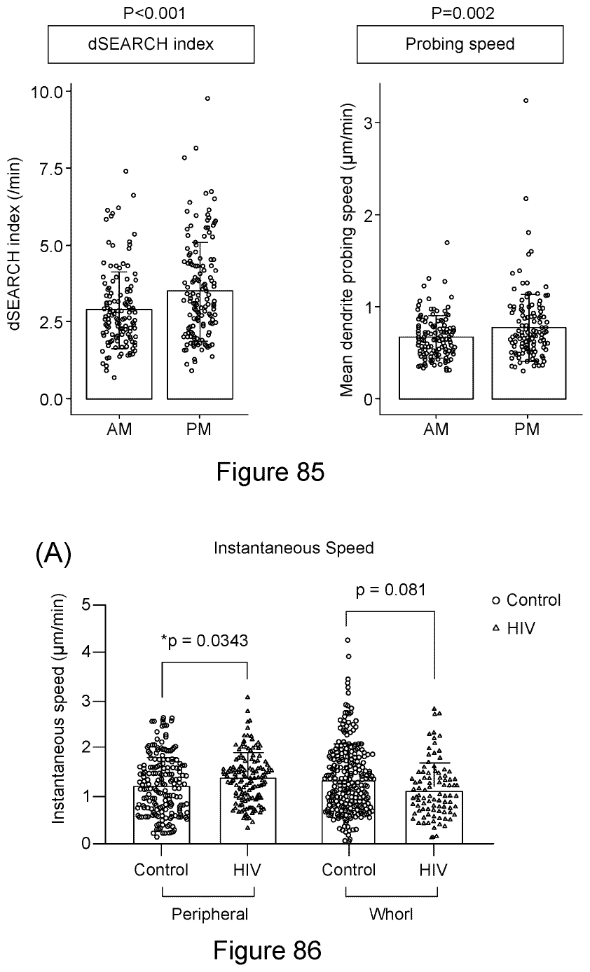

The inventors evaluated both T cells and dendritic cells across the two visits. The inventors found that T cell density, in the same tissue location, was unchanged across time-points (p>0.05). Based on the analysis of 267 corneal T cells in total, FIG. 84 provides further evidence for difference in motility parameters between AM vs PM. Overall, T cells moved less in the afternoon/evening relative to the morning. Dendritic cell (DC) density, in the same tissue location, was unchanged across time-points (p>0.05). Based on the analysis of 103 corneal DCs in total, FIG. 85 provides further evidence for difference in dynamic behaviours between AM vs PM. Overall DCs, had higher dynamic activity in the afternoon/evening relative to the morning.

The inventors have therefore provided the first ever in vivo insight into the temporal regulation of human peripheral tissue immunity. These data provide evidence for the ability to quantify diurnal (temporal) differences in the behaviours of different immune cell subsets using our imaging approach. The inventors have shown that the pattern of change from morning to afternoon/evening is distinct across lymphoid (T cell) and myeloid (DC) derived cells, demonstrating the utility of the time-lapsed imaging approach for quantifying differences in immunobiology across cell classes.

### Example 4—Evidence for the Ability to Use the Imaging Method to Quantify Changes to Corneal Immune Cells in a Systemic Disease (HIV)

### Materials and Methods

Human participants. Ethics approval was obtained from the Alfred Human Research Ethics Committee (ID #191-17). All participants have signed the informed consent before participation. Ocular examinations were conducted in the Department of Optometry and Vision Science at the University of Melbourne, Australia.

This observational, cross-sectional study compared characteristics in healthy (control) participants and individuals positive for human immunodeficiency virus (HIV+). All participants were aged between 60 and 80 years. Eligible participants had normal vision and no clinically significant eye disease, as confirmed by an ocular examination. Eligible participants were required to communicate proficiently in English to complete the surveys for the study. For the HIV+ group, a confirmed diagnosis of HIV was required, as verified through a licensed diagnostic test. All HIV+ participants were managed with antiretroviral therapy (ART) and had not progressed to the clinical stage of acquired immunodeficiency syndrome (AIDS). The main participant exclusion criteria included a diagnosis of diabetes or current prescription of any systemic medications known to affect vision (e.g., antipsychotic medication) or ocular surface health.

Ocular assessments and time-lapsed corneal in vivo confocal microscopy imaging. Participants underwent comprehensive ocular surface examinations, involving collection of information relating to dry eye symptoms, vision level (best-corrected visual acuity), tear film health and corneal health.

Participants' right corneas were imaged using the Heidelberg Retina Tomograph-3 with Rostock Corneal Module, Heidelberg Engineering, Germany, per our protocol.9 The eye to be imaged received one drop of topical anaesthetic prior to imaging. IVCM sequence scans were repeatedly performed at the same corneal region, five-minutes apart, at up to six timepoints. For this study, two distinct areas of cornea were analysed: the corneal ‘whorl’ region (paracentral inferior-nasal cornea) and the ‘peripheral’ cornea (located ~2 mm inferior to the whorl). Time-lapsed videos (25 minutes) were created using a customised software package in MATLAB at both the corneal whorl and peripheral cornea for all participants. Immune cell analyses were performed in Image J. A suite of parameters was used to define the morphology and dynamic behaviours of the immune cell subsets.

Corneal nerve parameters in the central and whorl regions of the cornea were quantified from 8-12 non-overlapping images (400×400 microns) of the central cornea using the automated ACCMetrics software.21 The quantified nerve parameters were: corneal nerve fiber density (CNFD, density of total nerve fibers, fibers/mm²), corneal nerve branch density (CNBD, density of the main nerve fiber branches, branches/mm²), corneal nerve fiber length (CNFL, length of the nerve fibers, mm/mm²), and corneal nerve total branch density (CTBD, density of total nerve fiber branches, total branches/mm²).

Statistical analyses. Corneal immune cell density and nerve parameters were analysed at the “per participant” level, using a t-test or Mann-Whitney test, as appropriate for the data (i.e., normal or non-normally distributed respectively). The immune cell morphological and dynamic parameters were analysed at the “per cell” level, by fitting a linear mixed-effects model using Restricted Maximum Likelihood (for parametric data) or Gauss-Hermite quadrature (for binary data). A random effect was incorporated to accommodate the correlation observed among immune cells originating from the same subject. The statistical analyses were performed using R software (Version 4.3.0, R Development Core Team, https://www.r-project.org/). A p-value less than 0.05 was considered statistically significant.

### Results

This study involved a total of 31 participants, comprising 15 healthy controls (HIV-group; age range: 62 to 79 years) and 16 individuals with HIV infection (HIV+ group; age range: 60 to 79 years). Key demographic and clinical information are summarised in Table 5. There was no difference in clinical ocular assessment between the two groups, including dry eye symptoms (OSDI score), tear osmolarity, tear break-up time and corneal fluorescein staining score. All of the HIV+ patients had been receiving ART treatment for 214±115 months prior to study enrolment, leading to a well-controlled status, including most recent circulating CD4+ T cell counts of 738±359 cells/mm³ and viral load of 10.7±31.1 copies/mL.

TABLE 5
Demographic and clinical characteristics of the study participants

| | Control (HIV−) | HIV+ | |
|---|---|---|---|
| | (n = 15) | (n = 16) | p-value |
| Age (years) | 71.3 ± 5.2 | 67.3 ± 6.2 | 0.060 |
| Sex (M:F) | 14:1 | 15:1 | 0.962 |
| Ocular assessment | | | |
| OSDI score (0-100) | 3.6 ± 2.4 | 3.0 ± 4.3 | 0.635 |
| Tear osmolarity (mOsm/L) | 296.2 ± 9.9 | 302.2 ± 10.2 | 0.108 |
| Tear break-up time (sec) | 7.2 ± 2.3 | 6.9 ± 2.1 | 0.627 |
| Corneal fluorescein staining | 0.9 ± 1.1 | 0.4 ± 0.6 | 0.141 |
| HIV-related metrics | | | |
| Duration since initial diagnosis (months) | — | 214 ± 115 | — |
| CD4+ T cell count (cells/mm³) | — | 738 ± 359 | — |
| HIV viral load (copies/mL) | — | 10.7 ± 31.1 | — |

OSDI: Ocular Surface Disease Index

The inventors found that a higher corneal nerve fibre width was observed in HIV+ individuals relative to HIV− healthy controls (0.022±0.001 vs 0.020±0.001, p=0.004). All other corneal nerve parameters were similar between groups.

Overall, corneal T cell, DC and macrophage densities were similar between participant groups, across each of the central, whorl and peripheral regions of the cornea (p>0.05 for all comparisons). The morphology and dynamic behaviours of DCs and stromal macrophages were also similar across the groups (p>0.05 for all comparisons).

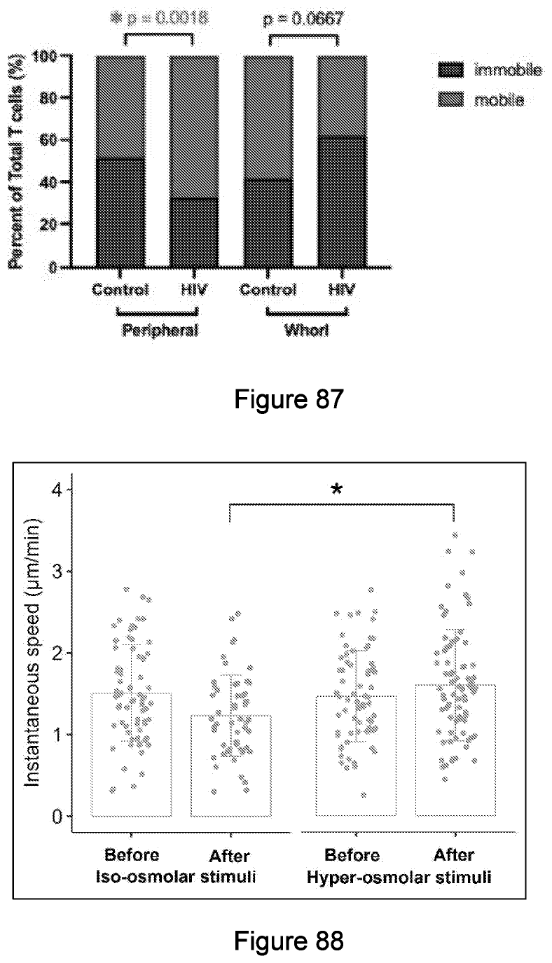

The most striking inter-group difference was that corneal T cells in HIV+ individuals had a higher trajectory (instantaneous) speed than T cells in controls (1.51±0.53 vs 1.21±0.59 μm/min, p=0.0343) in the peripheral region of the cornea (FIG. 86). The higher dynamic activity of T cells in the HIV+ group was also evidenced by a higher percentage of mobile T cells (speed >2 um/min) compared to the control group (FIG. 87; 67% vs 48%, p=0.0018).

In summary, the new IVCM technique revealed that dynamics of corneal intraepithelial T cells are altered in HIV+ individuals with continuing ART treatment. The inventors demonstrated that time-lapsed corneal imaging can be used as a tool to assess corneal immune cell profiles in systemic disease, to identify potential indicators of immune-related diseases and conditions.

### Example 5—Evidence for the Ability to Use the Imaging Method to Quantify Changes to Corneal Immune Cell Dynamics in Response to a Topical Pro-Inflammatory Stimulus (Hyperosmotic Saline)

### Materials and Methods

Human participants. Ethics approval was obtained by the University of Melbourne Human Research Ethics Committee (HREC ID #13783), and received all required institutional clinical trial governance approvals prior to study commencement. Written informed consent to participate was obtained from all participants.

Healthy adult participants in good general health, aged 18 to 45 years were recruited. Key participant inclusion criteria included: not being pregnant, not currently breastfeeding, not having an ocular or systemic disease known to affect the health of small nerve fibres or the ocular surface (e.g., diabetes, Sjogren's syndrome), no prior history of laser refractive eye surgery, not have had active ocular inflammation or infection in the past 3 months, not be currently or recently (within last 3 months) using agents known to affect ocular surface health (e.g., corticosteroids, anti-histamines) or have any corneal conditions that may adversely alter corneal nerves (e.g., post-trauma, contact lens wear, history of herpetic keratitis).

Study assessments. For this study, participants were asked to attend a total of four study visits. All participants underwent screening and baseline clinical assessments at Visit 1. Visit 2 involved a second cross-sectional intervention study. Visits 3 and 4 comprised a randomised, controlled, two-arm, cross-over intervention study (and are the focus of the data reported here).

At Visit 1, following provision of written informed consent, participants underwent comprehensive ocular surface examinations, including the quantification of dry eye symptoms (OSDI), measurement of vision (visual acuity), tear osmolarity, non-invasive tear break-up time, tear meniscus height, basal tear collection, non-contact corneal sensitivity, slit lamp examination of the anterior eye, tear secretion (Schirmer test) and time-lapsed in vivo corneal imaging. For Visits 2, 3 and 4: For all eligible participants, the right eye was the study eye.

At Visit 2 (7±3 days after Visit 1), data collection focused on assessing the subjective (patient sensation) to corneal exposure to sterile, graded saline (electrolyte) solutions. These data are not reported here.

At Visit 3 (30±5 days after Visit 2) and Visit 4 (7±3 days after Visit 3), data collection focused on investigating in vivo corneal immune responses to topical hyperosmolar stimuli. The order of administering either sterile 1500 mOsm/L (4.3%) or 300 mOsm/L (0.9%) saline solutions (involving 10 repeat drops to the right eye, applied 3 minutes apart) were randomised, at the participant level, at Visits 3 and 4.

At Visits 3 and 4, time-lapsed in vivo corneal confocal microscopy imaging was performed at up to six time-points, with a time interval of about 4-8 minutes between each time point, for about 20 minutes both at the start of the visit (before stimulus) and after repeated instillation of either 1500 mOsm/L or 300 mOsm/L saline solutions (after stimulus) time-points. A wash-out period (break) of 45 minutes was given after the initial (before) imaging time-point.

Time-lapsed corneal in vivo confocal microscopy imaging. Participants' right corneas were imaged using the Heidelberg Retina Tomograph-3 with Rostock Corneal Module, Heidelberg Engineering, Germany, per our protocol.⁵⁷ The eye to be imaged received one drop of topical anaesthetic prior to imaging. IVCM sequence scans were repeatedly performed at the same corneal region, five-minutes apart, at up to five timepoints. Time-lapsed videos (20-25 minutes) were created using a customised software package in MATLAB. Imaging data were captured at both the corneal whorl and peripheral inferior cornea (as previously defined). Cell analyses were performed in Image J. A suite of parameters was used to define the morphology and dynamic behaviours of the immune cell subsets.

Statistical analysis. Statistical analyses were performed using R software (Version 4.3.0, R Development Core Team, https://www.r-project.org/). Immune cell density data were analysed at the “per participant” level. Immune cell morphological and dynamic parameters were analyzed at the “per cell” level. A linear mixed-effects model was fitted to test the effect of time-of-day, using a restricted maximum likelihood methodology. A random effect was included to account for the non-independence between observations from the same participant. For non-parametric data, Wilcoxon signed-rank test was performed. Data are shown as mean±standard deviation (SD), unless otherwise indicated. A p-value less than 0.05 defined statistical significance.

### Results

Data are presented from 11 healthy adult participants (2 male, 9 female; aged: mean±SD: 30.6±7.2 years).

In total, 271 corneal epithelial T cells were analysed at the corneal whorl region from the study population (n=11 participants). The inventors found that T cell density was similar to baseline (before) following exposure to both iso- and hyper-osmotic topical stimulus (p>0.05). The instantaneous speed of corneal T cells was significantly higher after exposure to hyper-osmolar saline eye drops, relative to the iso-osmolar condition (1.60±0.68 μm/min vs 1.23±0.50 μm/min; p=0.038, FIG. 88). T cell displacement speed and meandering index were not significantly altered by exposure to either osmotic stimulus (p>0.05 for all comparisons). In addition, the baseline (before stimulus) density and dynamic parameters of T cells were similar between the one-week study visits, indicating the repeatability of these parameters using the corneal immune cell imaging technique.

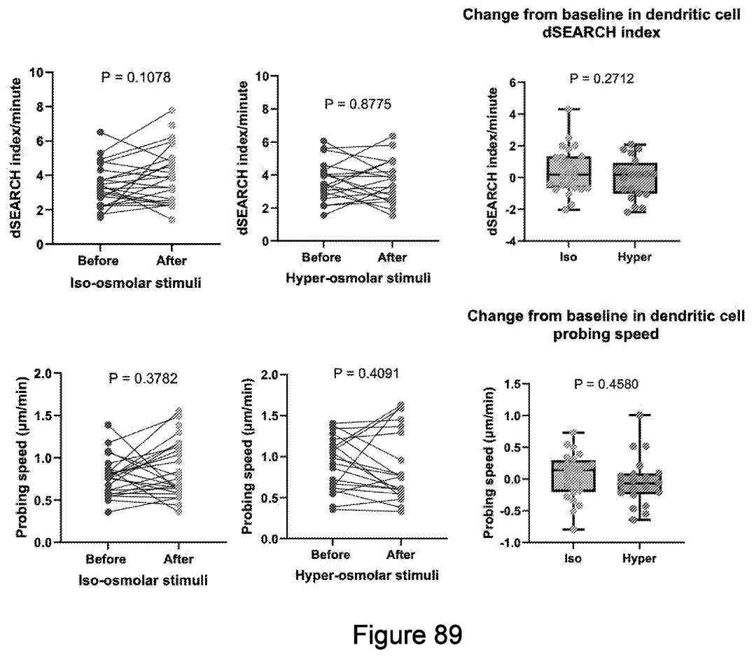

A total of 92 ‘matched’ corneal epithelial DCs (i.e., involving quantification of the same DC both before and after the stimulus) were analysed from the peripheral inferior corneal region, from 10 healthy participants. At the participant level, there were no differences in DC density before and after the exposure to the iso- and hyper-osmotic topical stimulus (p>0.05). At the cell level, dynamic parameters of DCs (i.e., the dendrite surveillance extension and retraction cycling habitude, probing speed) and other morphological parameters (area, field area, perimeter, aspect ratio, circularity, solidity) were also not significantly altered to either osmotic stimulus (FIG. 89).

In summary, the new IVCM imaging technique enabled the in vivo capture and quantification of dynamic changes to human corneal immune cell behaviours in response to topical stimuli. Acute, ocular surface exposure to a pro-inflammatory hyperosmotic stimulus rapidly altered the patrolling characteristics of corneal T cells, indicating the dynamic responsivity of these cells to changes in the local microenvironment.

The inventors therefore demonstrated the capacity for the new IVCM imaging technique to monitor changes in immune cell behaviour in response to a topical (locally applied) pro-inflammatory stimulus (in this case hyperosmotic saline). The inventors have also shown the utility of the technique to delineate a differential dynamic response in one cell class (in this case T cells), in the presence of no apparent change to immune cell dynamics in another cell class (in this case dendritic cells). The inventors have also demonstrated that the technique may be used to quantify the same cell (DC) longitudinally, over an acute time period (a few hours), as thus capture the effect of a stimulus/intervention at an individual cell level, and yield repeatable results over a one-week imaging window (comparing the ‘before’ imaging data across study visits, which are highly consistent).

### Example 6—Evidence for the Ability to Apply the System and Method of Image Processing to the Novel Imaging Technique

Using the system and method 9000 described herein, and applied to corneal IVCM image acquisition and subsequent processing, the inventors acquired data from 68 human research participants, including repeats at 4 to 6 time-points for each individual. At each time-point, a video of 100 unique images or frames was acquired. These time-lapsed ‘sequence mode’ images were acquired at a designated tissue depth (in the z direction) with a consistent tissue depth for all acquisitions, for both the corneal anterior stroma and basal epithelium, for a total of 311 sequence-scan video captures.

TABLE 6
Success of automated corneal IVCM image registration protocol across four clinical studies

| | No. of | | Successful | | Successful | | Analyzable |
|---|---|---|---|---|---|---|---|
| | participants | Corneal | sequence image | | time-lapsed | | tissue area |
| | (No. of | region and | registration | | image video | | (mean ± SD, |
| | eyes) | depth | (montaging) | | creation | | mm²) * |
| Study 1 | 18 (18) | Whorl | 98.96% | (190/192) | 100.00% | (36/36) | 0.202 ± 0.033 |
| (Diurnal | | epithelium | | | | | |
| study) | | Peripheral | 97.65% | (166/170) | 90.63% | (29/32) | 0.175 ± 0.036 |
| | | epithelium | | | | | |
| Study 2 | 20 (20) | Whorl | 94.34% | (100/106) | 95.00% | (19/20) | 0.226 ± 0.037 |
| (HIV | | epithelium | | | | | |
| study) | | Peripheral | 97.50% | (78/80) | 93.75% | (15/16) | 0.224 ± 0.045 |
| | | epithelium | | | | | |
| Study 3 | 13 (13) | Whorl | 96.05% | (73/76) | 92.31% | (12/13) | 0.221 ± 0.030 |
| (Ageing | | epithelium | | | | | |
| study, only | | Peripheral | 98.41% | (62/63) | 90.91% | (10/11) | 0.217 ± 0.031 |
| young ones) | | epithelium | | | | | |
| Study 4 | 17 (17) | Whorl | 98.53% | (334/339) | 94.12% | (64/68) | 0.218 ± 0.055 |
| (LASIK | | epithelium | | | | | |
| study) | | Peripheral | 95.86% | (278/290) | 84.75% | (50/59) | 0.223 ± 0.069 |
| | | epithelium | | | | | |
| | | Whorl stroma | 92.78% | (257/277) | 75.00% | (42/56) | 0.134 ± 0.070 |
| TOTAL | 68 (68) | | 96.55% | (1538/1593) | 89.39% | (278/311) | 0.201 ± 0.062 |

* Overlapped area across all the images or frames in the final successful time-lapsed video for analysing immune cell dynamics (Tissue area imaged in a single IVCM image is 0.16 mm²)

Table 6 summarizes the number of video image sequences acquire an the percentage of successful sequence image registrations (montages), judged by achieving a high-quality averaged image from a single, sequence scan video. Table 6 also includes the number of research participant datasets for which at least four consecutive time points were deemed suitable for registering in a time-lapsed manner, alongside the number of successful time-lapsed videos created. Overall, the protocol was successful in registering 1538 of 1593 (96.55%) of images, and generating 278 of 311 (89.39%) time-lapsed videos.

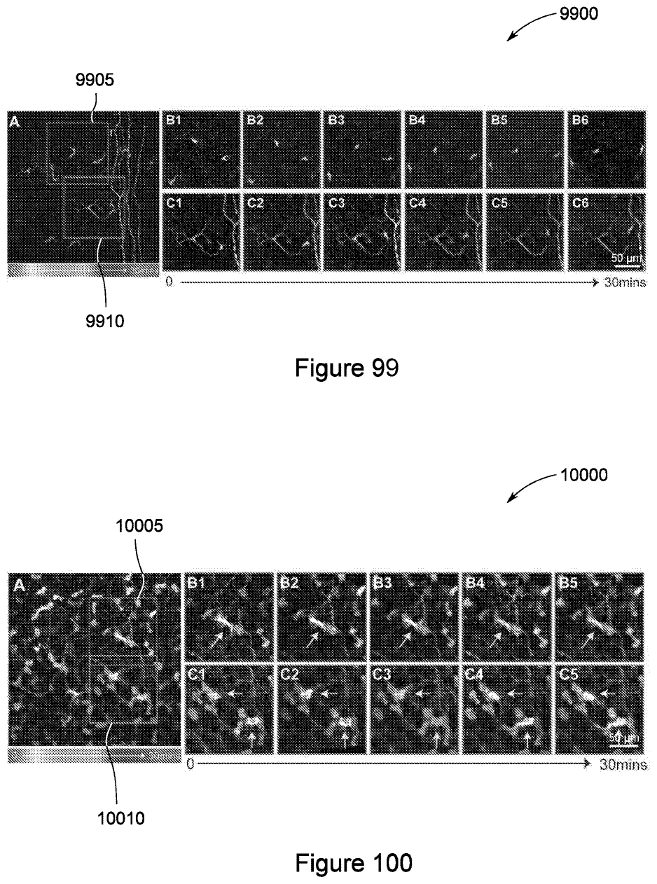

The performance of the system and method are shown in 9900 of FIG. 99 and 10000 of FIG. 100 which illustrate representations of high-quality registered time-lapsed videos showing cellular motility of both putative T cells (red panel 9905, 10005) and dendritic cells (blue panels 9910, 10010) in the human corneal epithelium (FIG. 99), and macrophages in the human corneal stroma (FIG. 100).

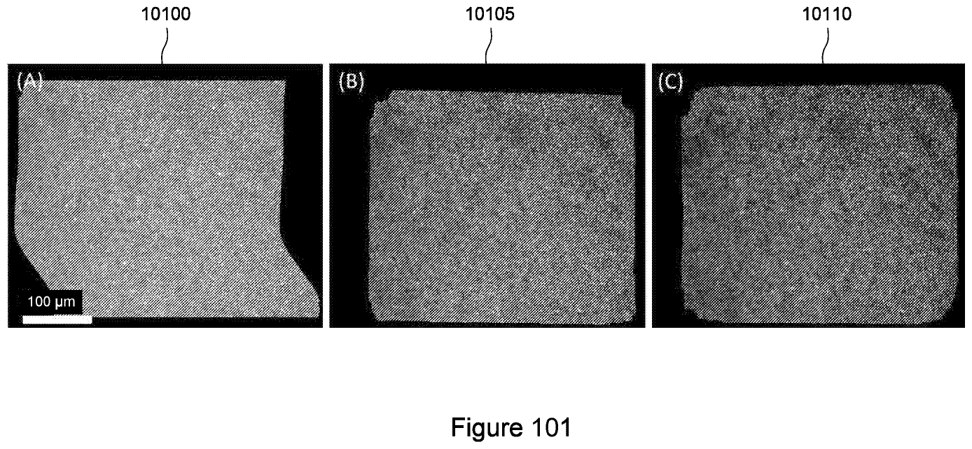

Equally the system and method have utility on a different scanning instrument, where it was applied to a different in vivo confocal microscope—a prototype AOSLO. The scanning instrument was used to acquire 100 image or frame videos at 15 fps, using an imaging wavelength of 790 nm and with fixation directed 0.5° from the foveal center. The same registration protocol described above was used. Of 17 videos acquired during a one-hour imaging session, two were rejected due to poor quality and the remaining 15 were automatically co-registered without further manual input (in particular, without manual selection of a reference image or frame or any other quality control). Examples from the registration process are shown in image 10100, 10105 and 10110 of FIG. 101A, FIG. 101B and FIG. 101C respectively. FIG. 101A shows a single registered image or frame 10100 whose borders appear highly warped due to successful tracking of a large microsaccade. FIG. 101B shows the strip-registered average 10105 of the corresponding 100 image/frame video sequence. The sequence contains several microsaccades, and intensity is seen to decline over time due to error in the adaptive optics correction; these sources of registration noise appeared to be handled well with no overt errors. Data from all images or frames contributed to the averaged image, i.e. “lucky averaging” was not applied in this case. FIG. 101C shows the average 10110 of 15 such averaged images collected over a one-hour period. The visibility of cones to within ~0.2° of the foveal center (bottom left), and the beginning of rod intrusion within the cone mosaic (top right) confirm the fidelity of the registration method, and demonstrate the broad applicability of this registration protocol.

### Summary of Examples—a New Model of Steady-State Human Corneal Immunology

Collectively, the data from Examples 1-5 show the new IVCM technique to be a versatile, non-invasive tool for studying the in vivo distribution, morphology and dynamic behaviours of immune cell populations in the human cornea. Insights provided by time-lapsed corneal in vivo confocal microscopy imaging, married with the inventors' complementary in vivo and ex vivo analyses of immune cells in mice, and ex vivo analyses of donated healthy corneas, redefine present understanding of human corneal immunology.

The currently accepted model of the principle immune cell subsets in the healthy human cornea, involving “mature” and “immature” DCs in the epithelium (as imaged using traditional static IVCM) and macrophages in the stroma. Based on the time-lapsed corneal in vivo confocal microscopy imaging data, the inventors posit a role for adaptive immune cells (T cells) in homeostatic human corneal immune surveillance. Using this new IVCM imaging technique, the inventors identified motile T cells that patrolled the epithelium, and DCs that show cyclic dSEARCH behaviours; these immune cell subsets interact with each other (cell-to-cell interaction) and with sensory nerves (neuro-immune interaction). In the corneal stroma, macrophages are observed to crawl between keratocytes, and to use membrane extensions consistent with the lamellipodia and/or filopodia of macrophages in other tissues.

The new IVCM imaging technique enables insight into the in vivo effects of chronic and acute inflammation on the distribution, morphology and dynamic behaviours of corneal immune cell subsets. In humans with symptomatic untreated seasonal allergy, T cell motility is enhanced, DC numbers are reduced, and the DCs that are present show reduced tissue surveillance. Macrophages are reduced in density and show reduced cell areas. In response to an acute pro-inflammatory ocular surface stimulus (CL wear), epithelial T cells enhance their motility and are reduced in density in the paracentral cornea; DCs show reduced dSEARCH. Stromal macrophages change their morphological conformation, with an increased number of cellular protrusions, larger cell area and reduction in circularity.

In contrast to the long-held view that the healthy human cornea only houses innate immune cells, the inventors have shown that innate and adaptive immune cells co-exist in the healthy human cornea, including a putative population of T_{RM} cells that patrol the epithelium under homeostatic conditions. Moreover, the inventors have demonstrated that chronic and acute ocular surface inflammatory stimuli induce differential effects on innate (i.e., DC and macrophage) and adaptive (T cell) immune cell subtypes, spanning the epithelial and stromal corneal layers.

### Advantages of the Disclosure

Embodiments of the present disclosure provide new methods for IVCM and demonstrate the capacity for in vivo, label-free longitudinal evaluation of immune cell shape (morphological tracking) and movement (temporal/spatial tracking) through human corneal tissue (depth tracking). The method can be used to image the corneal immune compartment and capture comprehensive insights into the dynamic behaviours of immune cell subsets in healthy human eyes and in response to inflammation. The differential effects of chronic and acute inflammation on the distribution and behaviour of immune cells have been shown and the restorative effects of immunomodulatory therapeutics on adaptive immune cell dynamics have been demonstrated in subjects treated for seasonal allergy.

The IVCM data from Examples 1-5 has identified three distinct immune cell subsets in the healthy human cornea, distinguished by cell morphology and dynamics. This includes: (i) T cells: small, motile cells whose shape and movement parallel T cells seen using intravital 2-photon imaging in mice after viral infection¹⁹, (ii) DCs: larger cells with obvious dendrites populating the epithelium that show minimal lateral migration^{18, 28}; and (iii) macrophages: amoeboid and/or star-shaped cells in the stroma that appear to crawl between keratocytes²⁹. The inventors have shown that the morphological and dynamic features of these corneal immune cell subsets, imaged in living humans in vivo, are congruent with those observed in mice, analysed using in vivo and ex vivo approaches. Flow cytometry on healthy human donor corneas provide evidence for the presence of CD3⁺ T cells in the epithelium and stroma, and smaller percentages of DCs and macrophages. While flow cytometry data was supportive of the time-lapsed corneal in vivo confocal microscopy dynamic cell phenotyping, in that T cells were observed in all samples, this comparison should be interpreted in the context of the relatively confined area of tissue captured using time-lapsed corneal in vivo confocal microscopy imaging (i.e., the central and mid-peripheral cornea), compared to flow cytometry that involves analysis of entire corneal samples (i.e., both the central and peripheral regions).

The cornea is considered an ‘immune privileged’ site, due to its avascularity, tight surface barriers and regulated immune cell surveillance³⁰. Current understanding of its immune cell landscape derives primarily from mouse studies, with presumed generalizability to humans. The steady-state cornea is generally accepted to house DCs in the epithelium and macrophages in the stroma^{31, 32, 33, 34}. Over 200 publications over the past 20 years that have evaluated human corneal immune cells using static IVCM refer to immune cells in the epithelium as DCs or Langerhans cells. The present data challenges this dogma^{35, 36}, revealing an unrecognised population of patrolling T cells. The inventors recently reported that HSV-1 infection induces the formation of tissue-resident memory T (T_{RM}) cells in the mouse cornea; these cells closely match the morphology and dynamic behaviours of epithelial immune cells in normal human corneas¹⁹. However, none of the 30 adult participants in the current study had a known history of corneal infection, and putative T cells were imaged in all individuals. Common and less severe ocular surface infection (e.g., viral conjunctivitis) or exposure to environmental antigens may be sufficient to induce corneal T_{RM} formation, and T_{RM} phenotype cells exist in the human conjunctiva³¹.

Using the new IVCM technique, the inventors observed dynamic interactions between corneal immune cells. The inventors described DC-T cell interactions and T cell-T cell interactions, lasting several minutes. The inventors also uniquely captured in vivo sensory nerve-immune cell interactions. Neuro-immune crosstalk is established in barrier surfaces such as the gut, skin and airway³⁸, with growing evidence for similar interactions in the cornea^{35, 39, 40}. Limitations of many human studies to date include use of static image analyses that poorly capture spatio-temporal dynamics, and challenges with deeper-tissue imaging³⁸. Time-lapsed corneal in vivo confocal microscopy imaging overcomes these pitfalls. In the healthy cornea, DC-nerve interactions were frequent and dynamic, with dendritic tips episodically interacting with nerve axons. These findings align with data showing the structural interdependence of epithelial DCs and sensory nerves in the mouse cornea⁴¹. which has been linked to nerve function and repair^{41, 42}. The inventors also noted ‘kissing’ interactions between presumed T cells and nerves that appeared to lead to increased cell motility post-contact. Capacity to capture these fundamental interactions between the nervous and immune systems in the living human eye using the new IVCM technique provides opportunities to study the interface between nerves and immune cells in an intact peripheral sensory tissue, including how exogenous factors and disease may disrupt the perception, integration and responsiveness of cell subtypes to different challenges.

The new IVCM technique was shown to define changes to corneal immune cells under non-homeostatic conditions, by evaluating eyes with symptomatic seasonal allergy (as a model of chronic immune activation)⁶, and before and after CL exposure (as an acute inflammatory stimulus⁴⁰). The inventors discovered differential effects on the density, morphology and behavior of immune cell subsets. Corneal T cells in people with symptomatic eye allergy had higher motility and a lower arrest coefficient, indicating heightened tissue surveillance⁴³. Although the mechanism through which T cells adapt their motility is unclear⁴⁴, tissue inflammation can increase T cell speed in vivo⁴⁵. The increased adaptive corneal immune cell response in ocular allergy was accompanied by a relative suppression of innate immune cells, with fewer epithelial DCs, reduced dSEARCH behaviours, and lower stromal macrophage density. As subsets of DCs regulate the induction of various T cell subtypes that suppress allergic responses⁴⁶, our findings raise the possibility of a relative reduction in homeostatic anti-inflammatory corneal tissue responses in individuals experiencing ocular allergy. The new IVCM technique also showed a relative normalization of T cell dynamics in individuals treated with corticosteroids or allergen-specific immunotherapy for seasonal allergy. Glucocorticoids modulate peripheral immune responses by inhibiting T cell immunity⁴⁷, while immunotherapy restores a state of natural tolerance to an allergen that is similar to non-allergic individuals⁴⁸. The quantified stabilization of T cell behaviour was associated with low ocular symptoms, highlighting the potential utility of time-lapsed corneal in vivo confocal microscopy imaging to identify immune cell dynamics as biomarkers of therapeutic response.

Modelling acute ocular surface inflammation, three hours of CL wear led to reduced dSEARCH activity in individual epithelial DCs (i.e., the same cells imaged pre- and post-CL wear). T cell numbers were halved in the paracentral corneal epithelium, with remaining cells showing an ~30% increase in motility. The rapid decline in cell density may reflect either migration towards the corneal periphery and/or cell movement from the epithelium to the stroma. The potential for immune cells to traffic through the epithelial basement membrane via nerve branch points has been described for “round shaped DCs” in the mouse cornea⁴¹. In addition, in the underlying stroma macrophages showed plasticity in cell shape, characterized by the appearance of additional cellular protrusions. Changes to cell shape can reflect altered cell function⁴⁹, mediated by interactions between the extracellular matrix and cell surface adhesion proteins, and changes to intracellular contractility⁵⁰. Consistent with their essential role in innate immunity, including host defence and wound healing, macrophage behaviour is primarily regulated by soluble factors in their microenvironment⁵¹. The new IVCM technique reveals that exposing the ocular surface to a CL provokes a robust local tissue immune response that is not limited to the (outer) epithelium, but also affects the stroma, suggesting active communication mechanisms across these tissue layers. The differential responses of stromal macrophages in acute (i.e., CL wear) and chronic (i.e., allergy) inflammatory states was interesting, with macrophages appearing smaller in corneas of allergy participants, but showing an increase in cell size and lower circularity following a few hours of CL wear. Macrophage morphology (i.e., cell area and perimeter) can be an indicator of cell activation state and phenotype. Recent immunohistochemical evidence from tumour biopsies suggest that larger tumour-associated macrophages in colorectal liver metastasis patients are prognostic of lower five-year survival⁵². How the observed differences in macrophage morphology in acute versus chronic inflammatory scenarios in this present study relate to macrophage activation is unclear, but support a role for the new IVCM technique as a means to define the in vivo inflammatory status of the human cornea.

To the inventors' knowledge, these data represent the first demonstration of the dynamic behaviours of immune cell subsets in the human cornea, and inducible tissue-resident immune cell activation in response to a stimulus. Together, the data establishes the utility of the new IVCM technique as a unique tool to identify concurrent alterations to the static and dynamic features of T cells, DCs and macrophages, across the depth of the tissue. The skilled person would therefore appreciate that the present invention has the capacity to be used to detect changes in cell dynamics in other regions of the eye (for example, the retina).
[1] Stachs O., G. R. F., Aumann S. In Vivo Confocal Scanning Laser Microscopy, vol. Chapter 12. Springer: Cham (CH), 2019.
[2] Jester, J. V., Andrews, P. M., Petroll, W. M., Lemp, M. A. & Cavanagh, H. D. In vivo, real-time confocal imaging. Journal of electron microscopy technique 18, 50-60 (1991).
[3] Mobeen, R. et al. Corneal epithelial dendritic cell density in the healthy human cornea: A meta-analysis of in-vivo confocal microscopy data. Ocul Surf 17, 753-762 (2019).
[4] Liu, J. & Li, Z. Resident Innate Immune Cells in the Cornea. Frontiers in immunology 12, 620284 (2021).
[5] Lagali, N. S. et al. Dendritic cell maturation in the corneal epithelium with onset of type 2 diabetes is associated with tumor necrosis factor receptor superfamily member 9. Scientific reports 8, 14248 (2018).
[6] Galli, S. J., Tsai, M. & Piliponsky, A. M. The development of allergic inflammation. Nature 454, 445-454 (2008).
[7] Efron, N. Contact lens wear is intrinsically inflammatory. Clinical and Experimental Optometry 100, 3-19 (2017).
[8] The University of Melbourne. Why is grass pollen only counted between 1 October and 31 December? [Accessed 27 Jun. 2022] Available from: https://www.melbournepollen.com.au/faqs/why-grass-pollen-only-counted-between-1-october-and-31-december/[9]
[9] Alexander, M. et al. The reliability, validity, and preliminary responsiveness of the Eye Allergy Patient Impact Questionnaire (EAPIQ). Health and quality of life outcomes 3, 67 (2005).
[10] Wolffsohn, J. S. et al. TFOS DEWS II Diagnostic Methodology report. Ocul Surf 15, 539-574 (2017).
[11] Zhang, X. Y., Wu, M., Chinnery, H. R. & Downie, L. E. Defining an Optimal Sample Size for Corneal Epithelial Immune Cell Analysis Using in vivo Confocal Microscopy Images. Frontiers in medicine 9, 848776 (2022).
[12] De Silva, M. E. H., Zhang, A. C., Karahalios, A., Chinnery, H. R. & Downie, L. E. Laser scanning in vivo confocal microscopy (IVCM) for evaluating human corneal sub-basal nerve plexus parameters: protocol for a systematic review. BMJ open 7, e018646 (2017).
[13] Britten-Jones, A. C. et al. Investigating the Neuroprotective Effect of Oral Omega-3 Fatty Acid Supplementation in Type 1 Diabetes (nPROOFS1): A Randomized Placebo-Controlled Trial. Diabetes 70, 1794-1806 (2021).
[14] Colorado, L. H., Edwards, K., Chinnery, H. R. & Bazan, H. E. In vivo immune cell dynamics in the human cornea. Experimental eye research 199, 108168 (2020).
[15] Dehghani, C. et al. Morphometric Changes to Corneal Dendritic Cells in Individuals With Mild Cognitive Impairment. Frontiers in neuroscience 14, 556137 (2020).
[16] Kheirkhah, A. et al. Corneal Epithelial Immune Dendritic Cell Alterations in Subtypes of Dry Eye Disease: A Pilot In Vivo Confocal Microscopic Study. Investigative ophthalmology & visual science 56, 7179-7185 (2015).
[17] Senthil, K., Jiao, H., Downie, L. E. & Chinnery, H. R. Altered Corneal Epithelial Dendritic Cell Morphology and Phenotype Following Acute Exposure to Hyperosmolar Saline. Investigative ophthalmology & visual science 62, 38 (2021).
[18] Nishibu, A. et al. Behavioral responses of epidermal Langerhans cells in situ to local pathological stimuli. The Journal of investigative dermatology 126, 787-796 (2006).
[19] Loi K. J., A. Y. O., Senthil K., Schienstock D., Sandford S., Devi S., Christo S. N., Mackay L. K., Chinnery H. R., Osborne P. B., Downie L. E., Sloan E. K., Mueller S. N. Corneal tissue resident memory T cells form a unique immune compartment at the ocular surface. Cell Reports 39, 110852 (2022).
[20] Mueller, S. N., Heath, W., Mclain, J. D., Carbone, F. R. & Jones, C. M. Characterization of two TCR transgenic mouse lines specific for herpes simplex virus. Immunology and cell biology 80, 156-163 (2002).
[21] Bertram, K. M. et al. Identification of HIV transmitting CD11c (+) human epidermal dendritic cells. Nature communications 10, 2759 (2019).
[22] Haniffa, M. et al. Differential rates of replacement of human dermal dendritic cells and macrophages during hematopoietic stem cell transplantation. The Journal of experimental medicine 206, 371-385 (2009).
[23] McGovern, N. et al. Human dermal CD14+ cells are a transient population of monocyte-derived macrophages. Immunity 41, 465-477 (2014).
[24] Rhodes, J. W. et al. Human anogenital monocyte-derived dendritic cells and langerin+cDC2 are major HIV target cells. Nature communications 12, 2147 (2021).
[25] Chinnery, H. R. et al. The chemokine receptor CX3CR1 mediates homing of MHC class II-positive cells to the normal mouse corneal epithelium. Investigative ophthalmology & visual science 48, 1568-1574 (2007).
[26] Alzahrani, Y., Pritchard, N. & Efron, N. Changes in corneal Langerhans cell density during the first few hours of contact lens wear. Contact lens & anterior eye 39, 307-310 (2016).
[27] Downie, L. E. et al. Modulating Contact Lens Discomfort With Anti-Inflammatory Approaches: A Randomized Controlled Trial. Investigative ophthalmology & visual science 59, 3755-3766 (2018).
[28] Ward, B. R. et al. Local thermal injury elicits immediate dynamic behavioural responses by corneal Langerhans cells. Immunology 120, 556-572 (2007).
[29] Stow, J. L. & Condon, N. D. The cell surface environment for pathogen recognition and entry. Clinical & Translational Immunology 5, e71 (2016).
[30] Taylor, A. W. Ocular Immune Privilege and Transplantation. Frontiers in immunology 7, 37 (2016).
[31] Brissette-Storkus, C. S., Reynolds, S. M., Lepisto, A. J. & Hendricks, R. L. Identification of a novel macrophage population in the normal mouse corneal stroma. Investigative ophthalmology & visual science 43, 2264-2271 (2002).
[32] Hamrah, P., Zhang, Q., Liu, Y. & Dana, M. R. Novel characterization of MHC class II-negative population of resident corneal Langerhans cell-type dendritic cells. Investigative ophthalmology & visual science 43, 639-646 (2002).
[33] Hamrah, P., Huq, S. O., Liu, Y., Zhang, Q. & Dana, M. R. Corneal immunity is mediated by heterogeneous population of antigen-presenting cells. Journal of leukocyte biology 74, 172-178 (2003).
[34] Hamrah, P., Liu, Y., Zhang, Q. & Dana, M. R. The corneal stroma is endowed with a significant number of resident dendritic cells. Investigative ophthalmology & visual science 44, 581-589 (2003).
[35] Frutos-Rincón, L., Gómez-Sánchez, J. A., Íñigo-Portugués, A., Acosta, M. C. & Gallar, J. An Experimental Model of Neuro-Immune Interactions in the Eye: Corneal Sensory Nerves and Resident Dendritic Cells. International journal of molecular sciences 23 (2022).
[36] Niederer, R. L. & McGhee, C. N. Clinical in vivo confocal microscopy of the human cornea in health and disease. Progress in retinal and eye research 29, 30-58 (2010).
[37] Bose, T., Lee, R., Hou, A., Tong, L. & Chandy, K. G. Tissue resident memory T cells in the human conjunctiva and immune signatures in human dry eye disease. Scientific reports 7, 45312 (2017).
[38] Veiga-Fernandes, H. & Mucida, D. Neuro-Immune Interactions at Barrier Surfaces. Cell 165, 801-811 (2016).
[39] Jiao, H. et al. Novel alterations in corneal neuroimmune phenotypes in mice with central nervous system tauopathy. Journal of neuroinflammation 17, 136 (2020).
[40] Wu, M., Hill, L. J., Downie, L. E. & Chinnery, H. R. Neuroimmune crosstalk in the cornea: The role of immune cells in corneal nerve maintenance during homeostasis and inflammation. Progress in retinal and eye research, 101105 (2022).
[41] Gao, N., Lee, P. & Yu, F. S. Intraepithelial dendritic cells and sensory nerves are structurally associated and functional interdependent in the cornea. Scientific reports 6, 36414 (2016).
[42] Gao, N., Yan, C., Lee, P., Sun, H. & Yu, F. S. Dendritic cell dysfunction and diabetic sensory neuropathy in the cornea. The Journal of clinical investigation 126, 1998-2011 (2016).
[43] Krummel, M. F., Bartumeus, F. & Gérard, A. T cell migration, search strategies and mechanisms. Nature Reviews Immunology 16, 193-201 (2016).
[44] Sundqvist, K. G. T Cell Motility—How Is It Regulated? Frontiers in immunology 11, 588642 (2020).
[45] Teijeira, A. et al. T Cell Migration from Inflamed Skin to Draining Lymph Nodes Requires Intralymphatic Crawling Supported by ICAM-1/LFA-1 Interactions. Cell Reports 18, 857-865 (2017).
[46] Akbari, O. & Umetsu, D. T. Role of regulatory dendritic cells in allergy and asthma. Current allergy and asthma reports 5, 56-61 (2005).
[47] Van Laethem, F. et al. Glucocorticoids attenuate T cell receptor signaling. The Journal of experimental medicine 193, 803-814 (2001).
[48] Wambre, E. Effect of allergen-specific immunotherapy on CD4+ T cells. Current opinion in allergy and clinical immunology 15, 581-587 (2015).
[49] Folkman, J. & Moscona, A. Role of cell shape in growth control. Nature 273, 345-349 (1978).
[50] Tan, J. L. et al. Cells lying on a bed of microneedles: an approach to isolate mechanical force. Proceedings of the National Academy of Sciences of the United States of America 100, 1484-1489 (2003).
[51] Mcwhorter, F. Y., Wang, T., Nguyen, P., Chung, T. & Liu, W. F. Modulation of macrophage phenotype by cell shape. Proceedings of the National Academy of Sciences of the United States of America 110, 17253-17258 (2013).
[52] Donadon, M. et al. Macrophage morphology correlates with single-cell diversity and prognosis in colorectal liver metastasis. The Journal of experimental medicine 217 (2020).
[53] Silver, J. D., Spriggs, K., Haberle, S., Katelaris, C. H., Newbigin, E. J., Lampugnani, E. R. Crowd-sourced allergic rhinitis symptom data: The influence of environmental and demographic factors. Sci Total Environ 705, 135147 (2020).
[54] Ong, E. K., Singh, M. B., Knox, R. B. Grass pollen in the atmosphere of Melbourne: Seasonal distribution over nine years. Grana 34 (1), 58-63 (1995).
[55] Bron, A. J., Evans, V. E., Smith J. A. Grading of corneal and conjunctival staining in the context of other dry eye tests. Cornea 22 (7), 640-50 (2003).
[56] Korb, D. R., Herman, J. P., Greiner, J. V., et al. Lid wiper epitheliopathy and dry eye symptoms. Eye Contact Lens 31 (1), 2-8 (2005).
[57] Downie, L. E., Zhang, X., Wu, M., et al. Redefining the human corneal immune compartment using dynamic intravital imaging. Proc Natl Acad Sci USA. August 120 (31), e2217795120 (2023).
[58] Zhang, X. Y., Wu, M., Chinnery, H. R., Downie, L. E. Defining an Optimal Sample Size for Corneal Epithelial Immune Cell Analysis Using in vivo Confocal Microscopy Images. Front Med (Lausanne) 9, 848776 (2022).
[59] McArdle, S., Buscher, K., Ghosheh, Y., et al. Migratory and Dancing Macrophage Subsets in Atherosclerotic Lesions. Circ Res. 125 (12), 1038-1051 (2019).

1-96. (canceled)

97. A method of identifying a condition or disease state in a subject, the method comprising the steps of:
a) determining the morphology, or number, or density, or dynamic behaviour of one or more immune cells at one or more locations within a focal plane (x, y) and/or at one or more focal depths (z) in corneal tissue of the subject over time; and
b) comparing the morphology, or number, or density, or dynamic behaviour of the one or more immune cells at the one or more locations within the focal plane (x, y) and/or at the one or more focal depths (z) in the corneal tissue of the subject over time to a reference level;
wherein a change in one or more of the morphology, or number, or density, or dynamic behaviour of the one or more immune cells as compared to the reference level is indicative of a condition or disease state in the subject.

98. The method according to claim 97, wherein the condition or disease state is chronic inflammation in the eye.

99. The method according to claim 98, wherein the method comprises determining the dynamic behaviour of one or more putative T cells in the epithelium of the corneal tissue, wherein a higher motility of the putative T cells at about the corneal whorl region compared to the reference level is indicative of chronic inflammation in the eye.

100. (canceled)

101. The method according to claim 97, wherein the method comprises determining the density of one or more putative dendritic cells in the epithelium of the corneal tissue, wherein a lower density of the putative dendritic cells at about the corneal whorl region compared to the reference level is indicative of chronic inflammation in the eye.

102. The method according to claim 97, wherein the method comprises determining the dynamic behaviour of one or more putative dendritic cells in the epithelium of the corneal tissue, wherein a lower dynamic activity of the putative dendritic cells at about the corneal whorl region compared to the reference level is indicative of chronic inflammation in the eye.

103. (canceled)

104. The method according to claim 97, wherein the method comprises determining the density of one or more putative macrophage cells in the stroma of the corneal tissue, wherein a lower density of the putative macrophage cells at about the corneal whorl region compared to the reference level is indicative of chronic inflammation in the eye.

105. The method according to claim 97, wherein the method comprises determining the morphology of one or more putative macrophage cells in the stroma of the corneal tissue, wherein putative macrophage cells at about the corneal whorl region having one or more of the following parameters: a deviation in cell area or cell perimeter compared to the reference level, are indicative of chronic inflammation in the eye.

106. (canceled)

107. The method according to claim 97, wherein the reference level is the morphology, number, or average density, or dynamic behaviour of a corresponding cell type at the one or more locations within the focal plane (x, y) and/or at the one or more focal depths (z) in the corneal tissue of a healthy subject or a subject treated or exposed to an agent or a baseline level of the subject.

108. The method according to claim 97, wherein the condition or disease state is acute inflammation in the eye caused by contact lens wear.

109-118. (canceled)

119. A method of treating a condition or disease state in a subject, the method comprising the steps of:
a) determining the morphology, or number, or density, or dynamic behaviour of one or more immune cells at one or more locations within a focal plane (x, y) and/or at one or more focal depths (z) in corneal tissue of the subject over time;
b) comparing the morphology, or number, or density, or dynamic behaviour of the one or more immune cells at the one or more locations within the focal plane (x, y) and/or at the one or more focal depths (z) in the corneal tissue of the subject over time to a reference level; wherein a change in one or more of the morphology, or number, or density, or dynamic behaviour of the one or more immune cells as compared to the reference level is indicative of a condition or disease state in the subject; and
c) selecting a treatment or modifying a treatment, based on the identification of a condition or disease state in a subject.

120. A method of monitoring treatment efficacy of a condition or disease state in a subject, the method comprising the steps of:
a) determining the morphology, or number, or density, or dynamic behaviour of one or more immune cells at one or more locations within the focal plane (x, y) and/or at one or more focal depths (z) in corneal tissue of the subject over time;
b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a responder or non-responder to treatment or to a baseline level before initiation of treatment; and
c) optionally, continuing treatment when the subject is determined to be a responder; or switching the treatment when the subject is determined to be a non-responder, wherein switching the treatment comprises increasing a dose of the treatment administered, adding one or more of agents to the treatment regimen, discontinuing the treatment and initiating a different treatment regimen.

121. A method of diagnosing a condition or disease state in a subject, or predicting the likelihood of a subject developing a condition or disease state, the method comprising the steps of:
a) determining the morphology, or number, or density, or dynamic behaviour of one or more immune cells at one or more locations within the focal plane (x, y) and/or at one or more focal depths (z) in corneal tissue of the subject over time;
b) correlating the morphology, or number, or density, or dynamic behaviour of the one or more immune cells at the one or more locations within the focal plane (x, y) and/or at the one or more focal depths (z) in the corneal tissue of the subject over time to a reference level indicative of a subject with the condition or disease state, or a reference level indicative of a subject likely to develop the condition or disease state; and
c) optionally, administering a treatment or preventative therapy to the subject when the subject is diagnosed with the condition or disease state, or deemed likely to develop the condition or disease state.

122-125. (canceled)

126. A system for identifying a condition or disease state in a subject, the system including: a communication interface; a processor; and a computer-readable medium communicatively coupled to the processor and storing computer-executable instructions that, when executed by the processor, cause the processor to perform the method of claim 97
a).

127. A system for selecting a treatment for a condition or disease state in a subject, the system including: a communication interface; a processor; and a computer-readable medium communicatively coupled to the processor and storing computer-executable instructions that, when executed by the processor, cause the processor to perform the method of claim 119.

128. A system for monitoring treatment efficacy of a condition or disease state in a subject, the system including: a communication interface; a processor; and a computer-readable medium communicatively coupled to the processor and storing computer-executable instructions that, when executed by the processor, cause the processor to perform the method of claim 120.

129. A system for diagnosing a condition or disease state in a subject, or predicting the likelihood of a subject developing a condition or disease state, the system including: a communication interface; a processor; and a computer-readable medium communicatively coupled to the processor and storing computer-executable instructions that, when executed by the processor, cause the processor to perform the method of claim 121.

130-133. (canceled)

134. A non-transitory computer-readable medium for identifying a condition or disease state in a subject, including instructions stored thereon, that when executed on a processor, perform the method of claim 97.

135. A non-transitory computer-readable medium for selecting a treatment for a condition or disease state in a subject, including instructions stored thereon, that when executed on a processor, perform the method of claim 119.

136. A non-transitory computer-readable medium for monitoring treatment efficacy of a condition or disease state in a subject, including instructions stored thereon, that when executed on a processor, perform the method of claim 120.

137. A non-transitory computer-readable medium for diagnosing a condition or disease state in a subject, or predicting the likelihood of a subject developing a condition or disease state, including instructions stored thereon, that when executed on a processor, perform the method of claim 121.

138-144. (canceled)
